SEQUENCE LISTING (108 sequences)

SEQ ID NO: 1 - protein, 320 aa, Artificial Sequence
  Synthetic Construct
MPSEPPFGRHLIFASLTCLIDAVCKKRYHNQNVYILSILRMTRSKPVNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 2 - DNA, 963 bp, Artificial Sequence
  Synthetic Construct
atgccgtctgaaccgccgttcggacgacatttgatttttgcttctttgacctgcctcattgatgcggtatgcaaaaaaagataccataaccaaaatgtttatatattatctattctgcgtatgactaggagtaaacctgtgaatcgaactgccttctgctgcctttctctgaccactgccctgattctgaccgcctgcagcagcggagggggtggtgtcgccgccgacatcggtgcggggcttgccgatgcactaaccgcaccgctcgaccataaagacaaaggtttgcagtctttgacgctggatcagtccgtcaggaaaaacgagaaactgaagctggcggcacaaggtgcggaaaaaacttatggaaacggtgacagcctcaatacgggcaaattgaagaacgacaaggtcagccgtttcgactttatccgccaaatcgaagtggacgggcagctcattaccttggagagtggagagttccaagtatacaaacaaagccattccgccttaaccgcctttcagaccgagcaaatacaagattcggagcattccgggaagatggttgcgaaacgccagttcagaatcggcgacatagcgggcgaacatacatcttttgacaagcttcccgaaggcggcagggcgacatatcgcgggacggcgttcggttcagacgatgccggcggaaaactgacctacaccatagatttcgccgccaagcagggaaacggcaaaatcgaacatttgaaatcgccagaactcaatgtcgacctggccgccgccgatatcaagccggatggaaaacgccatgccgtcatcagcggttccgtcctttacaaccaagccgagaaaggcagttactccctcggtatctttggcggaaaagcccaggaagttgccggcagcgcggaagtgaaaaccgtaaacggcatacgccatatcggccttgccgccaagcaataa

SEQ ID NO: 3 - protein, 280 aa, Artificial Sequence
  Synthetic Construct
MTRSKPVNRTAFCCLSLTAALILTACSSGGGGVAADIGAVLADALTAPLDHKDKSLQSLTLDQSVRKNEKLKLAAQGAEKTYGNGDSLNTGKLKNDKVSRFDFIRQIEVDGQLITLESGEFQVYKQSHSALTALQTEQVQDSEHSGKMVAKRQFRIGDIAGEHTSFDKLPEGGRATYRGTAFGSDDASGKLTYTIDFAAKQGHGKIEHLKSPELNVDLAASDIKPDKKRHAVISGSVLYNQAEKGSYSLGIFGGQAQEVAGSAEVETANGIRHIGLAAKQ

SEQ ID NO: 4 - DNA, 843 bp, Artificial Sequence
  Synthetic Construct
atgactaggagcaaacctgtgaaccgaactgccttctgctgcctttctttgaccgccgccctgattctgaccgcctgcagcagcggaggcggcggtgtcgccgccgacatcggcgcggtgcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaagtgtacaaacaaagccattccgccttaaccgcccttcagaccgagcaagtacaagattcggagcattcagggaagatggttgcgaaacgccagttcagaatcggcgatatagcgggtgaacatacatcttttgacaagcttcccgaaggcggcagggcgacatatcgcgggacggcattcggttcagacgatgccagtggaaaactgacctacaccatagatttcgccgccaagcagggacacggcaaaatcgaacatttgaaatcgccagaactcaatgttgacctggccgcctccgatatcaagccggataaaaaacgccatgccgtcatcagcggttccgtcctttacaaccaagccgagaaaggcagttactctctaggcatctttggcgggcaagcccaggaagttgccggcagcgcagaagtggaaaccgcaaacggcatacgccatatcggtcttgccgccaagcagtaa

SEQ ID NO: 5 - protein, 279 aa, Artificial Sequence
  Synthetic Construct
MNRTAFCCLFLTTALILTACSSGGGGSGSGGVAADIGTGLADALTAPLDHKDKGLRSLTLDQSVRKNEKLKLAAQGAEKTYGNGDSLNTGKLKNDKVSRFDFIRQIEVDRQLITLESGEFQVYKQSHSALTAFQTEQIQDSEHSGKMVAKRRFRIGDIAGEHTSFDKLPEGGRATYRGTAFSSDDAGGKLTYTIDFAAKQGYGKIEHLKSPELNVDLVSADIKPDEKRHAVISGSVLYNQDEKGSYSLGIFGGKAQEVAGSAEVKTVNGIRHIGLAAKQ

SEQ ID NO: 6 - DNA, 840 bp, Artificial Sequence
  Synthetic Construct
gtgaaccgaactgccttctgctgccttttcctgaccaccgccctgattctgaccgcctgcagcagcggaggcggcggaagcggaagcggcggtgtcgccgccgacatcggcacggggcttgccgatgcgctaaccgcgccgctcgaccataaagacaaaggtttgcggtctttgacgctggaccagtccgtcaggaaaaacgagaaactgaagctggcggcacaaggtgcggaaaaaacttatggaaacggcgacagcctcaatacgggcaaattgaagaacgacaaggtcagccgtttcgactttatccgtcaaatcgaagtggacaggcagctcattaccttggagagcggagagttccaagtgtacaaacaaagccattccgccttaaccgcctttcagaccgagcaaatacaagattcggagcattccgggaagatggttgcgaaacgccggttcagaatcggcgacatagcgggcgaacatacatcttttgacaagcttcccgaaggcggcagggcgacatatcgcgggacggcgttcagttcagacgatgccggcggaaaactgacctacaccatagatttcgccgccaagcagggatacggcaaaatcgaacatttgaaatcgccggaactcaatgtcgacctggtctctgccgatatcaagccggatgaaaaacgccatgccgtcatcagcggctccgtcctttacaaccaagacgagaaaggcagttactccctcggtatctttggcggaaaagcccaggaagttgccggcagcgcggaagtgaaaaccgtaaacggcatacgccatatcggccttgccgccaagcaataa

SEQ ID NO: 7 - protein, 279 aa, Artificial Sequence
  Synthetic Construct
MTRSKPVNRTAFCCLSLTAALILTACSSGGGGVAADIGAGLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPSGKAEYHGKAFSSDDPNGRLHYSIDFTKKQGYGRIEHLKTPEQNVELASAELKADEKSHAVILGDTRYGGEEKGTYHLALFGDRAQEIAGSATVKIREKVHEIGIAGKQ

SEQ ID NO: 8 - DNA, 840 bp, Artificial Sequence
  Synthetic Construct
atgactaggagcaaacctgtgaaccgaactgccttctgctgcctttctttgaccgccgccctgattctgaccgcctgcagcagcggaggcggcggtgtcgccgccgacatcggcgcggggcttgccgatgcactaaccgcaccgctcgaccataaagacaaaagtttgcagtctttgacgctggatcagtccgtcaggaaaaacgagaaactgaagctggcggcacaaggtgcggaaaaaacttatggaaacggcgacagcctcaatacgggcaaattgaagaacgacaaggtcagccgcttcgactttatccgtcaaatcgaagtggacgggcagctcattaccttggagagcggagagttccaaatatacaaacaggaccactccgccgtcgttgccctacagattgaaaaaatcaacaaccccgacaaaatcgacagcctgataaaccaacgctccttccttgtcagcggtttgggtggagaacataccgccttcaaccaactgcccagcggcaaagccgagtatcacggcaaagcattcagctccgacgacccgaacggcaggctgcactactccattgattttaccaaaaaacagggttacggcagaatcgaacacctgaaaacgcccgagcagaatgtcgagcttgcctccgccgaactcaaagcagatgaaaaatcacacgccgtcattttgggcgacacgcgctacggcggcgaagaaaaaggcacttaccacctcgcccttttcggcgaccgcgcccaagaaatcgccggctcggcaaccgtgaagataagggaaaaggttcacgaaatcggcatcgccggcaaacagtag

SEQ ID NO: 9 - protein, 285 aa, Artificial Sequence
  Synthetic Construct
MTRSKPVNRTTFCCLSLTAGPDSDRLQQRRGGGGGVAADIGTGLADALTAPLDHKDKGLKSLTLEASIPQNGTLTLSAQGAEKTFKAGGKDNSLNTGKLKNDKISRFDFVQKIEVDGQTITLASGEFQIYKQDHSAVVALRIEKINNPDKIDSLINQRSFLVSDLGGEHTAFNQLPDGKAEYHGKAFSSDDADGKLTYTIDFAAKQGHGKIEHLKTPEQNVELASAELKADEKSHAVILGDTRYGGEEKGTYRLALFGDRAQEIAGSATVKIGEKVHEIGIADKQ

SEQ ID NO: 10 - DNA, 858 bp, Artificial Sequence
  Synthetic Construct
atgactaggagtaaacctgtgaaccgaactaccttctgctgcctttctttgaccgccggccctgattctgaccgcctgcagcagcggaggggcggaggcggtggtgtcgccgccgacatcggcacggggcttgccgatgcattaaccgcgccgctcgaccataaagacaaaggtttgaaatccctaacattggaagcctccattccccaaaacggaacactgaccctgtcggcacaaggtgcggaaaaaactttcaaagccggcggcaaagacaacagcctcaacacgggcaaactgaagaacgacaaaatcagccgcttcgacttcgtgcaaaaaatcgaagtggacggacaaaccatcacactggcaagcggcgaatttcaaatatacaaacaggatcactccgccgtcgttgccctacggattgaaaaaatcaacaaccccgacaaaatcgacagcctgataaaccaacgctccttccttgtcagcgatttgggcggagaacataccgccttcaaccaactgcctgacggcaaagccgagtatcacggcaaagcattcagctccgacgatgccgacggaaaactgacctataccatagatttcgccgccaaacagggacacggcaaaatcgaacacctgaaaacacccgagcagaatgttgagcttgcctccgccgaactcaaagcagatgaaaaatcacacgccgtcattttgggcgacacgcgctacggcggcgaagagaaaggcacttaccgcctcgcccttttcggcgaccgcgcccaagaaatcgctggctcggcaaccgtgaagataggggaaaaggttcacgaaatcggcatcgccgacaaacagtag

SEQ ID NO: 11 - protein, 187 aa, Artificial Sequence
  Synthetic Construct
MKKIIFAALAAAAISTASAATYKVDEYHANARFAIDHFNTSTNVGGFYGLTGSVEFDQAKRDGKIDITIPIANLQSGSQHFTDHLKSADIFDAAQYPDIRFVSTKFNFNGKKLVSVDGNLTMHGKTAPVKLKAEKFNCYQSPMEKTEVCGGDFSTTIDRTKWGMDYLVNVGMTKSVRIDIQIEAAKQ

SEQ ID NO: 12 - DNA, 564 bp, Artificial Sequence
  Synthetic Construct
atgaaaaaaatcatcttcgccgcactcgcagccgccgccatcagtactgcctccgccgccacctacaaagtggacgaatatcacgccaacgcccgtttcgccatcgaccatttcaacaccagcaccaacgtcggcggtttttacggtctgaccggttccgtcgagttcgaccaagcaaaacgcgacggtaaaatcgacatcaccatccccattgccaacctgcaaagcggttcgcaacactttaccgaccacctgaaatcagccgacatcttcgatgccgcccaatatccggacatccgctttgtttccaccaaattcaacttcaacggcaaaaaactggtttccgttgacggcaacctgaccatgcacggcaaaaccgcccccgtcaaactcaaagccgaaaaattcaactgctaccaaagcccgatggagaaaaccgaagtttgtggcggcgacttcagcaccaccatcgaccgcaccaaatggggcatggactacctcgttaacgttggtatgaccaaaagcgtccgcatcgacatccaaatcgaggcagccaaacaataa

SEQ ID NO: 13 - protein, 187 aa, Artificial Sequence
  Synthetic Construct
MKKIIFAALAAAAVGTASAATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQRPMEKTEVCGGDFSTTIDRTKWGVDYLVNVGMTKSVRIDIQIEAAKQ

SEQ ID NO: 14 - DNA, 564 bp, Artificial Sequence
  Synthetic Construct
atgaaaaaaatcatcttcgccgcactcgcagcggcagccgttggcactgcctccgccgccacctacaaagtggacgaatatcacgccaacgcccgtttcgccatcgaccatttcaacaccagcaccaacgtcggcggtttttacggtctgaccggttccgtcgagttcgaccaagcaaaacgcgacggtaaaatcgacatcaccatccccgttgccaacctgcaaagcggttcgcaacactttaccgaccacctgaaatcagccgacatcttcgatgccgcccaatatccggacatccgctttgtttccaccaaattcaacttcaacggcaaaaaactggtttccgttgacggcaacctgaccatgcacggcaaaaccgcccccgtcaaactcaaagccgaaaaattcaactgctaccaacgcccgatggagaaaaccgaagtttgcggcggcgacttcagcaccaccatcgaccgcaccaaatggggcgtggactacctcgttaacgttggtatgaccaaaagcgtccgcatcgacatccaaatcgaggcagccaaacaataa

SEQ ID NO: 15 - protein, 187 aa, Artificial Sequence
  Synthetic Construct
MKKIIIAALAAAAIGTASAATYKVDEYHANARFSIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMLKTEVCGGDFSTTIDRTKWGMDYLVNVGMTKSVRIDIQIEAAKQ

SEQ ID NO: 16 - DNA, 564 bp, Artificial Sequence
  Synthetic Construct
atgaaaaaaatcatcatcgccgcgctcgcagcagccgccatcggcactgcctccgccgccacctacaaagtggacgaatatcacgccaacgcccgtttctctatcgaccatttcaacaccagcaccaacgtcggcggtttttacggtctgaccggttccgttgagttcgaccaagcaaaacgcgacggtaaaatcgacatcaccatccccgttgccaacctgcaaagcggttcgcaacactttaccgaccacctgaaatcagccgacatcttcgatgccgcccaatatccggacatccgctttgtttccaccaaattcaacttcaacggcaaaaaactggtttccgttgacggcaacctgaccatgcacggcaaaaccgcccccgtcaaactcaaagccgaaaaattcaactgctaccaaagcccgatgttgaaaaccgaagtttgcggcggcgacttcagcaccaccatcgaccgcaccaaatggggcatggactacctcgttaacgttggtatgaccaaaagcgtccgcatcgacatccaaatcgaggcagccaaacaataa

SEQ ID NO: 17 - protein, 187 aa, Artificial Sequence
  Synthetic Construct
MKKIIIAALAAAAVGTASAATYKVDEYHANARFAIDHFNTSTNVGGFYGLTGSVEFDQAKRDGKIDITIPVANLQSGSQHFTDHLKSADIFDAAQYPDIRFVSTKFNFNGKKLVSVDGNLTMHGKTAPVKLKAEKFNCYQSPMAKTEVCGGDFSTSIDRTKWGVDYLVNVGMTKSVRIDIQIEAAKQ

SEQ ID NO: 18 - DNA, 564 bp, Artificial Sequence
  Synthetic Construct
atgaaaaaaatcatcatcgccgcactcgcagcggcagccgtcggcactgcctccgccgccacctacaaagtggacgaatatcacgccaacgcccgtttcgccatcgaccatttcaacaccagcaccaacgtcggcggtttttacggtctgaccggttccgttgagttcgaccaagcaaaacgcgacggtaaaatcgacatcaccatccccgttgccaacctgcaaagcggttcgcaacactttaccgaccacctgaaatcagccgacatcttcgatgccgcccaatatccggacatccgctttgtttccaccaaattcaacttcaacggcaaaaaactggtttccgttgacggcaacctgaccatgcacggcaaaaccgcccccgtcaaactcaaagccgaaaaattcaactgctaccaaagcccgatggcgaaaaccgaagtttgcggcggcgacttcagcaccagcatcgaccgcaccaaatggggcgtggactacctcgttaacgttggtatgaccaaaagcgtccgcatcgacatccaaatcgaggcagccaaacaataa

SEQ ID NO: 19 - protein, 186 aa, Artificial Sequence
  Synthetic Construct
MKKIIFAALAAAAVGTASATYKVDEYHANVRFAIDHFNTSTNVGGFYGLTGSVEFDQAKRDGKIDITIPVANLQSGSQPFTGHLKSADIFDAAQYPDIRFVSTKFNFNGKKLVSVDGNLTMRGKTAPVKLKAEKFNCYQSPMAETEVCGGDFSTTIDRTKWGVDYLVNAGMTKNVRIDIQIEAAKQ

SEQ ID NO: 20 - DNA, 561 bp, Artificial Sequence
  Synthetic Construct
atgaaaaaaatcatcttcgccgcgctcgcagcggcagccgtcggcactgcctccgccacctacaaagtggacgaatatcacgccaacgtccgtttcgccatcgaccacttcaacaccagcaccaacgtcggcggtttttacggtctgaccggttccgtcgagttcgatcaagcaaaacgcgacggcaaaatcgacatcaccattcccgtcgccaacctgcaaagcggttcgcaacccttcaccggccacctgaaatccgccgacatcttcgatgccgctcaatatccggacatccgcttcgtttccaccaaattcaacttcaacggcaaaaaacttgtttccgttgacggcaacctgaccatgcgcggcaaaaccgcccccgtcaaactcaaagccgaaaaattcaactgctaccaaagcccgatggcggaaaccgaagtttgcggcggcgacttcagcaccaccatcgaccgcaccaaatggggcgtggactacctcgttaacgccggtatgaccaaaaacgtccgcatcgacatccaaatcgaagctgcaaaacaataa

SEQ ID NO: 21 - protein, 188 aa, Artificial Sequence
  Synthetic Construct
MRKFVLPLLASLLSVSAAQAADYKLDPTHTKAVFYIDHFNTSTNSGGFYEINGDISFDPETMDGKMDISIPVKTLNTGMAAFDKHVTGADMLDAEKFPTIEFKSTKWNFDEDKLVSVDGDLTMKGKTLPIQLTATKFGCYQSPIFKAEVCGGDFEATIDRTQWGVDFLVKEGMAKMVKLEIQAEAIKQ

SEQ ID NO: 22 - DNA, 567 bp, Artificial Sequence
  Synthetic Construct
atgagaaagttcgtactgcccctcttagcctcgttgctgtcagtctcggctgcccaggctgcggattacaaactggacccaacacatacaaaagctgttttctatattgatcacttcaatacatcgacaaacagcggcggtttctatgaaatcaacggcgatatctcgttcgatcctgaaaccatggatgggaagatggatatttcaattcccgtcaagacgctgaatacaggcatggccgcatttgataagcacgtgacaggtgctgacatgctggacgcggaaaagttcccgaccattgagttcaagtcgaccaagtggaattttgacgaagacaagctggtatcagtggatggcgatctgaccatgaagggaaagacactgccaatccagctcacggccaccaagttcggttgttaccagagcccgatcttcaaggcagaagtctgtggtggcgattttgaagcaactatcgatcgcacccagtggggcgtggacttccttgtaaaagaaggcatggccaagatggtcaagcttgaaattcaggctgaggctatcaagcagtaa

SEQ ID NO: 23 - protein, 191 aa, Artificial Sequence
  Synthetic Construct
MNIMKKNFLGAVLSLGLLSAAHADVYKFDNTHTNAVFNIDHFQTSTNHGGFYAISGELKYQPEKQDAEMRVTIPVSALNTGGDAFDNHIRSSDILDAEKYPEIVFKSTKWHFEDNKPVSIDGLLTMKGVTKPVTLTTTKFGCYMSPIFKAQVCGGDFVTQIDRTQWGVDYLVDMGMTKVVDIKIQAEAVKQ

SEQ ID NO: 24 - DNA, 576 bp, Artificial Sequence
  Synthetic Construct
gtgaatataatgaaaaagaattttcttggtgctgtcctgtctctgggtttattaagtgctgctcatgcagatgtatataaatttgataatacgcatacaaatgcggtatttaatatcgatcatttccagacctcaaccaatcatggcgggttttatgctattagcggcgaactgaaatatcagccggaaaagcaagatgcagagatgcgcgtgacgattcccgtgagcgccttaaataccgggggggatgcgtttgataaccatattcgcagtagcgatattctggatgcggaaaaatatccagaaatagtatttaaatcgacaaaatggcattttgaagataataaaccagtttctattgacggtttgttaacaatgaagggagtaactaagcccgtcaccttaaccaccaccaagtttggctgttatatgagcccgatattcaaggcacaggtttgcggcggtgattttgtcacgcaaatagatcgcacccagtggggtgtagattatctggtcgatatggggatgacgaaagttgtcgatataaaaatccaggcagaagcggttaagcaataa

SEQ ID NO: 25 - protein, 188 aa, Artificial Sequence
  Synthetic Construct
MKKSFLGAALSLGLLSAAHADVYKFDNTHTNAVFNIDHFQTSTNHGGFYAISGELKYQPEKQVAEMRVTIPVSALNTGVDAFDNHIRSSDILDAEKYPEIVFKSTKWHFEDNKPVSIDGLLTMKGVTKPVTLTTTKFGCYMSPIFKAQVCGGDFVTQIDRTQWGVDYLVDMGMTKVVDIKIQAEAVKQ

SEQ ID NO: 26 - DNA, 567 bp, Artificial Sequence
  Synthetic Construct
atgaaaaagagttttcttggtgctgccctgtctctgggtttattaagtgctgctcatgcagatgtatataaatttgataatacgcataccaatgcggtatttaatatcgatcatttccagacctcaaccaatcatggcgggttttatgctattagcggcgaactgaaatatcagccggaaaagcaagtcgcagagatgcgcgtgacgattcccgtaagcgccttaaataccggggtggatgcgtttgataaccatatccgcagtagcgatattctggatgcggaaaaatatccagaaatagtatttaaatcgacaaaatggcattttgaagataataaaccggtttctattgacggtttgttaacaatgaagggggtaactaagcccgtcaccttaaccaccaccaagtttggctgttatatgagcccgatattcaaggcgcaggtttgcggcggtgattttgtcacgcaaatagatcgcacccagtggggggtagattatctggtcgatatggggatgacgaaagttgtcgatataaaaatccaggcagaagcggttaagcaataa

SEQ ID NO: 27 - protein, 191 aa, Artificial Sequence
  Synthetic Construct
MRKSLFALAAAAALVAGAAQAETATYTVEPTHTFATFEISHFGASVNRGRFDKKEGTIALDKAARTGKVDITFQINSINTGTPPFDKHLQSPDIFDAAKYPTARFVGDKFTFDGDKLVSVAGNLTIKGQTHPATFKANQFACYQSPMLKREVCGGDFETTIDRTLFGLDYGVQYGFPKNVRIVAQVEAVKQ

SEQ ID NO: 28 - DNA, 576 bp, Artificial Sequence
  Synthetic Construct
atgcgcaaatccctgttcgccctggccgccgcggccgccctcgttgccggtgccgctcaggcggagaccgccacgtacacggtggagccgacgcacaccttcgccacgttcgagatcagccacttcggcgccagcgtgaaccgcggccgcttcgacaagaaggaaggcacgatcgcgctggacaaggccgccaggaccggcaaggtggacatcaccttccagatcaactccatcaacaccggcacgccgcccttcgacaagcacctgcagagccccgacatcttcgacgcggccaagtaccccaccgcgcgcttcgtgggcgacaagttcaccttcgacggcgacaagctggtgtccgtggccggcaacctgacgatcaagggccagacccatcccgcgaccttcaaggccaaccagttcgcctgctaccagagcccgatgctcaagcgcgaagtgtgcggcggcgatttcgagaccacgatcgaccgcaccctgttcggcctggactacggcgtgcagtacggcttccccaagaacgtgcgcatcgtggcgcaggtcgaggccgtcaagcagtaa

SEQ ID NO: 29 - protein, 191 aa, Artificial Sequence
  Synthetic Construct
MNIMKKSFLGALLSLGLLSAAHADVYKFDNTHTNAVFNIDHFQTSTNHGGFYAISGELKYQPEKQVAEMRVTIPVSALNTGVDAFDNHIRSSDILDAEKYPEMVFKSTKWHFEDNKPVSIDGLLTMKGVTKPVTLTTTKFGCYMSPIFKAQVCGGDFVTQIDRTQWGIDYLVDMGMTKVVDIKIQAEAVKQ

SEQ ID NO: 30 - DNA, 576 bp, Artificial Sequence
  Synthetic Construct
gtgaatataatgaaaaagagttttcttggtgctctcctgtctctgggtttattaagtgctgctcatgcagatgtatataaatttgataatacgcataccaatgcggtatttaatatcgatcatttccagacctcaaccaatcatggcgggttttatgctattagcggcgaactgaaatatcagccggaaaagcaagtcgcagagatgcgtgtgacgattcccgtaagcgccttaaataccggggtggatgcgtttgataaccatatccgcagtagcgatattctggatgcggaaaaatatccagaaatggtatttaaatcaacaaaatggcattttgaagataataaaccggtttctattgacggtttgctaacaatgaagggagtaacgaagcccgtcaccttaaccaccaccaagtttggctgttatatgagcccgatattcaaggcgcaggtttgcggtggtgattttgtcacgcaaatagatcgcacccagtgggggatagattatctggtcgatatggggatgacgaaagttgtcgatataaaaatccaggcagaagcggttaagcaataa

SEQ ID NO: 31 - protein, 191 aa, Artificial Sequence
  Synthetic Construct
MNIMKKSFLGAVLSLGLLSAAHADVYKFDNTHTNAVFNIDHFQTSTNHGGFYAISGELKYQPEKQVAEMRVTIPVSALNTGVDAFDNHIRSSDILDAEKYPEMVFKSTKWHFEDNKPVSIDGLLTMKGVTKPVTLTTTKFGCYMSPIFKAQVCGGDFVTQIDRTQWGVDYLVDMGMTKVVDIKIQAEAVKQ

SEQ ID NO: 32 - DNA, 576 bp, Artificial Sequence
  Synthetic Construct
gtgaatataatgaaaaagagttttcttggtgctgtcctgtctctggggttattaagtgctgctcatgcagatgtatataaatttgataatacgcataccaatgcggtatttaatatcgatcatttccagacctcaaccaatcatggcgggttttatgctattagcggcgaactgaaatatcagccggaaaagcaagtcgcagagatgcgtgtgacgattcccgtgagcgccttaaataccggggtggatgcgtttgataaccatatccgcagtagcgatattctagatgcggaaaaatatccagaaatggtatttaaatcaacaaaatggcattttgaagataataaaccggtttctattgacggtttgctaacaatgaagggagtaacgaagcccgtcaccttaaccaccaccaagtttggctgttatatgagcccgatattcaaggcgcaggtttgcggtggtgattttgtcacgcaaatagatcgcacccagtggggggtagattatctggtcgatatggggatgacgaaagttgtcgatataaaaatccaggcagaagcggttaagcaataa

SEQ ID NO: 33 - protein, 192 aa, Artificial Sequence
  Synthetic Construct
MRKSILTLAAAAALLAGAAHAETATYAMDPTHTFATFEIGHFGTSTNRGRFDKKEGSVQLDRAAKTGKVEVSIDATSVNTGAAAFDKHLQSPDLFDAAKYPTIKFVSDKFSFNGDKVSEIAGNLTLLGKTLPVTLKANQFNCYTSPMLKREVCGGDFETTIDRTAFGMNYGIDWGFPKNVRLVVQVEAVKQQ

SEQ ID NO: 34 - DNA, 579 bp, Artificial Sequence
  Synthetic Construct
atgcgtaaatcaattttgaccctggccgccgcggccgccctgctggccggcgcggcccatgccgaaaccgccacctacgcgatggacccgacgcatacctttgcgacgtttgaaatcggccacttcggcaccagcaccaaccggggccgcttcgacaagaaggaaggctcggtgcaactggaccgcgccgccaagaccggcaaggtggaggtcagcatcgacgccacctccgtcaataccggcgccgccgcgtttgacaagcacctgcaaagccccgacctgttcgacgccgccaaatacccgaccatcaagtttgtctccgacaagttcagcttcaacggcgacaaggtgtccgaaatcgccggcaacctgaccctgcttggcaaaaccctgcccgtcacgctcaaagccaaccagttcaactgctacaccagcccgatgctcaagcgcgaagtgtgcggcggcgacttcgaaaccacgattgaccgcaccgccttcggcatgaactacggcatcgactggggcttcccgaaaaacgtccgcctggtggtccaggtcgaagccgtcaagcagcaataa

SEQ ID NO: 35 - protein, 191 aa, Artificial Sequence
  Synthetic Construct
MNIMKKSFLGALLSLGLLSAAHADVYKFDNTHTNAVFNIDRFQTSTNHGGFYAISGELKYQPEKQVAEMRVTIPVSALNTGVDAFDNHIRSSDILDAEKYPEIVFKSTKWHFEDNKPVSIDGLLTMKGVTKPVTLTTTKFGCYMSPIFKAQVCGGDFVTQIDRTQWGVDYLVDMGMTKVVDIKIQAEAVKQ

SEQ ID NO: 36 - DNA, 576 bp, Artificial Sequence
  Synthetic Construct
gtgaatataatgaaaaagagttttcttggtgctctcctgtctctgggtttattaagtgctgctcatgcagatgtatataaatttgataatacgcataccaatgcggtatttaatatcgatcgtttccagacctcaaccaatcatggcgggttttatgctattagcggcgaactgaaatatcagccggaaaagcaagtcgcagagatgcgtgtgacgattcccgtgagcgccttaaataccggggtggatgcgtttgataaccatattcgcagtagcgatattttggatgcggaaaaatatccagaaatagtatttaaatcgacaaaatggcattttgaagataataaaccagtttctattgacggtttgttaacaatgaagggagtaactaagcccgtcaccttaaccaccaccaagtttggctgttatatgagcccgatattcaaggcgcaggtttgcggtggtgattttgtcacgcaaatagatcgcacccagtggggtgtagattatctggtcgatatggggatgacgaaagttgtcgatataaaaatccaggcagaagcggttaagcaataa

SEQ ID NO: 37 - protein, 191 aa, Artificial Sequence; identical to SEQ ID NO: 35
  Synthetic Construct

SEQ ID NO: 38 - DNA, 576 bp, Artificial Sequence; identical to SEQ ID NO: 36
  Synthetic Construct

SEQ ID NO: 39 - protein, 199 aa, Artificial Sequence
  Synthetic Construct
MNLIQKTLTIATVVGLGSLSVAANAALYEIDPAHANARFSVDHFGTTTNAGGFYGLTGVVDYSPEKKQGFVGITIPMNNLSTNFKPFDKHLKSADFFNVEKYPTAYFKSTKWEFDGDKVKSVKGELTMLDQTHPVTLTATKFNCYDNPILETKTCGGDFETTIDRTQWGINTYTDGGMMKDVKLKIQIEAGLKDDNKKS

SEQ ID NO: 40 - DNA, 600 bp, Artificial Sequence
  Synthetic Construct
gtgaatctaattcaaaaaacgcttactattgccactgtagttggcctaggttctttaagcgttgctgccaatgctgcgctttatgaaattgatcctgcccatgccaatgctcgcttttctgtagaccattttggtaccaccacgaacgctggcggcttctacggtctaacaggtgtggtagattattctcctgagaaaaaacaaggctttgtgggtattaccattcctatgaacaacttaagcaccaactttaagccttttgataagcacttaaaatctgctgacttcttcaacgtagaaaaataccctactgcttacttcaaatctaccaaatgggaatttgatggcgataaagtaaaatcagtcaaaggcgagctaaccatgcttgaccaaacacacccagttactttaacagccactaagtttaactgctacgataacccaatcttagaaacaaaaacttgtggtggtgactttgaaaccactatcgacagaactcagtggggtattaacacttacactgacggcggtatgatgaaagacgttaaactaaaaatccagatcgaagcgggtctaaaagacgataacaaaaagtcttaa

SEQ ID NO: 41 - protein, 202 aa, Artificial Sequence
  Synthetic Construct
MKPKPHTVRTLIAAIFSLALSGCVSAVIGSAAVGAKSAVDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIVTYGNVTYVMGILTPEEQAQITQKVSTTVGVQKVITLYQNYVQR

SEQ ID NO: 42 - DNA, 609 bp, Artificial Sequence
  Synthetic Construct
atgaaacccaaaccgcacaccgtccgcaccctgattgccgccattttcagccttgcccttagcggctgcgtcagcgcagtaatcggaagcgccgccgtcggcgcgaaatccgccgtcgaccgccgaaccaccggcgcgcaaaccgacgacaacgttatggcgttgcgtatcgaaaccaccgcccgttcctatctgcgccaaaacaaccaaaccaaaggctacacgccccaaatctccgtcgtcggctacaaccgccacctgctgctgctcggacaagtcgccaccgaaggcgaaaaacagttcgtcggtcagattgcacgttccgaacaggccgccgaaggcgtgtacaactatattaccgtcgcctccctgccgcgcactgccggcgacatcgccggcgacacttggaacacatccaaagtccgcgccacgctgttgggcatcagccccgccacacaggcgcgcgtcaaaatcgttacctacggcaacgtaacctacgttatgggcatcctcacccccgaagaacaggcgcagattacccaaaaagtcagcaccaccgtcggcgtacaaaaagtcatcaccctctaccaaaactacgtccaacgctga

SEQ ID NO: 43 - protein, 202 aa, Artificial Sequence; identical to SEQ ID NO: 41
  Synthetic Construct

SEQ ID NO: 44 - DNA, 609 bp, Artificial Sequence
  Synthetic Construct
atgaaacccaaaccgcacaccgtccgcaccctgattgccgccattttcagccttgcccttagcggctgcgtcagcgcagtaatcggaagcgccgccgtcggcgcgaaatccgccgtcgaccgccgcaccaccggcgcgcaaaccgacgacaacgtaatggcgttgcgtattgaaaccaccgcccgctcctacctacgccaaaacaaccaaaccaagggctacacgccccaaatctccgtcgtcggctacaaccgccacctgctgctgctcggacaagtcgccaccgaaggcgaaaaacagttcgtcggtcagattgcacgttccgaacaggccgccgaaggcgtgtacaactacattaccgtcgcctccctgccgcgcactgccggcgacatcgccggcgacacttggaacacatccaaagtccgcgccacgctgttgggcatcagccccgccacacaggcgcgcgtcaaaatcgttacctacggcaacgtaacctacgttatgggcatcctcacccccgaagaacaggcgcagattacccaaaaagtcagcaccaccgtcggcgtacaaaaagtcatcaccctctaccaaaactacgtccaacgctga

SEQ ID NO: 45 - protein, 202 aa, Artificial Sequence; identical to SEQ ID NO: 41
  Synthetic Construct

SEQ ID NO: 46 - DNA, 609 bp, Artificial Sequence
  Synthetic Construct
atgaaacccaaaccgcacaccgtccgcaccctgattgccgccattttcagccttgcccttagcggctgcgtcagcgcagtaatcggaagcgccgccgtcggcgcgaaatccgccgtcgaccgccgaaccaccggcgcgcaaaccgacgacaacgttatggcgttgcgtatcgaaaccaccgcccgctcctatctgcgccaaaacaaccaaaccaaaggctacacgccccaaatctccgttgtcggctacaaccgccacctgctgctgctcggacaagtcgccaccgaaggcgagaaacagttcgtcggtcagattgcacgttccgaacaggccgccgaaggcgtatacaactacattaccgtcgcctccctgccgcgcactgccggcgacatcgccggcgacacttggaacacatccaaagtccgcgccacgctgttgggcatcagccccgccacacaggcgcgcgtcaaaatcgttacctacggcaacgtaacctacgttatgggcatcctcacccccgaagaacaggcgcagattacccaaaaagtcagcaccaccgtcggcgtacaaaaagtcatcaccctctaccaaaactacgtccaacgctga

SEQ ID NO: 47 - protein, 202 aa, Artificial Sequence
  Synthetic Construct
MKPKPHTVRTLTAAVLSLALGGCVSAVVGGAAVGAKSAVDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIVTYGNVTYVMGILTPEEQAQITQKVSTTVGVQKVITLYQNYVQR

SEQ ID NO: 48 - DNA, 609 bp, Artificial Sequence
  Synthetic Construct
atgaaacccaaaccgcacaccgtccgcaccctgactgccgccgtcctcagccttgccctcggcggctgcgtcagcgcagtcgtcggcggcgcggcggtcggcgcgaaatccgccgtcgaccgccgaaccaccggcgcgcaaaccgacgacaacgtaatggcgctgcgtatcgaaaccaccgcccgctcctatctgcgccaaaacaaccaaaccaaaggctacacgccccaaatctccgttgtcggctacaaccgccacctgctgctgctcggacaagtcgccaccgaaggcgagaaacagttcgtcggtcagattgcacgttccgaacaggccgccgaaggcgtgtacaactacattaccgtcgcctccctgccgcgcactgccggcgacatcgccggcgacacttggaacacatccaaagtccgcgccacgctgttgggcatcagccccgccacacaggcgcgcgtcaaaatcgttacctacggcaacgtaacctacgttatgggcatcctcacccccgaagaacaggcgcagattacccaaaaagtcagcaccaccgtcggcgtacaaaaagtcatcaccctctaccaaaactacgtccaacgctga

SEQ ID NO: 49 - protein, 202 aa, Artificial Sequence
  Synthetic Construct
MKPKPHTVRTLIAAVLSLALGGCFSAVVGGAAVGAKSVIDRRTTGAQTDDNVMALRIETTARSYLRQNNQTKGYTPQISVVGYNRHLLLLGQVATEGEKQFVGQIARSEQAAEGVYNYITVASLPRTAGDIAGDTWNTSKVRATLLGISPATQARVKIITYGNVTYVMGILTPEEQAQITQKVSTTVGVQKVITLYQNYVQR

SEQ ID NO: 50 - DNA, 609 bp, Artificial Sequence
  Synthetic Construct
atgaaacccaaaccacacaccgtccgcaccctgattgccgccgtcctcagccttgccctcggcggctgcttcagcgcagtcgtcggcggggccgccgtcggcgcaaaatccgtcatcgaccgccgaaccaccggcgcgcaaaccgatgacaacgttatggcgttgcgtatcgaaaccaccgcccgttcctacctgcgccaaaacaaccaaaccaaaggctacacgccccaaatctccgtcgtcggctacaaccgccacctgctgctgctcggacaagtcgccaccgaaggcgaaaaacagttcgtcggtcagattgcacgttccgaacaggccgccgaaggcgtatacaactacattaccgtcgcctccctgccgcgcactgcgggcgacatcgccggcgacacttggaacacgtccaaagtccgcgccacgctgctgggcatcagccccgctacacaggcgcgcgtcaaaatcattacctacggcaatgtaacctacgttatgggcatcctcacccccgaagaacaggcgcagattacccaaaaagtcagcaccaccgtcggcgtacaaaaagtcattaccctctaccaaaactacgtccaacgctga

SEQ ID NO: 51 - protein, 428 aa, Artificial Sequence
  Synthetic Construct
MIYIVLFLAVVLAVVAYNMYQENQYRKKVRDQFGHSDKDALLNSKTSHVRDGKPSGGSVMMPKPQPAVKKTAKPQDPAMRNLQEQDAVYIAKQKQAKASPFKTEIETALEESGIIGNSAHTVSEPQTGHSAPKPADAPAKPAPVPQTPAKPLITLKELSKVELPWFDVRFDFISYIALTEAKELHALPRLSNRCRYQIVGCTMDDHFQIAEPIPGIRYQAFIVGIQAVSRNGLASQEELSAFNRQVDAFAQSMGGQTLHTDLAAFIEVASALDAFCARVDQTIAIHLVSPTSISGVELRSAVTGVGFVLEDDGAFHYTDTSGSTMFSICSLNNEPFTNALLDNQSYKGFSMLLDIPHSPAGEKTFDDLFMDLAVRLSGQLNLNLVNDKMEEVSTQWLKDVRTYVLARQSEMLKVGIEPGGKTALRLFS

SEQ ID NO: 52 - DNA, 1287 bp, Artificial Sequence
  Synthetic Construct
atgatttacatcgtactgtttctagctgtcgtcctcgccgttgtcgcctacaacatgtatcaggaaaaccaataccgcaaaaaagtgcgcgaccagttcggacactccgacaaagatgccctgctcaacagcaaaaccagccatgtccgcgacggcaaaccgtccggcgggtcagtcatgatgccgaaaccccaaccggcggtcaaaaaaacggcaaaaccccaagaccccgccatgcgcaacctgcaagaacaggatgccgtctacatcgccaagcagaaacaggcaaaagcctccccgttcaaaaccgaaatcgaaaccgccttggaagaaagcggcattatcggcaactccgcccacaccgtttccgaaccccaaaccggacattccgcaccgaaacctgccgacgcgccggcaaaacctgcacccgttccgcaaacacctgcaaaaccgctgattacgctcaaagaactgtcaaaagtcgaattaccctggtttgacgtgcgcttcgacttcatctcctatatcgcgctgaccgaagccaaagaactgcacgcactgccgcgcctttccaaccgctgccgctaccagattgtcggctgcaccatggacgaccatttccagattgccgaacccatcccgggcatccgctatcaggcatttatcgtgggtattcaggcagtcagccgcaacggacttgcctcgcaggaagaactctccgcattcaaccgccaggtggacgcattcgcacaaagcatgggcggtcagacgctgcacaccgaccttgccgcctttatcgaagtggcttccgcactggacgcattctgcgcgcgcgtcgaccagaccatcgccatccatttggtttccccgaccagcatcagcggcgtagaactgcgttccgccgtaacgggcgtgggtttcgttttggaagacgacggcgcgttccactataccgacacgtcgggctcgaccatgttctccatctgctcgctcaacaacgagccgtttaccaacgcccttttggacaaccagtcctacaaaggcttcagtatgctgctcgacatcccgcactctccggcaggcgaaaaaaccttcgacgatttgtttatggatttggcggtacgcctgtccggccagttgaacctgaatctggtcaacgacaaaatggaagaagtttcgacccaatggctcaaagacgtgcgcacttatgtattggcgcgtcagtccgagatgctcaaagtcggtatcgaaccgggcggcaaaaccgcattgcgcctgttctcctaa

SEQ ID NO: 53 - protein, 428 aa, Artificial Sequence; identical to SEQ ID NO: 51
  Synthetic Construct

SEQ ID NO: 54 - DNA, 1287 bp, Artificial Sequence
  Synthetic Construct
atgatttacatcgtactgtttctagctgtcgtcctcgccgttgtcgcctacaacatgtatcaggaaaaccaataccgcaaaaaagtgcgcgaccagttcggacactccgacaaagatgccctgctcaacagcaaaaccagccatgtccgcgacggcaaaccgtccggcgggtcagtcatgatgccgaaaccccaaccggcggtcaaaaaaacggcaaaaccccaagaccccgccatgcgcaacctgcaagaacaggatgccgtctacatcgccaagcagaaacaggcaaaagcctccccgttcaaaaccgaaatcgaaaccgccttggaagaaagcggcattatcggcaactccgcccacaccgtttccgaaccccaaaccggacattccgcaccgaaacctgccgacgcgccggcaaaacctgcacccgttccgcaaacacctgcaaaaccgctgattacgctcaaagaactgtcaaaagtcgaattaccctggtttgacgtgcgcttcgacttcatctcctatatcgcgctgaccgaagccaaagaactgcacgcactgccgcgcctttccaaccgctgccgctaccagattgtcggctgcaccatggacgaccatttccagattgccgaacccatcccgggcatccgctatcaggcatttatcgtgggtattcaggcagtcagccgcaacggacttgcctcgcaggaagaactctccgcattcaaccgccaggtggacgcattcgcacaaagcatgggcggtcagacgctgcacaccgaccttgccgcctttatcgaagtggcttccgcactggacgcattctgcgcgcgcgtcgaccagaccatcgccatccatttggtttccccgaccagcatcagcggcgtagaactgcgttccgccgtaacgggcgtgggtttcgttttggaagacgacggcgcgttccactataccgacacgtcgggctcgaccatgttctccatctgctcgctcaacaacgagccgtttaccaatgcccttttggacaaccagtcctacaaaggcttcagtatgctgctcgacatcccgcactctccggcaggcgaaaaaaccttcgacgatttgtttatggatttggcggtacgcctgtccggccagttgaacctgaatctggtcaacgacaaaatggaagaagtttcgacccaatggctcaaagacgtgcgcacttatgtattggctcgtcagtccgagatgctcaaagtcggtatcgaaccgggcggcaaaaccgcattgcgcctgttctcctaa

SEQ ID NO: 55 - protein, 431 aa, Artificial Sequence
  Synthetic Construct
MIAMIYIVLFLAAVLAVVAYNMYQENQYRKKVRDQFGHSDKDALLNSKTSHVRDGKPSGGPVMMPKPQPAVKKTAKPQDPAMRNLQEQDAVYIAKQKQAKASPFKTEIETALEESGIIGNSAHTVSEPQTGHSAPKPADAPAKPVPVPQTPAKPLITLKELSKVELPWFDVRFDFISYIALTEAKELHALPRLSNRCRYQIVGCTMDDHFQIAEPIPGIRYQAFIVGIQAVSRNGLASQEELSAFNRQVDAFAQSMGGQTLHTDLAAFIEVASALDAFCARVDQTIAIHLVSPTSISGVELRSAVTGVGFVLEDDGAFHYTDTSGSTMFSICSLNNEPFTNALLDNQSYKGFSMLLDIPHSPAGEKTFDDLFMDLAVRLSGQLNLNLVNDKMEEVSTQWLKDVRTYVLARQSEMLKVGIEPGGKTALRLFS

SEQ ID NO: 56 - DNA, 1296 bp, Artificial Sequence
  Synthetic Construct
gtgattgccatgatttacatcgtactgttcctcgccgccgtcctcgccgttgtcgcctacaatatgtatcaggaaaaccaataccgcaaaaaagtgcgcgaccagttcgggcactccgacaaagatgccctgctcaacagcaaaaccagccatgtccgcgacggcaaaccgtccggcgggccagtcatgatgccgaaaccccaaccggcggtcaaaaaaacggcaaaaccccaagaccccgccatgcgcaacctgcaagaacaggatgccgtctacatcgccaagcagaaacaggcaaaagcctccccgttcaaaaccgaaatcgaaaccgccttggaagaaagcggcattatcggcaactccgcccacaccgtttccgaaccccaaaccggacattccgcaccgaaacctgccgacgcgccggcaaaacccgttcccgttccgcaaacgccggcaaaaccgctgattacgctcaaagagctgtcgaaggtcgagctgccctggtttgacgtgcgcttcgacttcatctcttatatcgcgctgaccgaagccaaagaacttcacgcactgccgcgcctttccaaccgctgccgctaccagattgtcggctgcaccatggacgaccatttccagattgccgaacccatcccgggcatccgctatcaggcatttatcgtgggtattcaggcagtcagccgcaacggacttgcctcgcaggaagaactctccgcattcaaccgccaggtggacgcattcgcacaaagcatgggcggtcagacgctgcacaccgaccttgccgcctttatcgaagtggcttccgcactggacgcattctgcgcgcgcgtcgaccagaccatcgccatccatttggtttccccgaccagcatcagcggcgtagaactgcgttccgccgtaacgggcgtgggtttcgttttggaagacgacggcgcgttccactataccgacacgtcgggctcgaccatgttctccatctgctcgctcaacaacgagccgtttaccaatgcccttttggacaaccagtcctacaaaggcttcagtatgctgctcgacatcccgcactctccggcaggcgaaaaaaccttcgacgatttgtttatggatttggcggtacgcctgtccggccagttgaacctgaatctggtcaacgacaaaatggaagaagtttcgacccaatggctcaaagacgtgcgcacttatgtattggctcgtcagtccgagatgctcaaagtcggtatcgaaccgggcggcaaaaccgcattgcgcctgttctcctaa

SEQ ID NO: 57 - protein, 428 aa, Artificial Sequence
  Synthetic Construct
MIYIVLFLAAVLAVVAYNMYQENQYRKKVRDQFGHSDKDALLNSKTSHVRDGKPSGGPVMMPKPQPAVKKTAKSQDPAMRNLQEQDAVYIAKQKQAKASPFKTEIETALEESGIIGNSAHTVPEPQTGHSAPKPADAPAKPVPVPQTPAKPLITLKELSKVELPWFDVRFDFISYIALTEAKELHALPRLSNRCRYQIVGCTMDDHFQIAEPIPGIRYQAFIVGIQAVSRNGLASQEELSAFNRQVDAFAHSMGGQTLHTDLAAFIEVASALDAFCARVDQTIAIHLVSPTSISGVELRSAVTGVGFVLEDDGAFHYTDTSGSTMFSICSLNNEPFTNALLDNQSYKGFSMLLDIPHSPAGEKTFDDLFMDLAVRLSGQLNLNLVNDKMEEVSTQWLKDVRTYVLARQSEMLKVGIEPGGKTALRLFS

SEQ ID NO: 58 - DNA, 1287 bp, Artificial Sequence
  Synthetic Construct
atgatttacatcgtactgttcctcgccgccgtcctcgccgttgtcgcctacaatatgtatcaggaaaaccaataccgcaaaaaagtgcgcgaccagttcgggcactccgacaaagatgccctgctcaacagcaaaaccagccatgtccgcgacggcaaaccgtccggcgggccagtcatgatgccgaaaccccaaccggcggtcaaaaaaacggcaaaatcccaagaccccgccatgcgcaacctgcaagagcaggatgccgtctacatcgccaagcagaaacaggcaaaagcctccccgttcaaaaccgaaatcgaaaccgccttggaagaaagcggcattatcggcaactccgcccacaccgttcccgaaccccaaaccggacattccgcaccaaaacctgccgacgcgccggcaaaacctgttcccgttccgcaaacgccggcaaaaccgctgattacgctcaaagagctgtcgaaggtcgagctgccctggtttgacgtgcgcttcgacttcatctcttatatcgcgctgaccgaagccaaagaactgcacgcactgccgcgcctttccaaccgctgccgctaccagattgtcggctgcaccatggacgaccatttccagattgccgaacccatcccgggcatccgctatcaggcatttatcgtgggtattcaggcagtcagccgcaacggacttgcctcgcaggaagaactctccgcattcaaccgccaggtggatgcattcgcacacagcatgggcggtcagacgctgcacaccgaccttgccgcctttatcgaagtggcttccgcactggacgcattctgcgcgcgcgtcgaccagactatcgccatccatttggtttccccgaccagcatcagcggcgtagaactgcgttccgccgtaacgggcgtgggtttcgttttggaagacgacggcgcgttccactataccgacacgtcgggctcgaccatgttctccatctgctcgctcaacaacgagccgtttaccaatgcccttttggacaaccagtcctataaaggcttcagtatgctgctcgacatcccgcactctccggcaggcgaaaaaaccttcgacgatttgtttatggatttggcggtacgcctgtccggccagttgaacctgaatctggtcaacgacaaaatggaagaagtttcgacccaatggctcaaagacgtgcgcacttatgtattggctcgtcagtccgagatgctcaaagtcggtatcgaaccgggcggcaaaaccgcattgcgcctgttctcctaa

SEQ ID NO: 59 - protein, 428 aa, Artificial Sequence
  Synthetic Construct
MIYIVLFLAAVLAVVAYNMYQENQYRKKVRDQFGHSDKDALLNSKTSHVRDGKPSGGPVMMPKPQPAVKKPAKPQDSAMRNLQEQDAVYIAKQKQAKASPFKTEIETALEEIGIIGNSAHTVSEPQTGHSAPKPADAPAKPVPVPQTPAKPLITLKELSKVELPWFDVRFDFISYIALTEAKELHALPRLSNRCRYQIVGCTMDDHFQIAEPIPGIRYQAFIVGIQAVSRNGLASQEELSAFNRQADAFAQSMGGQTLHTDLAAFIEVASALDAFCARVDQTIAIHLVSPTSISGVELRSAVTGVGFVLEDDGAFHYTDTSGSTMFSICSLNNEPFTNALLDNQSYKGFSMLLDIPHSPAGEKTFDDLFMDLAVRLSGQLNLNLVNDKMEEVSTQWLKDVRTYVLARQSEMLKVGIEPGGKTALRLFS

SEQ ID NO: 60 - DNA, 1287 bp, Artificial Sequence
  Synthetic Construct
atgatttacatcgtactgttcctcgccgccgtcctcgccgttgtcgcctacaatatgtatcaggaaaaccaataccgcaaaaaagtgcgcgaccagttcggacactccgacaaagatgccctgctcaacagcaaaaccagccatgtccgcgacggcaaaccgtccggcgggccagtcatgatgccgaaaccccaaccggcggtcaaaaaaccggccaaaccccaagactccgccatgcgcaacctgcaagaacaggatgccgtctacatcgccaagcagaaacaggcaaaagcctccccgttcaaaaccgaaatcgaaaccgccttggaagaaatcggcattatcggcaactccgcccacaccgtttccgaaccccaaaccggacattccgcaccgaaacctgccgacgcgccggcaaaacccgttcccgttccgcaaacgccggcaaaaccgctgattacgctcaaagagctgtcgaaggtcgagctgccctggtttgacgtgcgcttcgacttcatctcctatatcgcgctgaccgaagccaaagaactgcacgcactgccgcgcctttccaaccgctgccgctaccagattgtcggctgcaccatggacgaccatttccagattgccgaacccatcccgggcatccgctatcaggcatttatcgtgggtatccaggcagtcagccgcaacggacttgcctcgcaggaagaactctccgcattcaaccgccaggcggacgcattcgcacaaagcatgggcggtcagacgctgcacaccgaccttgccgcctttatcgaagtggcttccgcactggacgcattctgcgcgcgcgtcgaccagaccatcgccatccatttggtttcgccgaccagcatcagcggcgtagaactgcgttccgccgtaacgggcgtgggtttcgttttggaagacgacggcgcgttccactataccgacacgtcgggctcgaccatgttctccatctgctcgctcaacaacgagccgtttaccaatgcccttttggacaaccagtcctacaaaggcttcagtatgctgctcgacatcccgcactctccggcaggcgaaaaaaccttcgacgatttgtttatggatttggcggtacgcctgtccggtcagttgaacctgaatctggtcaacgacaaaatggaagaagtttcgacccaatggctcaaagacgtacgcacttatgtattggcgcgtcagtccgagatgctcaaagtcggtatcgaaccgggcggcaaaaccgccctgcgcctgttttcataa

SEQ ID NO: 61 - protein, 1158 aa, Artificial Sequence
  Synthetic Construct
MFKSNYERKMRYSIRKFSVGVASVAVASLFMGSVAHASELVKDDSVKTTEVAAKPYPSMAQTDQGNNSSSSELETTKMEIPTTDIKKAVEPVEKTAGETSATDTGKREKQLQQWKNNLKNDVDNTILSHEQKNEFKTKIDETNDSDALLELENQFNETNRLLHIKQHEEVEKDKKAKQQKTLKQSDTKVDLSNIDKELNHQKSQVEKMAEQKGITNEDKDSMLKKIEDIRKQAQQADKKEDAEVKVREELGKLFSSTKAGLDQEIQEHVKKETSSEENTQKVDEHYVNSLQNLAQKSLEELDKATTNEQATQVKNQFLENAQKLKEIQPLIKETNVKLYKAMSESLEQVEKELKHNSEANLEDLVAKSKEIVREYEGKLNQSKNLPELKQLEEEAHSKLKQVVEDFRKKFKTSEQVTPKKRVKRDLAANENNQQKIELTVSPENITVYEGEDVKFTVTAKSDSKTALDFSDLLTKYNPSVSDRISTNYKTNTDNHKIAEITIKNLKLNESQTVTLKAKDDSGNVVEKTFTITVQKKEEKQVPKTPEQKDSKTEEKVPQEPKSNDKNQLQELIKSAQQELEKLEKAIKELMEQPEIPSNPEYGIQKSIWESQKEPIQEAITSFKKIIGDSSSKYYTEHYFNKYKSDFMNYPLHAQMEMLTRKVVQYMNKYPDNAEIKKIFESDMKRTKEDNYGSLENDALKGYFEKYFLTPFNKIKQIVDDLDKKVEQDQPAPIPENSEMDQAKEKAKIAVSKYMSKVLDGVHQHLQKKNHSKIVDLFKELEAIKQQTIFDIDNAKTEVEIDNLVHDAFSKMNATVAKFQKGLETNTPETPDTPKIPELPQAPDTPQAPDTPHVPESPKAPEAPRVPESPKTPEAPHVPESPKAPEAPRVPESPKTPEAPHVPESPKTPEAPKIPEPPKTPDVPKLPDVPKLPDAPKLPDGLNKVGQAVFTSTDGNTKVTVVFDKPTDADKLHLKEVTTKELADKIAHKTGGGTVRVFDLSLSKGGKETHVNGERTVRLALGQTGSDVHVYHVKENGDLERIPSKVENGQVVFKTNHFSLFAIKTLSKDQNVTPPKQTKPSTQGSQVEIAESQTGKFQSKAANHKALATGNETVAKGNPTSTTEKKLPYTGVASNLVLEIMGLLGLIGTSFIAMKRRKS

SEQ ID NO: 62 - DNA, 3477 bp, Artificial Sequence
  Synthetic Construct
tcatgattttcttcttttcattgcgatgaatgaagttccaatcaaaccaaggagacccataatttcaagaactagattagatgccactcctgtatatggcaatttcttttccgttgttgatgtaggatttccttttgccactgtttcatttccagtagccagtgctttatgattagctgctttactctggaattttccagtttgactctctgcaatctctacttgactgccttgggtagaaggtttagtctgcttcggtggagtaacattttgatccttagaaagtgtcttaatcgcaaacaaactgaagtggttcgttttaaaaacaacttgcccattttcaactttagaaggaatacgctcaaggtcgccattttcctttacgtgatagacgtgaacatctgagccagtctgcccaagcgcgagccgaacagttcgttctccattgacatgtgtttccttgcctcctttagaaagagataagtcaaacacacgaactgttcctcctcctgttttatgagcaattttatcagccaactctttcgtcgttacttccttgagatgtaacttatcagcatctgtaggtttatcaaatacaaccgtaaccttagtatttccatcagttgatgtaaatactgcttgtccaactttatttaacccatctggtaacttcggtgcatctggaagcttagggacgtctggaagcttagggacgtctggagtcttagggggttccggaatctttggtgcttctggagtctttggtgattccggaacatgcggtgcttctggagtctttggtgattccggaacacgcggtgcttctggggcctttggtgattccggaacatgcggtgcttctggagtctttggtgattccggaacacgcggtgcttctggggcctttggtgattccggaacatgcggtgtgtctggagcctgcggtgtatctggggcttgaggtagctctggaatcttcggtgtatctggagtttctggcgtatttgtctctagacctttttgaaatttagcaacagtagcattcatttttgagaatgcatcgtgtactaagttatcaatctctacttcagtctttgcattgtcaatatcaaaaatagtttgttgtttaatcgcttcaagttccttaaaaagatcaacaattttactgtgatttttcttctgcagatgttgatgaactccatctaaaaccttactcatatacttcgatacagcaatcttagccttttccttagcctgatccatttctgaattttccggaattggtgctggctgatcttgttctacttttttatccaaatcatctacaatctgcttaattttattaaatggtgtaaggaaatatttctcaaaatagcctttcaaagcatcattttctaaacttccgtaattatcttctttcgttctcttcatatctgactcaaatatctttttaatttctgcattatcaggatatttgttcatatactgaaccacttttctagtcagcatctccatttgtgcatgaagtggataattcataaaatcagatttatatttgttaaaatagtgctctgtgtagtattttgaagatgaatcaccaataatcttcttaaaacttgttatggcttcctggataggctctttttgtgactcccaaatagatttttgaataccatactctggattggatggaatctctggttgctccattaattcttttattgctttttctaacttttccagttcttgttgagctgatttaatcaactcttgtaattgattcttgtcatttgattttggttcttgaggaaccttttcttccgttttagaatctttctgctctggtgttttaggaacttgtttctcctctttcttttgcactgtaatagtgaatgttttttcaactacattgccagaatcatctttagcttttagagtcactgtttgactttcatttagcttcaaattcttgatagtgatttcggcaatcttatgattatccgtgttagtcttataatttgtactaattctatctgatacagacggattatattttgttaaaagatcactgaagtccaacgccgtcttcgaatcacttttagctgtgactgtaaatttcacgtcttcaccttcatatacagtgatattctctggtgaaactgttaactcaatcttttgttgattattttcattagcagctaaatctcgtttgacacgtttttttggtgtcacttgctctgacgttttaaatttttttctaaaatcctccacaacttgtttcaacttcgaatgagcttcctcttctagttgctttaattctggaagatttttagattgattaagttttccttcgtattctcttacgatttctttagatttcgcaaccaaatcttctaaattagcttccgaattatgttttaattccttctcaacctgctccaagctctcactcatagccttatacaatttcacattcgtttctttgataagaggttgtatttctttgagcttttgagcgttttctaagaattgatttttaacttgtgtagcttgttcattggtagttgccttatctagttcttcaagagatttttgagcaaggttctgaaggctattaacatagtgttcatcaactttctgagtattttcctcactactcgtttctttcttcacatgctcttgaatttcttgatccagaccagctttagttgaactaaagagtttacctagttcttcacgaacctttacttcggcatcttcttttttatctgcttgttgagcttgtttacgaatatcttcgatttttttcagcatagaatctttatcttcatttgtgattcccttttgctctgccattttttcaacttgacttttttgatgattaagctctttgtcaatattgcttagatctactttcgtatctgactgtttcagagttttctgttgcttagctttcttatctttctcaacttcttcatgttgtttgatgtgtaacagtctattagtttcgttaaattgattttctaattctaataatgcatcagaatcatttgtttcatcaatttttgttttaaactcatttttctgttcatgagatagaattgtgttatccacatcattttttagattatttttccattgttgtaattgtttctctcgttttccagtatcagtggcagatgtttccccagctgttttctcgaccggttcaacagctttttttatgtctgttgtaggaatttccatctttgttgtctcaagttccgaggatgatgaattatttccttgatctgtttgagccatacttggatagggcttagctgcaacctcggtagtcttcacactatcgtcctttacaagctcacttgcatgagcaacgcttcccatgaacaaactagctaccgctacactagctactcctacactaaatttacgaatggaataacgcatttttctttcataattagatttaaacat

SEQ ID NO: 63 - protein, 185 aa, Artificial Sequence
  Synthetic Construct
MQLTKKYLPAILLLLSLASCDLFYKNRNSNANLLKTLDNNQKQALIYFKDTLQDKKYLSYLTTSQKNFLDDLEKNKKAPGLQYKLKKTLSSEYDESQFNKLLNELGNAKAKQFLQQLHIMLQSIKDGTLTSFSSANFNDLQNLEQKKERALQSINGELYVEYYFYINGISNPDNFFEKIMQNLKT

SEQ ID NO: 64 - DNA, 558 bp, Artificial Sequence
  Synthetic Construct
atgcaattaacaaaaaaatatttaccagcaattcttttattattaagccttgctagctgtgatttattctataaaaacagaaactcaaatgctaatttactaaaaactcttgataataatcaaaaacaagctctcatttactttaaagatacacttcaagataaaaaatatttaagttatttaacgacaagccagaaaaattttttagacgatttagagaaaaataaaaaagctcctggtttgcaatataaacttaaaaaaacactaagctctgagtatgatgaaagtcaattcaataaattacttaatgaacttggaaatgctaaggctaaacaattccttcaacaattgcatataatgctacagtctatcaaagacggcacgcttacaagcttttcatctgcaaatttcaatgacttgcaaaatttagagcaaaaaaaagaacgagcattgcaatctatcaatggtgaattatatgttgaatactatttctatatcaatggaattagcaatccggacaatttttttgaaaaaataatgcaaaatttaaaaacttaa

SEQ ID NO: 65 - protein, 240 aa, Artificial Sequence
  Synthetic Construct
MKKSFLSIYMLISISLLSCDVSRLNQRNIDELKIFVEKAKYYSIKLDAIYSEYTGAYNDIMTYIMTYSEGTSSDKSKVNQAISILKKDNKIVNKFKELEKIIEEYKPMFLSKLIDDFAIELDQAVDNDVSNARHVADSYEKLRKSVALAYIESFDVISSKFVDSKFVEASKKFVNKAKEFVEENDLIALKCIVKTIGDMVNDREINSRSRYNNFYKKEADFLGAAVELEGAYKAIKQTLL

SEQ ID NO: 66 - DNA, 779 bp, Artificial Sequence
  Synthetic Construct
ctatgaatctctaaagattttagcaggagagaaaatatgaaaaaaagttttttatcaatatacatgttaatttcaataagtttattatcatgtgatgttagtagattaaatcagagaaatattgatgagcttaaaatttttgttgaaaaggccaagtattattctataaaattagacgctatttatagcgaatatacaggagcatataatgatattatgacttatattatgacttattcggaaggtacatcttctgataaaagtaaggttaatcaagctatatctatacttaaaaaagacaataaaattgttaataagtttaaggagcttgaaaagattatagaagaatacaaacccatgtttttaagtaaattaattgatgattttgctatagaattagaccaagctgtagataatgatgtgtctaatgccagacatgttgctgattcttatgaaaaacttagaaaatctgttgcattagcctacattgagagttttgatgttatatcttctaagtttgttgatagtaagtttgttgaagcttctaaaaaatttgtcaataaagctaaagagtttgtagaggaaaatgatcttatagctcttaagtgtattgtgaaaactattggagatatggttaatgatagggaaataaattcaagaagcaggtataataatttttataaaaaagaagcagattttttaggtgctgctgtagaacttgagggggcttataaagctattaagcaaactttattatagatcaaggtataaattttagg

SEQ ID NO: 67 - protein, 492 aa, Artificial Sequence
  Synthetic Construct
MARENTNKHYSLRKLKKGTASVAVALSVLGAGLVVNTNEVSAAVTRGTINDPQRAKEALDKYELENHDLKTKNEGLKTENEGLKTENEGLKTENEGLKTEKKEHEAENDKLKQQRDTLSTQKETLEREVQNTQYNNETLKIKNGDLTKELNKTRQELANKQQESKENEKALNELLEKTVKDKIAKEQENKETIGTLKKILDETVKDKIAKEQENKETIGTLKKILDETVKDKLAKEQKSKQNIGALKQELAKKDEANKISDASRKGLRRDLDASREAKKQLEAEHQKLEEQNKISEASRKGLRRDLDASREAKKQLEAEHQKLEEQNKISEASRKGLRRDLDASREAKKQVEKALEEANSKLAALEKLNKELEESKKLTEKEKAELQAKLEAEAKALKEQLAKQAEELAKLRAGKASDSQTPDTKPGNKAVPGKGQAPQAGTKPNQNKAPMKETKRQLPSTGETANPFFTAAALTVMATAGVAAVVKRKEEN

SEQ ID NO: 68 - DNA, 1479 bp, Artificial Sequence
  Synthetic Construct
atggctagagaaaataccaataagcattattcgcttagaaaattaaaaaaaggcactgcatcagtagcagtagctttgagtgtcttaggagcaggattagttgtcaatactaatgaagttagtgcagccgtgactaggggtacaataaatgacccgcaaagagcaaaagaagctcttgacaagtatgagctagaaaaccatgacttaaaaactaagaatgaagggttaaaaactgagaatgaagggttaaaaactgagaatgaagggttaaaaactgagaatgaagggttaaaaactgagaagaaagaacatgaagcagaaaacgataagttaaaacaacagagggatacgttatctactcagaaagaaactcttgaaagagaagtacagaacacgcaatacaataatgaaacgttaaagattaagaatggtgacttaactaaagagttgaataaaactcgacaagaattagcaaataaacagcaagagagtaaagaaaatgaaaaggcccttaatgaactcttggaaaagacagtaaaagataaaattgctaaggagcaagaaaataaagaaaccattggtacccttaaaaaaatcttggatgagacagtaaaagataaaattgctaaggagcaagaaaataaagaaaccattggtacccttaaaaaaatcttggatgagacagtaaaagataaacttgcgaaagagcaaaaaagtaaacaaaacattggtgcccttaaacaagaattagctaaaaaagatgaggcaaacaaaatttcagacgcaagccgtaagggtcttcgtcgtgacttagacgcatcgcgtgaagctaagaagcaattagaagctgaacaccaaaaacttgaagaacaaaacaagatttcagaagcaagtcgcaaaggccttcgccgtgatttagacgcatcacgtgaagctaagaagcaattagaagctgaacaccaaaaacttgaagaacaaaacaagatttcagaagcaagtcgcaaaggccttcgccgtgatttagacgcatcacgtgaagctaagaaacaagttgaaaaagctttagaagaagcaaacagcaaattagctgctcttgaaaaacttaacaaagagcttgaagaaagcaagaaattaacagaaaaagaaaaagctgagctacaagcaaaacttgaagcagaagcaaaagcactcaaagaacaattagcaaaacaagctgaagaacttgcaaaactaagagctggaaaagcatcagactcacaaacccctgatacaaaaccaggaaacaaagctgttccaggtaaaggtcaagcaccacaagcaggtacaaaaccaaaccaaaacaaagcaccaatgaaggaaactaagagacagttaccatcaacaggtgaaacagctaacccattcttcacagcggcagcccttactgttatggcaacagctggagtagcagcagttgtaaaacgcaaagaagaaaattaa

SEQ ID NO: 69 - protein, 483 aa, Artificial Sequence
  Synthetic Construct
MAKNNTNRHYSLRKLKKGTASVAVALSVIGAGLVVNTNEVSARVFPRGTVENPDKARELLNKYDVENSMLQANNDKLTTENNNLTDQNKNLTTENKNLTDQNKNLTTENKNLTDQNKNLTTENKELKAEENRLTTENKGLTKKLSEAEEEAANKERENKEAIGTLKKTLDETVKDKIAKEQESKETIGTLKKTLDETVKDKIAKEQESKETIGTLKKTLDETVKDKIAKEQESKETIGTLKKILDETVKDKIAREQKSKQDIGALKQELAKKDEGNKVSEASRKGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAGKASDSQTPDAKPGNKVVPGKGQAPQAGTKPNQNKAPMKETKRQLPSTGETANPFFTAAALTVMATAGVAAVVKRKEEN

SEQ ID NO: 70 - DNA, 2111 bp, Artificial Sequence
  Synthetic Construct
gatctagaagagattgaaaaacagtatgatgtgatcgtgacagatgttatggtaggaaaaagcgatgagttagaaattttctttttctacaaaatgattccagaagcgattattgacaagctcaatgtgtttttaaacatcagctttgcagacagcttggccactagagcaaacccatccaagaaccccttggactttcatcgcaaagagcttacctttacccactccccccaacaacgttttgcacgccccccccgcacgaagttagacagcctagccgcagaaactcaaaaacagattcatcattaatagcatttaggtcaaaagtggcaaaagctaaaaaagctggtctttaccttttggcttttattatttacaatacaattattagagttaaaccctgaaaatgagggtttttcctaaaaaatgataacataaggagcataaaaatggctaaaaataacacgaatagacactattcgcttagaaaattaaaaaaaggtactgcatcagtagcagtggctttgagtgtaataggggcaggattagttgtcaatactaatgaagttagtgcaagagtgtttcctagggggacggtagaaaacccggacaaagcacgagaacttcttaacaagtatgacgtagagaactctatgttacaagctaataatgacaagttaacaactgagaataataacttaacagatcagaataaaaacttaacaactgagaataaaaacttaacagatcagaataaaaacttaacaactgagaataaaaacttaacagatcagaataaaaacttaacaactgagaataaggagttaaaagctgaggagaataggttaacaactgagaataaagggttaactaaaaagttgagtgaagctgaagaagaagcagcaaataaagagcgagaaaataaagaagccattggtacccttaaaaaaaccttggatgagacagtaaaagataaaattgctaaggagcaagaaagtaaagaaaccattggtacccttaaaaaaaccttggatgagacagtaaaagataaaattgctaaggagcaagaaagtaaagaaaccattggtacccttaaaaaaaccttggatgagacagtaaaagataaaattgctaaggagcaagaaagtaaagaaaccattggtacccttaaaaaaatcttggatgagacagtaaaagataaaattgcgagagagcaaaaaagtaaacaagacattggtgcccttaaacaagaattagctaaaaaagatgaaggaaacaaagtttcagaagcaagccgtaagggtcttcgccgtgacttggacgcatcacgtgaagctaagaaacaggttgaaaaagatttagcaaacttgactgctgaacttgataaggttaaagaagaaaaacaaatctcagacgcaagccgtcaaggtcttcgccgtgacttggacgcatcacgtgaagctaagaaacaagttgaaaaagctttagaagaagcaaacagcaaattagctgctcttgaaaaacttaacaaagagcttgaagaaagcaagaaattaacagaaaaagaaaaagctgagctacaagcaaaacttgaagcagaagcaaaagcactcaaagaacaattagcgaaacaagctgaagaacttgcaaaactaagagctggaaaagcatcagactcacaaacccctgatgcaaaaccaggaaacaaagttgttccaggtaaaggtcaagcaccacaagcaggtacaaaacctaaccaaaacaaagcaccaatgaaggaaactaagagacagttaccatcaacaggtgaaacagctaacccattcttcacagcggcagcccttactgttatggcaacagctggagtagcagcagttgtaaaacgcaaagaagaaaactaagctatcactttgtaatactgagtgaacatcaagagagaaccagtcggttctctcttttatgtatagaagaatgagattaaggaggtcacaaactaaacaactcttaaaaagctgacctttactaataatcgtcttttttttataataaagatgttaataatataattgataaatgagatacatttaatgattatgacaaaagcaagaaaa

SEQ ID NO: 71 - protein, 400 aa, Artificial Sequence
  Synthetic Construct
MVRKDANRQYSLRKLKKSTASVAVALSALGVGLAVNQTEVSAAPLTRATADNKDELIKRANGYEIQNHQLTVENKKLKIDKEQLTKENDDLKTEKDQLEQRSEKLATQKENLEKEVAEAKHKNETLNINNDDLTKKLNETRQELANKQQESKENEKTLNELLEKTVKDKIAREQKSKQDFGALKQELAKKEEQNKISEASRKGLRRDLDASREAKKQVEKDLANLTAELDKVKEEKQISDASRQGLRRDLDASREAKKQVEKALEEANSKLAALEKLNKELEESKKLTEKEKAELQAKLEAEAKALKEQLAKQAEELAKLRAEKASDSQTPDAKPGNKAVPGKGQAPQAGTKPNQNKAPMKETKRQLPSTGEAANPFFTAAAATVMVSAGMLALKRKEEN

SEQ ID NO: 72 - DNA, 1203 bp, Artificial Sequence
  Synthetic Construct
atggttagaaaagatgcaaatagacagtattcgcttagaaaattaaaaaaaagtactgcttcagtagcggttgctttgagtgccttaggggtaggattagcggttaaccaaacagaagttagcgcagcacctcttactcgagctacagcagacaataaagacgaattaataaaaagagctaacggttatgagatacagaaccatcagttaacagttgagaataaaaaattaaaaattgataaggaacagttaacaaaagagaatgatgatttaaaaactgagaaggatcagttagaacaacggagtgagaagttagctactcagaaagaaaatcttgaaaaagaagtagcggaagcgaaacacaagaatgaaacgttaaacattaataatgatgacttaactaaaaagttgaatgaaactcgacaagaattagcaaataaacagcaagagagtaaagaaaatgaaaagacccttaatgaactcttggaaaagacagtaaaagataaaattgcgagagagcaaaaaagtaaacaagactttggtgcccttaaacaagaattggctaaaaaagaagaacaaaacaaaatttcagaagcaagtcgtaaaggtcttcgtcgtgacttagatgcatcacgtgaagctaagaaacaagttgaaaaagatttagcaaacttgactgctgaacttgataaggttaaagaagaaaaacaaatctcagacgcaagccgtcaaggtcttcgccgtgacttggacgcatcacgtgaagctaagaaacaagttgaaaaagctttagaagaagcaaacagcaaattagctgctcttgaaaaacttaacaaagagcttgaagaaagcaagaaattaacagaaaaagaaaaagctgagctacaagcaaaacttgaagcagaagcaaaagcactcaaagaacaattagcaaaacaagctgaagaacttgcaaaactaagagctgaaaaagcatcagactcacaaacccctgatgcaaaaccaggaaacaaagctgttccaggtaaaggtcaagcaccacaagcaggtacaaaacctaaccaaaacaaagcaccaatgaaggaaactaagagacagttaccatcaacaggtgaagcagccaacccattctttacagcagcagctgcaacagtgatggtatctgcgggtatgcttgctctaaaacgcaaagaagaaaactaa

SEQ ID NO: 73 - protein, 162 aa, Artificial Sequence
  Synthetic Construct
MNKKMKMFIICAVFILIGACKIHTSYDEQSNGEVKVKKIEFSEFTVKIKNKNNSNNWADLGDLVVRKEKDGIETGLNAGGHSATFFSLEEEEINNFIKAMTEGGSFKTSLYYGYNDEESDKNVIKNKEIKTKIEKINDTEYITFLGDKINNSAGGDKIAEYA

SEQ ID NO: 74 - DNA, 487 bp, Artificial Sequence
  Synthetic Construct
atgaataagaaaatgaaaatgtttattatttgtgctgtttttatacttataggtgcttgcaagattcatacttcatatgatgagcaaagcaatggagaggtaaaggtcaaaaaaatagaattctctgaatttactgtaaaaattaaaaataagaataatagtaataactgggcagacttaggagatttagttgtaagaaaagaaaaagatggtattgaaacgggtttaaacgctgggggacattcggctacattcttttcattagaagaggaagaaattaataactttataaaagcaatgactgaaggtggatcatttaaaactagtttgtattatggatataatgacgaagaaagtgataaaaatgtcattaagaataaagagataaaaacaaagatagaaaaaattaatgatactgaatatattacatttttaggagataaaattaataacagtgcggggggagacaaaatagctgaatatgcaa

SEQ ID NO: 75 - protein, 179 aa, Artificial Sequence
  Synthetic Construct
MNKKMKMFIICFIFALISSCKNHTLYDGQSNGEAKVKKIEFSEFTVKIKNKNNSNNWADLGDLVVRKEEDGIETGLNVGKGDSDTFAGYTATFFSLEESEVNNFIKAMTEGGSFKTSLYYGYKDEQSNANGIQNKEIITKIEKIDDFEYITFLGDKIKDSGDKVVEYAILLEDLKKNLK

SEQ ID NO: 76 - DNA, 540 bp, Artificial Sequence
  Synthetic Construct
atgaataagaaaatgaaaatgtttattatttgctttatttttgctttgataagttcttgtaaaaatcatactttatatgatgggcaaagtaatggagaggcaaaggttaaaaaaatagaattctctgaatttactgtaaaaattaaaaataagaataatagtaataactgggcagacttaggagatttagttgtaagaaaagaagaagatggtattgaaacggggttaaatgttgggaagggagactctgatacattcgcaggatacaccgctacattcttttcattagaagagtcagaagttaataactttataaaagcaatgactgaaggtggatcatttaaaactagtttatattatggatataaggacgaacaaagtaatgcaaatggtatccaaaataaggagataataacaaaaatagaaaaaattgatgattttgaatatattacatttttaggagataaaattaaggattcaggagataaagttgttgaatatgcaatactactagaagatcttaaaaaaaatttaaaatag

SEQ ID NO: 77 - protein, 186 aa, Artificial Sequence
  Synthetic Construct
MNKKMKMFIVCAVFILIGACKIHTSYDEQSSGEINHTLYDEQSNGELKLKKIEFSKFTVKIKNKDNNSNWTDLGDLVVRKEENGIDTGLNAGGHSATFFSLKESEVNNFIKAMTKGGSFKTSLYYGYKYEQSSANGIQNKEIITKIESINGAEHIAFLGDKINNGVGGDKTAEYAIPLEVLKKNLK

SEQ ID NO: 78 - DNA, 561 bp, Artificial Sequence
  Synthetic Construct
atgaataagaaaatgaaaatgtttattgtttgtgctgtttttatacttataggtgcttgcaaaattcatacttcatatgatgagcaaagtagtggtgagataaaccatactttatatgatgagcaaagtaatggtgagttaaaacttaaaaaaatagaattctctaaatttactgtaaaaattaaaaataaagataataatagtaactggacagacctaggagatttagttgtaagaaaagaagaaaatggtattgatacgggtttaaacgctgggggacattcggctacattcttttcattaaaagaatcagaagttaataactttataaaagcaatgactaaaggcggatcatttaaaactagtttgtattatggatataagtacgaacaaagtagtgcaaatggtatccaaaacaaagagatcataacaaaaatagaaagtattaatggtgctgaacatattgcgtttttaggagataaaattaataacggtgtggggggagataaaacagctgaatatgcaataccactagaagtgcttaaaaaaaatttaaaatag

SEQ ID NO: 79 - protein, 698 aa, Artificial Sequence
  Synthetic Construct
MVSSYMFARGEKMNNKMFLNKEAGFLVHTKRKRRFAVTLVGVFFLLLACAGAIGFGQVAYAADEKTVPNFKSPDPDYPWYGYDSYRGIFARYHNLKVNLKGSKEYQAYCFNLTKYFPRPTYSTTNNFYKKIDGSGSAFKSYAANPRVLDENLDKLEKNILNVIYNGYKSNANGFMNGIEDLNAILVTQNAIWYYSDSAPLNDVNKMWEREVRNGEISESQVTLMREALKKLIDPNLEATAANKIPSGYRLNIFKSENEDYQNLLSAEYVPDDPPKPGDTSEHNPKTPELDGTPIPEDPKRPDESSEPALPPLMPELDGEEVPEVPSESLEPALPPLMPELDGEEVPEVPSESLEPALPPLMPELDGEEVPEVPSESLEPALPPLMPELDGEEVPEVPSESLEPALPPLMPELDGEEVPEKPSVDLPIEVPRYEFNNKDQSPLAGESGETEYITEVYGNQQNPVDIDKKLPNETGFSGNMVETEDTKEPEVLMGGQSESVEFTKDTQTGMSGQTTPQVETEDTKEPEVLMGGQSESVEFTKDTQTGMSGQTTPQVETEDTKEPGVLMGGQSESVEFTKDTQTGMSGQTTPQVETEDTKEPGVLMGGQSESVEFTKDTQTGMSGFSETVTIVEDTRPKLVFHFDNNEPKVEENREKPTKNITPILPATGDIENVLAFLGILILSVLSIFSLLKNKQNNKV

SEQ ID NO: 80 - DNA, 2097 bp, Artificial Sequence
  Synthetic Construct
atggtaagctcatatatgtttgcgagaggagagaaaatgaataacaaaatgtttttgaacaaagaagccggttttttggtacacacaaaaagaaaaaggcgatttgctgtcactttagtgggagtcttttttctgcttttggcatgtgcgggtgctatcggttttggtcaagtagcctatgctgcggatgagaagactgtgccgaattttaaaagcccagatccagattatccctggtatggttatgattcgtatagaggaatatttgcaagatatcacaatttaaaagtaaatctaaaaggaagtaaggagtatcaagcgtattgttttaacctaacaaaatactttcctcgccccacttatagtactacaaataatttttacaagaaaattgatgggagtggatcagcgttcaaatcttatgcagcgaatcctagggttttagatgagaatttagataaattagaaaaaaatatactgaatgtaatttataatggatataaaagtaatgcaaatggttttatgaatggtatagaagatcttaatgctatactagtaactcaaaacgctatttggtactattcagatagtgctccattaaatgatgttaataaaatgtgggaaagagaggttcggaatggggagattagtgagtcacaagttactttaatgcgtgaggcattgaaaaaactaattgatcccaatttagaagctactgcagctaataaaatcccatcaggatatcgtttaaatatctttaagtctgaaaatgaagattaccaaaatcttttaagtgctgaatatgtacctgatgatccccctaaacctggtgatacgtcagaacataatcctaaaactcccgagttggatggcactccaattcccgaggacccaaaacgtccagatgagagttcagaacctgcgcttcccccattaatgccagagctagatggtgaagaagtcccagaagttccaagcgagagcttagaacctgcgcttcccccattgatgccagagctagatggtgaagaagtcccagaagttccaagcgagagcttagaacctgcgcttcccccattgatgccagagctagatggtgaagaagtcccagaagttccaagcgagagcttagaacctgcgcttcccccattaatgccagagctagatggtgaagaagtcccagaagttccaagcgagagcttagaacctgcgcttcccccattgatgccagagttagatggtgaagaagtccctgaaaaacctagtgttgacttacctattgaagttcctcgttatgagtttaacaataaagaccagtcacctctagcgggtgagtctggtgagacggagtatattaccgaagtctatggaaatcaacagaaccctgttgatattgataaaaaacttccgaatgaaacaggtttttcaggaaatatggttgagacagaagatacgaaagagccagaagtgttgatgggaggtcaaagtgagtctgttgaatttactaaagacactcaaacaggcatgagtggtcaaacaactcctcaggttgagacagaagatacgaaagagccagaagtgttgatgggaggtcaaagtgagtctgttgaatttactaaagacactcaaacaggcatgagtggtcaaacaactcctcaggttgagacagaagatacgaaagagccaggagtgttgatgggaggccaaagtgagtctgttgaatttactaaagacactcaaacaggcatgagtggtcaaacaactcctcaggttgagacagaagacacgaaagagccaggagtgttgatgggaggtcaaagtgagtctgttgaatttactaaagacactcaaacaggcatgagcggtttcagtgaaacagtgaccattgttgaagatacgcgtccgaagttagtgttccattttgacaataatgagcccaaagtggaagagaatcgggaaaagcctacaaaaaatataacacctatccttcctgcaacaggagatattgagaatgttttggcctttcttggaatccttattttgtcagtactttctatttttagccttttaaaaaacaaacaaaacaataaagtctga

SEQ ID NO: 81 - protein, 244 aa, Artificial Sequence
  Synthetic Construct
MARWRRRLGVAALGAAMLASLAPAARASLVITGTRVIYNAGSPETTVKMSNEGQAPALMQAWIDDGNAEAKPDEVQVPFFLTPPLARVDPGKGQTLRIFFNGYPDGKTLPSDRESVFWLNVLEVPPKATPEEGHGVLQLTIRSRLKLFYRPKGLSGNPLTAAADLTFKRKPNGVLEVHNPTPYYVNLQKLEVGENGAHGSKTPWMLAPLSSDELRLKGTGAKSVQYWAIDDFGGVTPYQAAIAD

SEQ ID NO: 82 - protein, 873 aa, Artificial Sequence
  Synthetic Construct
MKQIPLILAMSLAFAAAAKGESAPDMQAAVNFDSAMLWGGANGADLSRFNYSNALRPGNYIVDIYANNYPLIRQQVRFVAAQTSGQGLKTAPAVACFTYGQLEAMQVRLRALDPALVADLKSSGRCEVLGKLFPDSRESFDFGENRLEVSIPQAYTINRFRRDISPDEWDSGITAFRLGYQYNYADYIGGLRAGRRLDLNLYSGFNFKGWYLRNSSTLGWGQGRFTRRSQRTSLQTDIPSWRARLVFGDVFSSGEYFAPYSMRGMLVGSDTAMLPYSERLYRPTIRGVARTRANVKVYQAGVLVFQDAVPPGPFAIDDYSPASYGGDLRVVVTEANGAVQTFTVPYASAVRLILPGQTQWSFSAGRYRNYRNDGQDRPWVTQLTGRHGVADGVNLYGGLLIAQAYQAGLAGLSWNTPWGAMAADATLSRSQLSTTGNANGSSLRFSYSKTLSGTNTAIRLATLRYSSSGFWNFADAVNAGPVETNGRNGRFGLYSLLGRERPRGDFSVTLSQPLGGYGSLYVSALRRTYWGSSRVDQQTQLGYSTQVGRVGVNLDVSRTENRRSTEHQVMLNLSIPLYGATSSGVVTGSLARTGSAPVQQSVNYSGMSGERDQYTYGLGVQRAGTSAQYALNGSWSGTYGEVSGQLTHGRSYSQYQINGSGGLVAHAGGVTFGQYQAGTIGLIQAEAAAGAKVVNTRNAAVDRSGYGLVSLTPYSLNEVELSPQDLPLDVQLESTVEQVIPRAAAVVALRFPTRHDVAAMLVAEPGSEGALVFGTEVRDGAGKVVGVAGQGASALVRGVSASGTLEVTRADGSICRATYDLKSAGQAVHGLPRIALACAPQGGGERGARAAGQAVAQPSAISISGKDHEPDIR

SEQ ID NO: 83 - protein, 376 aa, Artificial Sequence
  Synthetic Construct
MSQIFADRRAAVPARVISFCGAALAVWAGLAVQPAMAVDPPVDCGRALGLHFWSSASLISDQTPDGTLIGKPVVGRSLLSKSCKVPDDIKEDLSDNHDGEPVDIVLELGSNYKIRPQSYGHPGIVVDLPFGSTEETGIAIYIDFGSSPMQKVGERQWLYPQKGEVLFDVLTINGDNAEVRYQAIKVGPLKRPRKLVLSQFPNLFTYKWVFMRGTSQERVLAQGTIDTDVATSTIDLKTCRYTSQTVSLPIIQRSALTGVGTTLGMTDFQMPFWCYGWPKVSVYMSATKTQTGVDGVALPATGQAAGMASGVGVQLINGKTQQPVKLGLQGKIALPEAQQTESATFSLPMKAQYYQTSTSTSAGKLSVTYAVTLNYD

SEQ ID NO: 84 - DNA, 5441 bp, Artificial Sequence
  Synthetic Construct
tcgcggcctgccgcggctcggcgcatggggattcgcagggttctcatgcgccggccaatgccggatagcggtgcaattgccgaccatttcgcgcaccgcgctcaaggacgtagggtcgacggcaggcgggacagtttttgacgtgaaactgaccgagtgtccgcaggcattgaatggtcagcaagtgggattgttcttcgaatctggtggcacggttgactatacgtcgggaaacctgtttgcgtatcgggccgatagtcagggcgtcgaacaggtaccgcagacgaaagccgacaacgtgcaagccaatctggatggttccgctattcatttgggccgcaacaagggtgcgcaggctgctcagacgtttctggtatcgcagacggctgggtcgtcgacgtacggggcgaccctgcgctatctggcatgctacatccgttcgggcgctggttccattgttgcggggaatctccgcagtcaggtggggttctccgtgatgtatccgtagcccgtgaaagaggggtcacccactgcggggggccccggtacgggatggtcggcttgtcacgagattcttgttttccatttctttcttttcactcggtcgcagcgccggcttgatgcatgcaaagcatcgatagctacgaacggccgcgattcttgaatcatgaatacatacgcttgtgacggggcgctcgcgcagagccggccccagggatggtttacgcctgcatttacggtaaagcggcaaggcggcatggcgcgctggcgccggcggctgggcgtcgcggcgctgggcgccgccatgctggcgagcctggcgccggccgcccgggccagcctcgtcatcacgggtacgcgcgtgatctacaacgcgggctcgcccgagaccacggtgaagatgagcaacgaggggcaggcccccgcgctgatgcaggcatggatcgatgacggcaatgccgaggcgaagccggacgaagtccaggtgccgttcttcctgacgcctccgctggcgcgggtggacccgggcaaggggcagaccttgcgcattttcttcaatggctacccggatggcaagacgctgccgtccgaccgcgaatcggtcttctggctgaacgttctcgaagtgccgcccaaggcgacgcccgaggaaggccacggggtcctgcagctgaccatccgctcgcgcctgaagctgttctatcggcccaagggcctgtccggcaacccgctgacggccgccgccgacctgaccttcaagcgcaagcccaacggcgtgctggaggtgcacaacccgacgccgtactacgtgaacctgcaaaaactggaggtgggcgagaacggcgcgcacggcagcaagacgccgtggatgctggcgccgctttcctcggacgagctgcgcctgaaagggacgggggccaagtcggtgcagtactgggccatcgacgacttcggcggcgtgacgccatatcaggcggccatagcggactgacaagacgggggcaccgggactggcgcgcgtcatggggacgttgccgaaaccgacgctgaatcatgaaacagataccactgattctcgcgatgagccttgccttcgccgccgcggcgaagggcgaatccgcgccggatatgcaggcggcggtcaacttcgactcggccatgctgtggggcggcgccaatggcgcggatctgagccgattcaactacagcaacgcgctgcggccgggcaactacatcgtcgacatttacgccaacaactatccgctgatccggcagcaggttcgattcgtcgcggcgcagacgtccgggcagggcttgaagacggcgccggcggtggcgtgtttcacctatggccagctggaagcgatgcaggtccggctgcgcgcgctcgatccggccttggtcgccgacctcaaatccagcgggcgctgcgaggtgctgggcaagctgttcccggatagccgcgagtcgttcgatttcggcgagaaccgcctggaagtcagcattccgcaagcctatacgatcaaccgcttccggcgcgacatctcgcccgatgagtgggatagcggcattactgcgttccgcctgggataccagtacaactatgccgattacatcggcggcttgcgcgcggggcggcggctggacctgaacctctacagcgggttcaatttcaagggctggtacctgcgcaacagctcgacgctgggatggggccagggccgcttcacgcgccgcagccagcgcaccagcctgcagacggatatcccgtcctggcgggcgcgcctggtgttcggcgacgtgttctccagcggcgagtacttcgcgccgtactcgatgcgcggcatgctggtcggcagcgataccgccatgctgccgtactcggagcgcctgtaccggccgaccatacgcggcgttgcgcgcactcgggccaacgtcaaggtgtatcaggccggcgtgctggttttccaggacgccgtgcctcccgggccgttcgcgatcgacgattacagtccggcctcgtatggcggcgacctgcgggtggtggtgaccgaggcgaatggcgcggtccagacgttcaccgtgccgtacgccagcgcggtgcgcctgatcctgccgggccagacgcagtggtcgttcagcgccgggcgctatcgcaactatcgcaacgatgggcaggaccgtccctgggtgacccagctgacgggccgccatggcgtggccgacggcgtcaacctgtacggcggcctgctgatcgcccaggcgtaccaggccggcctggccggcctgagctggaacacgccctggggggcaatggcggcggacgccacgctgtcgcgcagccagctgtccaccaccggcaatgccaacgggtcgagcctgcggttctcctacagcaagaccttgtccggcaccaacacggcgatccggctggctacgctgcgctactcgtcgagcgggttctggaatttcgccgatgcggtcaatgccggcccggtcgagacgaacgggcgcaacgggcggttcggactctattcgctgctgggccgcgagcggccgcgcggcgatttcagcgtcacgctgagccaaccgctgggtggatacggcagcctgtacgtgtcggcgctgcggcggacctactggggttcgtcgcgggtcgaccagcagacccagctgggctattcgacccaggtcggccgcgtaggcgtgaacctggatgtctcgcgtacggagaatcgtcgcagcaccgagcatcaggtcatgctgaatctgtccattccgctttatggcgcgacctccagcggcgtggtgacgggatcgctggcgcgtaccggctcggcgcccgtacagcaaagcgtcaactacagcggcatgtcgggcgagcgcgatcagtacacctatggcctgggcgtgcagcgcgccggcacgagcgcgcagtacgcgttgaacggctcctggtcaggcacgtacggggaggtaagcggacagctcacccatgggcgcagctacagccagtaccagatcaacggcagcggcggcttggtggcgcacgcgggcggcgtgaccttcggccagtaccaggcgggcaccatcggcctgatccaggccgaggcggcggcaggcgcaaaggtcgtcaatacgcgcaacgccgccgtggaccgcagcggctatgggctggtgtcgctgacgccgtattccctcaacgaggtcgaactgtctccgcaggacctgcccctagacgttcagctggagtccacggtggagcaagtcatcccgcgggcggccgccgtcgtggcgctgcgctttccgacgcgccatgacgtcgccgccatgctggttgccgaacccggcagcgaaggcgcgctggtattcggcaccgaagtgcgcgacggcgccggcaaggtggtgggcgtggccggccagggcgcaagcgcgctggtcagaggcgtgtcggcatcgggcacgctggaggtgacgcgcgcagacggttcgatatgccgcgccacgtacgacttgaaatcggccgggcaagccgtgcatggcctgccgcgcattgcgctggcgtgcgcgccgcaaggcggcggcgaacgtggtgcgcgggcggcgggccaggccgtggcgcagccatcagctatctccatttcgggtaaagatcatgagccagatattcgctgaccgccgggccgccgtgcccgcgcgcgtaatttccttctgcggggccgcgcttgccgtctgggcaggcctggccgtgcagcccgccatggccgtcgatccgccggtggactgcggccgggcgctaggcttgcatttctggtcgagcgcctcgctcatctccgaccagacacccgatgggacgctgatcggcaagcccgtggtcgggcggtccctgctgtccaagagctgcaaggtgccggacgacatcaaggaagacctcagcgacaaccatgacggcgaaccggtcgacatcgtgctggaactgggcagtaactacaagatccggccgcagtcctatggccatccgggcatcgtggtcgacttgccgttcggctccacggaggagaccggcatcgccatctatatcgatttcggcagttcgccgatgcagaaggtcggcgaacggcagtggctgtatccccagaaaggcgaagtgcttttcgacgtgctcaccatcaacggcgacaacgcggaggttcgctatcaggcgatcaaggtcgggccactcaagcggccgcgcaagctggtgctgtcgcagtttccgaacctgttcacctacaagtgggttttcatgcgcgggaccagccaggagcgcgtgctggcgcaggggaccatcgacaccgacgtcgccaccagcaccatcgacctgaaaacctgccgctatacctcgcagacggtcagcctgcccatcatccagcgttccgcgttgaccggcgtcggtacgaccctggggatgaccgatttccagatgccgttctggtgctatggctggccaaaggtatcggtgtacatgagcgcgacgaagacgcagaccggcgtagacggcgtggcgttgccggcgaccggccaggcggccggcatggccagcggcgtaggcgtccagttgatcaacggcaagacgcagcagccggtcaagctgggcctgcagggcaagatcgccttgcccgaggcgcagcagactgagtcggcgacgttctcgctgcccatgaaggcgcagtactaccagacctccacttcaacctcggcgggcaagctgtccgtcacctacgccgtgaccttgaactatgactgacgcaacgaaccgtttccggccgggcctggttggtcgggcgctggtccgggcaggcctgctgttcgccgtggccgcctgtgcgcaggcgcagctgctgccgggcgcgcgcgacctcaaccgtatcgacgatcgccagcgcaaggagcagctgcag

SEQ ID NO: 85 - protein, 192 aa, Artificial Sequence
  Synthetic Construct
MQLTKKYLLVIFLLLSLASCDLFNKNKKLDADLLKTLDNLLKTLDNNQKQALIYFKDKLQDKKYLNDLMEQQKSFLDNLQKKKEDPDLQDRLKKTLNSEYDESQFNKLLNELGNAKAKQFLQQLHIMLQSIKDGTLTSFSSSNFNDLQNLEQKKERALQYINGKLYVEYYFYINGISNADNFFETIMEYLKT

SEQ ID NO: 86 - DNA, 579 bp, Artificial Sequence
  Synthetic Construct
atgcaattaacaaaaaaatatttactagtaatttttttattattaagccttgctagctgtgatttattcaataaaaacaaaaaattagatgctgatttactaaaaactcttgataatttactaaaaactcttgataataatcaaaaacaagctctcatttactttaaagataaacttcaagataaaaaatatttaaatgatttaatggaacaacagaaaagttttttagacaatttacagaaaaagaaagaagatcctgatttgcaagatagacttaaaaaaacactaaactctgagtatgatgagagtcaattcaataaattacttaatgaacttggaaatgctaaggctaaacaattccttcaacaattgcatataatgctacagtctattaaagacggcacgcttacaagcttttcatcttcaaatttcaatgacttgcaaaatttagagcaaaaaaaagaacgggcattgcaatatatcaatggtaaattatatgttgaatactatttctatatcaatggaattagcaatgcagacaatttttttgaaactataatggaatatttaaaaacttaa

SEQ ID NO: 87 - protein, 247 aa, Artificial Sequence
  Synthetic Construct
MPKLVLVRHGQSEWNEKNLFTGWVDVKLSAKGQQEAARAGELLKEKKVYPDVLYTSKLSRAIQTANIALEKADRLWIPVNRSWRLNERHYGDLQGKDKAETLKKFGEEKFNTYRRSFDVPPPPIDASSPFSQKGDERYKYVDPNVLPETESLALVIDRLLPYWQDVIAKDLLSGKTVMIAAHGNSLRGLVKHLEGISDADIAKLNIPTGIPLVFELDENLKPSKPSYYLDPEAAAAGAAAVANQGKK

SEQ ID NO: 88 - DNA, 744 bp, Artificial Sequence
  Synthetic Construct
atgccaaagttagttttagttagacacggtcaatccgaatggaacgaaaagaacttattcaccggttgggttgatgttaaattgtctgccaagggtcaacaagaagccgctagagccggtgaattgttgaaggaaaagaaggtctacccagacgtcttgtacacttccaagttgtccagagctatccaaactgctaacattgctttggaaaaggctgacagattatggattccagtcaacagatcctggagattgaacgaaagacattacggtgacttacaaggtaaggacaaggctgaaactttgaagaagttcggtgaagaaaaattcaacacctacagaagatccttcgatgttccacctcccccaatcgacgcttcttctccattctctcaaaagggtgatgaaagatacaagtacgttgacccaaatgtcttgccagaaactgaatctttggctttggtcattgacagattgttgccatactggcaagatgtcattgccaaggacttgttgagtggtaagaccgtcatgatcgccgctcacggtaactccttgagaggtttggttaagcacttggaaggtatctctgatgctgacattgctaagttgaacatcccaactggtattccattggtcttcgaattggacgaaaacttgaagccatctaagccatcttactacttggacccagaagctgccgctgctggtgccgctgctgttgccaaccaaggtaagaaataa

SEQ ID NO: 89 - protein, 612 aa, Artificial Sequence
  Synthetic Construct
MFASKNERKVHYSIRKFSIGVASVAVASLFMGSVVHATEKEVTTQVATSSNKANKSQTEHMKAAKQVDEYIEKMLSEIQLDRRKHTQNVGLLTKLGAIKTEYLRGLSVSKEKSTAELPSEIKEKLTAAFEQFKKDTLKSGKKVAEAQKKAKDQKEAKQEIEALIVKHKGREIDLDRKKAKAAVTEHLKKLLNDIEKNLKKEQHTHTVELIKNLKDIEKTYLHKLDESTQKAQLQKLIAESQSKLDEAFSKFKNGLSSSSNSGSSTKPETPQPETPKPEVKPELETPKPEVKPEPETPKPEVKPEPETPKPEVKPELETPKPEVKPEPETPKPEVKPEPETPKPEVKPEPETPKPEVKPELETPKPEVKPELETPKPEVKPEPETPKPEVKPELETPKPEVKPEPETPKPEVKPELETPKPEVKPEPETPKPEVKPELETPKPEVKPEPETPKPEVKPEPETPKPEVKPEPETPKPEVKPELETPKPEVKPELETPKPEVKPEPETPKPEVKPELETPKPEVKPELEIPKPEVKPDNSKPQADDKKPSTPNNLSKDKQSSNQASTNENKKQGPATNKPKKSLPSTGSISNLALEIAGLLTLAGATILAKKRMK

SEQ ID NO: 90 - DNA, 2793 bp, Artificial Sequence
  Synthetic Construct
aatgagaaacgaatccttagcaatggcgggaaagaatttggagttgagaatacaaaacgattaactatggctcatattgttttttatctctcttgcttggttgaggcaatggtgcacaagacaatttttgatggcatgggcatggttggtttagtcttgcttattttttctatgctgatgttgatgttggtgattcacttgttgggagatatttggacagtgaagcttatgcttgtcaataatcacaaatatgtagatcatatcttgtttaggacagtaaaacaccctaattactttttaaatattttacctgagttgattggcttgaccttgttgagtcatgcctatatgacttttgttttagtttttccagtttatgcagttattttgtatcgacgaatagctgaagaggaaaagctattacatgaagttataatcccaaatggaagcataaagagataaatacaaaattcgatttatatacagttcatattgaagtgatatagtaaggttaaagaaaaaatatagaaggaaataaacatgtttgcatcaaaaaacgaaagaaaagtacattattcaattcgtaaatttagtattggagtagctagtgtagctgttgccagtctttttatgggaagtgtggttcatgcgacagagaaggaggtaactacccaagtagccacttcttctaataaggcaaataaaagtcagacagaacatatgaaagctgctaaacaagtcgatgaatatatagaaaaaatgttgagtgagatccaattagatagaagaaaacatacccaaaatgtcggcttactcacaaagttgggcgcaattaaaacggagtatttgcgtggattaagtgtttcaaaagagaagtcgacagctgagttgccgtcagaaataaaagaaaagttaaccgcagcttttgagcagtttaaaaaagatacattgaaatcaggaaaaaaggtagcagaagctcagaaaaaagccaaggatcaaaaagaagctaaacaggagatagaagctctaatcgttaaacataaggggcgagaaatcgatttagatcgaaagaaggcaaaggctgcagttactgaacatctaaaaaaattattgaatgacatcgagaaaaatttaaaaaaagagcaacatacccatactgtagagttaattaaaaacttgaaagatattgaaaaaacgtatttgcataagttagatgaatcaacgcaaaaagcccaactacagaaactgatcgcagaaagtcaatcaaaactagatgaagctttttctaaatttaaaaatggcttatcttcttcgtcgaattcaggctcctccactaaaccagaaactccgcagccggaaacaccaaaaccagaggttaaaccagagctggaaacaccaaaaccagaggttaaaccagagccggaaacaccaaaaccagaggttaaaccagagccggaaacaccaaaaccagaggttaaaccagagctggaaacaccaaaaccagaggttaaaccagagccggaaacaccaaaaccagaggttaaaccagagccggaaacaccaaaaccagaggttaaaccagagccggaaacaccaaaaccagaggttaaaccagagctggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagctggaaacaccaaaaccagaggttaaaccagagccggaaacaccaaaaccagaggttaaaccagagccggaaacaccaaaaccagaggttaaaccagagccggaaacaccaaaaccagaggttaaaccagagctggaaacaccaaaaccagaggttaaaccagagctggaaacaccaaaaccagaggttaaaccagagccggaaacaccaaaaccagaggttaaaccagagctggaaacaccaaaaccagaggttaaaccagagctggaaataccaaaaccagaggttaaaccagataatagcaagccacaagcagatgataagaagccatcaactccaaataatttaagcaaggacaagcaatcttctaaccaagcttcaacaaacgaaaacaagaagcaaggtccagcaacaaataaaccgaagaagtcattgccatcaactggatctatttcaaatctagcacttgaaattgcaggtcttcttaccttggcgggggcaaccattcttgctaagaaaagaatgaaatagtgattgattcattcaaaaatgtttgtaaaaagtattcggcttagaaagaaaagagtctgggataaaaagattttgattttaggaattctttattataaatttttaaaatcgatagattagacaaaaaagcgaacaagacagaattctgagtgtcagataactgattttgttcgctttttatatttaatattatacttttgtcctaggctcttttttgcattatactttttctaattgcaagagggcttcaatctctgctagggtgctagcttgcgaaatggctccacggagtttggcagcgccagatgttccacggagatagtgaggagcgaggccgcggaattcacgaactgcgacgttttctcctttgaggttaatcaatcgtttcaagtgttcgtaggcgatcttcatctt

SEQ ID NO: 91 - protein, 240 aa, Artificial Sequence
  Synthetic Construct
MNLKQGNKILKTIFLTLLIGSVVCALYSCGDKKEEDNSELLLFLLNSLGSSNNTSTPVVTSCKDASFCRTFIATNNGAGYNGNLGGISGADAKCAAAKSSSLTGAYKALIVDGPTGRQVISALDGSVDKKDWVLYSNKQYRRSDGTTITFTTNANSIVIDNLQNGIDSGAQKFFWNGLGNNVGFLWEPLSNCNNWNSADGLITGQAGNTTELQADITPEGAFTVDNHACNTNLNLLCVEQ

SEQ ID NO: 92 - DNA, 3335 bp, Artificial Sequence
  Synthetic Construct
aattaatgtttttttaatattcataagtgtacaatggtgggctcagtattattctgatggctcggggtatttggctcaaagaatacacaccgcaaaaaatccaaaagaagcagaaaagggatctctttggtttaacatagccaattttatggttcgcacctggccttggatattaactgcacttgtaacgttagttgtgtttcctttgcacgatccgacaaaatatttttcagaaggatggatcgtaggcggcgatcgtgaaatgggatatccgattttaatgaaattgatattacccaatggaattttgggaattgtatttgcgagtttgatggccgcttttatgtctacggctgacactcatatcaattggggtgcgagttatcttgtaaacgatttttatttgagatttgtacatccaaaagcggacgataaaactttggtgaaagcgagtagaattgcagtagtcactatgtctatcatcgccattttggtagccactcagattcaatctatcgcgaacgcttggaaatttttgttggcatttgcttccggaatgggattacctcaaatactaagatggatttggtggaggactaacgcttggactgaactttccggaatgatcaccgcgttgatcctttctatgattttgtatccttcttatccaaatgttcgatcggaatatcttttattttgggttgcgataggttctgttgcggtttcgatcctagttacgtttttaaccccgccggttccacaaaacacgttagacgattttataaaaagagttgatcctatcggattttggaaaggagaagataataaaaaaagactcgaggatttttataaaaaaatctttctttggcttttaggaacggtcgctttattttttggaatgttttctttagggtattttttcttattacagttttggcaagggttcttttgtttgtttggttttgtgtttttagggatcttgtattggaaaaaagaatttggtagaaattgataaattatagtaaaataaaatattataataaactaataataaatatttatcgtaaaatgtgtataaacggtgcctaggtatacattttgttcttgaataaattatttttcgaatcaaagacttttgtagcattagtcggtaatagagttttagcgaacttatattaacgtaagaaaatcctaggtcaattttttcgtaaaaaaatagatgtgggaactactatgaataccaaaagaatcatcgtccaatccgatttttgcagccatctttaaaatttaaatcccagtttaacatgagttcgactgagaaaaaatttttctaaaagtataaattcctataattttagaatttgttcgtaaaatcgtgatttgtaatagttcccacattttacaggcaaacctaaattttgtgtgagctcgcatgcctgaatatgacatatttaattattataaatttctattttaaaagttgtaatagttcccacatttaaaaaatcaatctatgaagttctgattccaacttttttcagcatcatgggttttttatgtcgaattcacgttaaatagtctttgaaaatgtggaaactcttgcaatttttaagagtgaaaagcttttaaaaagtcagaaactgcgtaaagagacttactactcgggtgcatgaagataacactaaaattaacgcataattccagcaagaatacaaaggttctcaaaaactaggcctaacccagattttaagcttttttgtggtagatcccacattctaacttttaaaacaagttcacgatagtgttgttctgatatgtccgagtaagtaatctgtgggaactaccatattgggtttatagagagatttaaagttgatttcttttaaagctttttggatggagacttgcagagaattcctataaaacccaacttaagaaacaaatgtctatataaaacagtcattttaacaaccaaaggaaaattataaattcattatatataaatttctaataaatgatttaatgaagtttgtttataaatcaataacgtatcgtttttaaaattcataagaacctatttcaaaggtaactcaatggcactgctctgcggatttttggacgcgctgtaaatgggaaactttttataaaacaaggttttactttgataagtcaaaaaaagttttatttccagatgtagtctgcatcttataaaacaaatctgttcaagatataatataatgtctttgttgtaaactttactgatagaacttaaaaaagttttttccaatagatcataaatttatttccaaaaggtgagaaatactcacctggatctgttatttagttctaagttcgtttgattttctttttaggttaaaagtttttaaatctaaactcgaaaggataaatgcaaacgaatgaatttaaaacaaggaaataaaattttgaaaacaatatttctaacattgcttatcggatccgttgtatgcgctctttattcgtgtggggataaaaaagaagaagataattcagaactacttcttttccttctcaattcattaggttcttccaataatacttctactcccgtcgttacttcttgtaaggatgcttctttttgtagaacttttatcgctaccaataacggggcgggttataatggaaacttaggtggaatttctggagcagacgcaaagtgtgccgctgcaaaatcttctagtttaacgggagcttataaggctttgattgtcgacggtccaacgggaagacaagttatttctgctttagatggatcagtagataaaaaagattgggtcctttattcaaacaaacaataccgtagaagcgacggcactacaattacctttactacaaacgcaaattctattgttatagataatcttcaaaatggaattgattcaggagctcaaaaattcttttggaacggtttaggaaacaacgttggatttctttgggagccattatcaaactgtaataattggaattctgcggacggacttattacgggacaggctggaaatacaaccgaacttcaagcagatataactccagaaggagcttttaccgttgataaccatgcttgtaataccaatctaaatcttctctgtgtagaacagtaagtttttcaaaaacatcgcccttgaaagggcgatgtttgatttctaaagcttatataaattgcagaacttacttttgagataaaagttccatttctttagaaagacaaagtagagcggtcaaaatcaaaatgggagaaacgttaggatctttggaagattgtattgatgcaattatggattctaaaagagttgaaa

SEQ ID NO: 93 - protein, 295 aa, Artificial Sequence
  Synthetic Construct
MTYQLFKHHLVALMVTGAISVNALAKDSFLENPSANLPQQVFKNRVDIFNNETNINENKKDIAINKANIASIEKDVMRNTGGIDRLAKQELVNRARITKNELDIRKNTKSIAENTASIARIDGNLEGVNRVLQNVDVRSTENAARSRANEQKIAENKKAIENKADKADVEKNRADIAANSRAIATFRSSSQNIAALTTKVDRNTARIDRLDSRVNELDKEVKNGLASQAALSGLFQPYNVGSLNLSAAVGGYKSKTALAVGSGYRFNQNVAAKAGVAVSTNGGSATYNVGLNFEW

SEQ ID NO: 94 - DNA, 1967 bp, Artificial Sequence
  Synthetic Construct
gttcgggcgggtggtgtccgtgtacatgccgggacgcagctggacaggttcgaggtcttttaaaacggtaatttcgttggcggaataattcgttgtcataatgggttaattcataaaatcgggaagcaaaattgcgcgggattatagcaaaaaacaacggaatttttaaccgcactttaatggatttggcaaaaagtgcggtcgttttggcaggtgttttatttttttgagtaatggctcgtgtggtgatagctttaaaaaaggtctcggcaggtaactattttaaccgattaaaatataatttttttagattataagtttggagggaaagatttaaccttgtttaaagaaggattaataaatggttttttaatatgcaagattaagtattgtttctattttagggtaaaacaatagtcgtaacctattgaattgatacatttatattagaagggattcatatttttttgtggtataaattcaagccgaaaaggaggcatgttaaatcagactcttttttaattttattatggaagaatggtttttcgatttcgaaaaattagactaaaaattaaataactttattttttaaggatgaattatgacatatcaattatttaaacaccacctcgttgctttaatggtcactggtgcaatttctgtcaatgcattggctaaagactcttttctggaaaatccttctgctaatcttccacaacaagtctttaaaaacagggtggatatttttaataatgagacgaacattaatgaaaataagaaagatattgctattaataaagcaaatattgctagtatagaaaaagatgttatgcgtaacactggggggatcgatagattagctaagcaagagcttgttaacagggcaaggattactaaaaatgagttagatattcggaaaaatactaaatcaattgcagagaatacagcttctatagcccgtattgatggaaatctggaaggcgttaatcgggtgcttcaaaatgtagatgtgagatctaccgaaaatgcggcaagatcccgtgctaacgagcaaaaaatcgccgagaataaaaaagcaattgaaaataaagcggataaggctgatgtagagaaaaacagagccgatattgcagcaaattccagagcgattgcaacctttagatcttcaagccaaaacatcgcggcattaacgaccaaagttgatcgtaatactgcgcgtattgatcgattagatagccgagtcaatgaattagacaaagaagtaaaaaacggtttggcttcccaagcggcactaagcggcttattccaaccgtataatgtcggcagccttaacttgagtgcagctgttggtggttataaatctaaaacagcactagcggttggttcaggttatcgtttcaatcaaaatgtagccgcgaaggccggtgtggcagtaagtaccaatggtggcagcgcaacctataacgtcggtttaaactttgagtggtaattcacttaaagtgcggtcagaattttaggtgtttttggctgcatcaataaaacaaaacccgttttcaatgaaagcgggtttttttgtgtttcgcgattaacaattatttcgcaatgcgtttatatttaattcggtgcggtttaagggcttcagcaccgaaagttttctttttccattcttcataatcggagaaattgccttcgtagaaggtgactttgccttcatcgccgtaatctaaaatgtgggtggcgatacgatctaagaaccagcggtcatgggaaattaccatggcacagcccgggaattccaagatggcattttccagtgcgcgcaaggtttccacatcaagatcgttagttggctcgtccaataacagcatgttgccgccggcttgtaataatttcgccaaatgcaaacgaccacgttcaccgccggagagttcgcccacacgtttttgttgatccacgcctttgaagtt

SEQ ID NO: 95 - protein, 185 aa, Artificial Sequence
  Synthetic Construct
MNKKMKMFIVCAVFILIGACKIHTSYDEQSSGEINHTLYDEQSNGELKLKKIEFSKFTVKIKNKDNNSNWTDLGTLVVRKEENGIDTGLNAGGHSATFFSLKESEVNNFVKAMTEGGSFKTDEYYGYGKEQSNLDNGTSNKEIITKIEKIDGTKYITFSGNKIKDSGDKVAEYAILLEDLKKNLK

SEQ ID NO: 96 - DNA, 558 bp, Artificial Sequence
  Synthetic Construct
atgaataagaaaatgaaaatgtttattgtttgtgctgtttttatacttataggtgcttgcaaaattcatacttcatatgatgagcaaagtagtggtgagataaaccatactttatatgatgagcaaagtaatggtgagttaaaacttaaaaaaatagaattctctaaatttactgtaaaaattaaaaataaagataataatagtaactggacagacctaggaactttagttgtaagaaaagaagaaaatggtattgatacgggtttaaacgctgggggacattcggctacattcttttcattaaaagaatcagaagttaataactttgtaaaagcaatgactgaaggtggttcgtttaaaactgatgagtattatggatatggaaaggaacaaagtaatttagataatggtactagcaataaagagataataacaaagatagaaaaaattgatggaactaaatatattacattttcaggaaataaaattaaggattccggggataaagttgctgaatatgcgatactactagaagatcttaaaaaaaatttaaaatag

SEQ ID NO: 97 - protein, 158 aa, Artificial Sequence
  Synthetic Construct
MNKKMKMFIICAVFILIGACKIHTSYDEQSNGEVKVKKIEFSEFTVKIKNKNNSNNWADLGDLVVRKEKDGIETGLNAGGHSATFFSLEEEEINNFIKAMTDGGSFKTSLYYGYNDEESDKNVIKNKEIKTKIEKINDTEYITFLGDKINNSAGGDKI

SEQ ID NO: 98 - DNA, 580 bp, Artificial Sequence
  Synthetic Construct
ttttgttaaaatgtaacagctgaatgtaacaaaattatatatttaaatctttgaaatattgcatttattatgtattgtggtatgattaggacttatggagaaatttatgaataagaaaatgaaaatgtttattatttgtgctgtttttatacttataggtgcttgcaagattcatacttcatatgatgagcaaagcaatggagaggtaaaggtcaaaaaaatagaattctctgaatttactgtaaaaattaaaaataagaataatagtaataactgggcagacttaggagatttagttgtaagaaaagaaaaagatggtattgaaacgggtttaaacgctgggggacattcggctacattcttttcattagaagaggaagaaattaataactttataaaagcaatgactgacggtggatcatttaaaactagtttgtattatggatataatgacgaagaaagtgataaaaatgtcattaagaataaagagataaaaacaaagatagaaaaaattaatgatactgaatatattacatttttaggagataaaattaataacagtgcggggggagacaaaata

SEQ ID NO: 99 - protein, 386 aa, Artificial Sequence
  Synthetic Construct
MRKKLTALVLSALPLAAVADVSLYGEIKAGVEGRNIQLQLTEPLQNIQQPQVTKRKSRIRTKISDFGSFIGFKGSEDLGEGLKAVWQLEQDVSVAGGGATRWGNRESFVGLAGEFGTLRAGRVANQFDDASKAIDPWDSNNVVASQLGIFKRHDDMPVSVRYDSPEFSGFSGSVQFVPAQNSKSAYTPAHFVQQTPQSQPTLVPAVVGKPGSDVYYAGLNYKNGGFAGNYAFKYAKHANVGRDAFELFLLGSGSDEAKGTDPLKNHQVHRLTGGYEEGGLNLALAAQLDLSENADKTKNSTTEIAATASYRFGNAVPRISYAHGFDFIERGKKGENTSYDQIIAGVDYDFSKRTSAIVSGAWLKRNTGIGNYTQINAASVGLRHKF

SEQ ID NO: 100 - DNA, 1161 bp, Artificial Sequence
  Synthetic Construct
atgcgaaaaaaacttaccgcgctcgtattgtccgcactgccgcttgcggccgttgccgatgttagcctgtacggcgaaatcaaagccggcgtggaaggcaggaacatccagctgcagttgaccgaaccgctccaaaatattcaacaacctcaggttactaagcgcaaaagccgcatcaggacgaaaatcagcgatttcggctcgtttatcggctttaaggggagcgaggatttgggcgaagggctgaaggctgtttggcagcttgagcaagacgtatccgttgccggcggcggcgcgacccgttggggcaacagggaatcctttgtcggcttggcaggcgaattcggtacgctgcgcgccggccgcgttgcgaatcagtttgacgatgccagcaaagccattgatccttgggacagcaataatgttgtggcttcgcaattgggtattttcaaacgccacgacgatatgccggtttccgtacgctacgattcccccgaattttccggtttcagcggcagcgtccaattcgttccggctcaaaacagcaagtccgcctatacgccggctcattttgttcagcagactcctcaaagtcagcctactctcgttccggctgttgtcggcaagccggggtcggatgtgtattatgccggtctgaattacaaaaatggcggttttgccgggaactatgcctttaaatacgcgaaacacgccaatgtgggccgtgatgcttttgagttgttcttgctcggcagcgggagtgatgaagccaaaggtaccgatcccttgaaaaaccatcaggtacaccgcctgacgggcggctatgaggaaggcggcttgaatctcgccttggcggctcagttggatttgtctgaaaatgccgacaaaaccaaaaacagtacgaccgaaattgccgcgactgcttcctaccgcttcggtaatgcagttccacgcatcagctatgcccatggtttcgactttatcgaacgcggtaaaaaaggcgaaaataccagctacgatcaaatcatcgccggcgttgattatgatttttccaaacgcacttccgccatcgtgtctggcgcttggctgaaacgcaataccggcatcggcaactacactcaaattaatgccgcctccgtcggtttgcgccacaaattctaa

SEQ ID NO: 101 - protein, 769 aa, Artificial Sequence
  Synthetic Construct
MFKSNYERKMCYSIRKFSIGVASVAVASLVMGSVVHATENEGTTQAPTSSNRGNESQAEQRRELDLERDKVKKEVREYKEKKVKELYSKSTKSRHKKTVDIVNKLQNINNEYLNKIIQSTSTYEELQKLMMESQSEVDKAVSEFEKDLSSSSSSGSSTEPEASDTAKPNKPTELEKKVAEAQQKVEEAEKKAKDQKEEDYRNYPTITYKTLELEIAEFDVKVKEAELELVKVKAKESRDEKKIKQAEAEVESKQAEATRLKKIKTDRKKAEEEAKLKEAVEKNAATSEQGKPKRRVKRGALGEQATPDKKDYFEKDFRPAFNKNQQMVAIQESLNKLDGETKTVPDGAKLTGEAGNAYNEVRDYAIKVVSENKKLLSQTAVTMDELAMQLTKLNDAMSKLREAKAKLVPEVKPQPENPEHQRPTTPAPDTKPIPQPEGKKPSVPDINQEKEKAKLAVATYMSKILDDIQKHHLQKEKHRQIVALIKELDEFKKQALSEIDNVNTKVEIENTVHKIFADMDAVVTKFKKGLTQDTPKEPDNKKPSAPKPGMQPSPQPEGKKPSVPAQPGTEDKKPSAPKPGMQPSPQPEGKKPSVPAQPGTEDKKPSAPKPDMQPSPQPEGKKPSVPAQPGTEDKKPSAPKPGMQPSPQPEGKKPSVPAQPGTEDKKPSAPKPDMQPSPQPEGKKPSVPAQPGTEDKKPSAPKPDMQPSPQPEGKKPSVPEINQEKEKAKLAVATEKKLPSTGVASNLVLEIIGLLGLIGTSFIAMKRRK

SEQ ID NO: 102 - DNA, 2310 bp, Artificial Sequence
  Synthetic Construct
atgtttaaatcaaattatgaaagaaaaatgtgctattctattcgaaaatttagtataggagtagctagtgtagctgttgccagtcttgttatgggaagtgtggttcatgcgacagagaacgagggaactacccaagcacccacttcttctaataggggaaatgaaagtcaggcagaacaacgtagagaactcgatttagaacgagataaggtaaagaaagaggtcagggaatataaagaaaaaaaagtgaaagagctctattcaaaatcaactaaaagtcgacataagaagactgtagatatagttaacaagttgcaaaacattaataacgagtatttgaataaaataattcaatcaacctcaacatacgaagaactacagaaactgatgatggagagtcaatcagaagtagataaagctgtgtctgaatttgaaaaggacttatcttcttcgtcaagttcaggctcttccacggaaccggaagcttcagatacagcgaagccaaacaagccgacagaactagaaaaaaaggtagcagaagctcagcagaaggttgaagaagctgagaaaaaagccaaggatcaaaaagaagaagattaccgtaactacccaaccattacttacaaaacgcttgaacttgaaattgctgagttcgatgtgaaagttaaagaagcggagcttgaactagtaaaagtgaaagctaaggaatctcgagacgagaaaaaaattaagcaagcagaagcggaagttgagagtaaacaagctgaggctacaaggttaaaaaaaatcaagacagatcgtaaaaaagcagaagaagaagctaagttgaaggaagctgttgaaaagaatgcagcgacttcagagcaaggtaaaccaaagaggcgggtaaaacgaggagctcttggagagcaagcaacacctgataaaaaagattattttgaaaaagacttccgtccagctttcaataaaaaccagcagatggtagccattcaagaatccttgaacaaactagatggtgaaacaaaaactgttccagatggggctaaactcacaggagaagctggaaatgcctataatgaggtcagagattatgcaataaaagttgtttctgaaaacaagaaacttctatcacagacagcagtgacaatggatgaactggcaatgcaattaaccaaattgaacgatgccatgtctaaattgagagaggctaaagcgaaattggtaccagaggttaaaccgcagccggaaaatccagagcatcaaagaccaacaactccagctccggataccaaaccaatccctcaaccagagggtaagaaaccaagcgtaccagatattaatcaggaaaaagaaaaagctaagcttgctgtagcaacctacatgagcaagattttagatgatatacaaaaacatcatctgcagaaagaaaaacatcgtcagattgttgctcttattaaggagcttgatgagtttaaaaagcaagctctttctgaaattgataatgtaaataccaaagtagaaattgaaaatacagtccacaagatatttgcagacatggatgcagttgtgactaaattcaaaaaaggcttaactcaggacacaccaaaagaaccagataacaaaaagccatctgctccaaaaccaggtatgcaaccaagtcctcaaccagaaggcaagaaaccaagcgtaccggcacaacctggtactgaggataaaaagccatctgctccaaaaccaggtatgcaaccaagtcctcaaccagaaggcaagaaaccaagcgtaccggcacaacctggtactgaggataaaaagccatctgctccaaaaccagatatgcaaccaagtcctcaaccagaaggcaagaaaccaagcgtaccggcacaacctggtactgaggataaaaagccatctgctccaaaaccaggtatgcaaccaagtcctcaaccagaaggcaagaaaccaagcgtaccggcacaacctggtactgaggataaaaagccatctgctccaaaaccagatatgcaaccaagtcctcaaccagaaggcaagaaaccaagtgtaccggcacaacccggtactgaggataaaaagccatctgctccaaaaccagatatgcaaccaagccctcaaccagagggtaagaaaccaagtgtaccagagattaatcaggagaaagaaaaagctaagcttgctgtagcaacagagaagaaattgccatctacaggagtggcgtctaatctagttcttgagatcattggtctccttggtttgattggaacttcattcatcgcgatgaaaagaagaaaataa

SEQ ID NO: 103 - protein, 163 aa, Artificial Sequence
  Synthetic Construct
MKKLVLARAPALVLTGATVSTGTVSANSSRYNYTGWNQGGYSWKYLRLRNKNPYSRRTLTEDYSDQRKNEAKDSIKELSKLRDKEKKNFADRIDALTDTYAISSILSEAKNKNNDYLEFDKEYEALFNSNKYKLEIEKIKDRVYFDEGYSARQGINDLKSLEN

SEQ ID NO: 104 - DNA, 683 bp, Artificial Sequence
  Synthetic Construct
tacatgagtggttattcttgttttgtaacacgctattaaaactgatattgaagaagcttgaagataatagtaaaataaaaccctaaacttatttaataaatggaggatttttcaacgtgaaaaaattagttttagcccgtgcaccagcacttgtattaacaggagcaactgtaagtacaggaacagtaagtgcgaatagttcaagatataattataccggttggaatcaaggtgggtactcatggaaatatttgcgcctaagaaataaaaatccttattcacgtcgtacccttacagaagactattctgatcaaagaaagaatgaagcaaaagatagtattaaagaattgagtaagcttagagataaagagaaaaagaattttgctgatagaattgatgctttgactgacacctatgcaatcagctctattctatcagaagctaaaaataagaataatgactatttagaatttgataaagaatatgaggctttattcaattcaaataagtataagttagaaatagagaaaataaaggatcgtgtgtactttgatgaaggttactcagctagacaagggattaatgacttgaaaagtctggagaattaaacagtagacagatttattatttatcaaaataatccaactgaaagtctatgttttaaggaagcttattaaggtata

SEQ ID NO: 105 - protein, 395 aa, Artificial Sequence
  Synthetic Construct
MAKEKFERNKPHVNVGTIGHVDHGKTTLTAALTKVCSDTWGGSARAFDQIDNAPEEKARGITINTSHVEYDSAVRHYAHVDCPGHADYVKNMITGAAQMDGAILVCSAADGPMPQTREHILLSRQVGVPYIVVFLNKADMVDDAELLELVEMEVRDLLNTYDFPGDDTPIIIGSALMALEGKDDNGIGVSAVQKLVETLDSYIPEPVRAIDQPFLMPIEDVFSISGRGTVVTGRVERGIIKVQEEVEIVGIKATTKTTCTGVEMFRKLLDEGRAGENVGILLRGTKREDVERGQVLAKPGTIKPHTKFECEVYVLSKEEGGRHTPFFKGYRPQFYFRTTDVTGNCELPEGVEMVMPGDNIKMVVTLIAPIAMEDGLRFAIREGGRTVGAGAKIIE

SEQ ID NO: 106 - DNA, 1194 bp, Artificial Sequence
  Synthetic Construct
gtggctaaggaaaaattcgaacgtaacaaaccgcacgtcaacgtcggcaccatcggtcacgttgaccatggcaagaccactctgaccgctgcactgaccaaggtctgctccgacacctggggtggttccgctcgtgctttcgatcagatcgacaacgcgccggaagaaaaggcccgcggtatcaccatcaacacctcgcacgttgaatacgattccgctgttcgtcactacgcccacgttgactgccccggtcacgccgactacgtgaagaacatgatcaccggtgctgcccagatggacggcgcgatcctggtttgctcggctgccgacggccccatgccgcagacccgcgagcacatcctgctgtcccgccaggtaggcgttccctacatcgtcgtgttcctgaacaaggctgacatggtcgacgacgccgagctgctggaactggtcgagatggaagttcgcgatctgctgaacacctacgacttcccgggcgacgacactccgatcatcatcggttccgcgctgatggcgctggaaggtaaagatgacaacggcatcggcgtaagcgccgtgcagaagctggtagagaccctggactcctacattccggagccggttcgtgccatcgaccagccgttcctgatgccgatcgaagacgtgttctcgatctccggccgcggtaccgtggtaaccggtcgtgtagagcgcggcatcatcaaggtccaggaagaggtggaaatcgtcggcatcaaggcgaccaccaagaccacctgcaccggcgttgaaatgttccgcaagctgctcgacgaaggtcgtgctggtgagaacgttggtatcctgctgcgcggcaccaagcgtgaagacgtagagcgtggccaggtactggccaagccgggcaccatcaagccgcacaccaagttcgagtgcgaagtgtacgtgctgtccaaggaagaaggtggtcgtcacactccgttcttcaagggctaccgtccgcagttctacttccgtaccaccgacgtgaccggtaactgcgagctgccggaaggcgtagagatggtaatgccgggcgacaacatcaagatggttgtcaccctgatcgctccgatcgccatggaagatggcctgcgcttcgcgatccgcgaaggcggccgtaccgttggcgccggcgtggttgccaagatcatcgagtaa

SEQ ID NO: 107 - protein, 422 aa, Artificial Sequence
  Synthetic Construct
MTKDFKISVSAALISALFSSPYAFANNDEVHFTAVQISPNADPDSHVVIFQPAAEALGGTNALAKSIHSIAVGASAEAAKQAAVAVGAGSIATGVNSVAIGPLSKALGDSAVTYGAASTAQKDGVAIGARAFTSDTGVAVGFNSKVDAKNSVAIGHSSHVAVDHDYSIAIGDRSKTDRKNSVSIGHESLNRQLTHLAAGTKDTDAVNVAQLKKEIEKTQVNANKKSAEVLGIANNYTDSKSAETLENARKEAFDLSNDALDMAKKHSNSVARTTLETAEEHTNKKSAETLARANVYADSKSSHTLQTANSYTDVTVSNSTKKAIRESNQYTDHKFRQLDNRLDKLDTRVDKGLASSAALNSLFQPYGVGKVNFTAGVGGYRSSQALAIGSGYRVNESVALKAGVAYAGSSDVMYNASFNIEW

SEQ ID NO: 108 - DNA, 1269 bp, Artificial Sequence
  Synthetic Construct
atgactaaagattttaagatcagtgtctctgcggcattaatatctgcgttgttctcatctccatatgcatttgccaataatgacgaggttcattttacagcagttcaaataagcccaaatgctgatcctgattcgcatgttgtgatatttcaaccagcagcagaagccctaggcgggaccaatgctctcgctaagagtatccatagcattgcggttggtgctagtgctgaagcagcgaaacaagctgcagttgctgtgggcgctggttcaattgcaacaggagttaattctgttgcaattggtcctttaagtaaggcattgggagattcggcagttacttatggggcagctagtaccgcccagaaagatggagtagctatcggtgcgagagcattcacttcagatactggtgtcgctgtcggttttaactcgaaagttgatgcaaaaaactctgttgccattggacactctagtcacgttgcggtagatcatgattattcaattgcaattggggatcgttctaaaactgaccgaaaaaatagtgtatccattggtcatgaaagccttaatcgccaattgacacatcttgcggctggcactaaagacactgatgcagtgaatgtcgcgcaattaaagaaagaaattgaaaaaacacaggtaaatgcaaataaaaaatcagctgaggtgctagggatcgcaaataactatactgatagtaaaagtgctgaaacattggaaaatgcgcgtaaagaggcttttgacctgtctaacgatgctttggatatggcaaaaaaacactcaaatagtgttgctagaacaactttagaaactgctgaagaacatacaaataaaaaatcagctgagacgttagcaagagctaatgtgtatgcagacagcaagtcttctcacacactacaaactgcaaatagctataccgatgtgactgtaagtaattcgactaagaaagcaatccgtgaatcgaatcaatacacagatcataaattccgtcaacttgacaaccgtttagataaacttgacacacgagttgacaaaggtttagccagttcagccgctttaaacagcttgttccagccatatggtgtggggaaagtaaactttactgcaggtgtcgggggatatcgctctagtcaggcattagcaattggttctggctatcgtgtaaatgagagtgtcgcacttaaagccggtgtggcttatgccggttcctcggatgtcatgtacaatgcatcatttaatatcgagtggtaa

(19) United States
(12) Patent Application Publication
(10) Pub. No.: US 2011/0300171 A1
(43) Pub. Date: Dec. 8, 2011

(54) FACTOR H BINDING PROTEIN IMMUNOGENS

(75) Inventors: Silvana Savino, Siena (IT); John Donnelly, Morega, CA (US); Rino Rappuoli, Siena (IT); Mariagrazia Pizza, Siena (IT); Barbara Bottazzi, Rozzano (IT)

(73) Assignees: HUMANITAS MIRASOLE S.P.A., Rozzano (IT); NOVARTIS VACCINES AND DIAGNOSTICS S.R.L., Siena (IT)

(21) Appl. No.: 13/063,458

(22) PCT Filed: Sep. 14, 2009
(86) PCT No.: PCT/EP2009/006651

§ 371 Date: Aug. 19, 2011

(30) Foreign Application Priority Data
Sep. 12, 2008 (IT) MI2008A 001633

(51) Int. Cl. A61K 39/095; A61P 37/04; A61P 31/04; A61K 39/00

(52) U.S. Cl. 424/190.1; 424/249.1; 424/250.1; 424/184.1

(57) ABSTRACT

The invention relates to immunization against pathogenic bacterial strains which express or can express multiple factor H binding proteins. Certain aspects of the invention include vaccine compositions comprising at least two factor H binding proteins derived from a pathogenic bacterial strain which expresses multiple facto H binding proteins.

### TECHNICAL FIELD

This invention relates to immunization against pathogenic bacterial strains which express or can express multiple factor H binding proteins.

### BACKGROUND ART

Reverse vaccinology is a novel paradigm for generation of vaccines to bacterial pathogens pioneered by Rino Rappuoli and others. In reverse vaccinology, one scans the genome of a pathogen of interest for promising antigens, identifying antigens capable of generating a bactericidal response to the pathogen and then further narrowing the list of possible antigens by identifying which are well conserved across multiple strains of the pathogen to give as complete as possible coverage. The first success in reverse vaccinology was in the development of a multicomponent, recombinant-protein-based vaccine against N. meningitidis serogroup B (See M. Giuliani et al., PNAS (2006) 103(29):10834-10839). Identification and screening of likely candidates that can provide the broadest possible coverage across multiple strains is a time consuming endeavor. Thus, there is a need for improved method of identification of such strong candidates and for multicomponent vaccines comprising such strong candidates.

One such candidate is the meningococcal factor H binding protein (fHBP), also known as protein ‘741’, ‘NNMB 1870’, GNA 1870’ [refs. N6, N10, N21]. This lipoprotein is expressed across all meningococcal serogroups and has been found in multiple meningococcal strains. NMB 1870 has been identified to be a ligand for factor H, an inhibitor of the alternative complement pathway [ref. N22, N23]. fHBP has been shown to induce antibodies that have both complement-mediated bacterial killing activity and that inhibit binding of factor H to the bacterial surface, increasing the susceptibility of bacteria to the lysis by human complement [ref. N21]. fHBP is important for bacterial survival in human blood, human serum and in the presence of antimicrobial peptides.

Thus, it is an object of the invention to provide improved multicomponent vaccines comprising two or more factor H binding polypeptides against pathogens that provide a broad protection against a range of pathogen strains.

It is a further object of the invention to provide methods of screening antigen candidates for superior range of protection by assaying for factor H binding activity.

### DISCLOSURE OF THE INVENTION

For the purpose of the present invention, the term “factor H binding” refers to the capacity to bind factor H, identified and measured by the methods and standards described in refs. N22 and N23. The following are representative factor H binding proteins from a range of pathogens of interest that may be used in the polypeptide combinations of the present invention.

### NMB1870 Protein

NMB 1870 protein from serogroup B is disclosed in reference N6 (see also GenBank accession number GI: 7227128) and as ‘741’ in reference N10 (SEQ IDs 2535 & 2536). The corresponding protein in serogroup A (N5) has GenBank accession number 7379322.741 is naturally a lipoprotein.

When used according to the present invention, NMB 1870 protein may take various forms. Preferred forms of NMB 1870 are truncation or deletion variants, such as those disclosed in references N14 to N16. In particular, the N-terminus of NMB 1870 may be deleted up to and including its poly-glycine sequence (i.e. deletion of residues 1 to 72 for strain MC58). This deletion can enhance expression. The deletion also removes NMB 1870's lipidation site.

Preferred NMB1870 sequences have 50% or more identity (e.g. 60%, 70%, 80%, 90%, 95%, 99% or more) to SEQ ID 1. This includes NMB1870 variants (e.g. allelic variants, homologs, orthologs, paralogs, mutants, etc.). Allelic forms of NMB1870 can be found in SEQ IDs 1 to 22 of reference N16, and in SEQ IDs 1 to 23 of reference N19. SEQ IDs 1-299 of reference N20 give further. NMB 1870 sequences.

Other preferred NMB1870 sequences comprise at least n consecutive amino acids from SEQ ID 1, wherein n is 7 or more (e.g. 8, 10, 12, 14, 16, 18, 20, 25, 30, 35, 40, 50, 60, 70, 80, 90, 100, 150, 200, 250 or more). Preferred fragments comprise an epitope from NMB1870. Other preferred fragments lack one or more amino acids (e.g. 1, 2, 3, 4, 5, 6, 7, 8, 9, 10, 15, 20, 25 or more) from the C-terminus and/or the N-terminus of SEQ ID 1.

Protein NMB1870 is an extremely effective antigen for eliciting anti-meningococcal antibody responses, and it is expressed across all meningococcal serogroups. Phylogenetic analysis shows that the protein splits into two groups, and that one of these splits again to give three variants in total (N21), and while serum raised against a given variant is bactericidal within the same variant W group, it is not active against strains which express one of the other two variants i.e., there is intra-variant cross-protection, but not inter-variant cross-protection. For maximum cross-strain efficacy, therefore, it is preferred that a composition should include more than one variant of protein NMB 1870.

### NMB2091 Protein

NMB2091 protein from serogroup B is disclosed in reference N6 (see also GenBank accession number GI: 7227353) and as ‘936’ in reference N10 (SEQ IDs 2883 & 2884). The corresponding gene in serogroup A (N5) has GenBank accession number 7379093.

When used according to the present invention, NMB2091 protein may take various forms. Preferred forms of NMB2091 are truncation or deletion variants, such as those disclosed in references N14 to N16. In particular, the N-terminus leader peptide of NMB2091 may be deleted (i.e., deletion of residues 1 to 23 for strain MC58 (SEQ ID 41)) to give NMB2091 (NL).

Preferred NMB2091 sequences have 50% or more identity (e.g. 60%, 70%, 80%, 90%, 95%, 99% or more) to SEQ ID 41. This includes variants (e.g. allelic variants, homologs, orthologs, paralogs, mutants etc).

Other preferred NMB2091 sequences comprise at least n consecutive amino acids from SEQ ID 41, wherein n is 7 or more (e.g. 8, 10, 12, 14, 16, 18, 20, 25, 30, 35, 40, 50, 60, 70, 80, 90, 100, 150, 200, 250 or more). Preferred fragments comprise an epitope from NMB2091. Other preferred fragments lack one or more amino acids (e.g. 1, 2, 3, 4, 5, 6, 7, 8, 9, 10, 15, 20, 25 or more) from the C-terminus and/or the N-terminus of SEQ ID 41.

### NMB1030 Protein

NMB1030 protein from serogroup B is disclosed in reference N6 (see also GenBank accession number GI: 7226269) and as 953 in reference 10 (SEQ IDs 2917 & 2918). The corresponding protein in serogroup A (N5) has GenBank accession number 7380108.

When used according to the present invention, NMB1030 protein may take various forms. Preferred forms of NMB1030 are truncation or deletion variants, such as those disclosed in references N14 to N16. In particular, the N-terminus leader peptide of NMB1030 may be deleted (i.e. deletion of residues 1 to 19 for strain MC58 (SEQ ID 11)) to give NMB1030 (NL).

Preferred NMB1030 sequences have 50% or more identity (e.g 60%, 70%, 80%, 90%, 95%, 99% or more) to SEQ ID 11. This includes NMB1030 variants (e.g. allelic variants, homologs, orthologs, paralogs, mutants, etc.). Allelic forms of NMB 1030 can be seen in FIG. 19 of reference N12.

Other preferred NMB1030 sequences comprise at least n consecutive amino acids from SEQ ID 11, wherein n is 7 or more (e.g. 8, 10, 12, 14, 16, 18, 20, 25, 30, 35, 40, 50, 60, 70, 80, 90, 100, 150, 200, 250 or more). Preferred fragments comprise an epitope from NMB1030. Other preferred fragments lack one or more amino acids (e.g. 1, 2, 3, 4, 5, 6, 7, 8, 9, 10, 15, 20, 25 or more) from the C-terminus and/or the N-terminus of SEQ ID 11.

### NMB0667

NMB0667, hypothetical protein, has GenBank accession number 902778. A representative NMB0667 is provided as SEQ ID NO: 51, with homologs N. meningitidis serogroups A and C (SEQ ID NOs: 53, 55, and 57) and from N gonnhoroeae (SEQ ID NO: 59).

### NEISSERIAL REFERENCES

N1. Maiden et al. (1998) PNAS USA 95:3140-3145.
N2. Armand et al. (1982) J. Biol. Stand. 10:335-339.
N3. Cadoz et al. (1985) Vaccine 3:340-342.
N4. Bjune et al. (1991) Lancet 338(8775):1093-96
N5. Parkhill et al. (2000) Nature 404:502-506.
N6. Tettelin et al. (2000) Science 287:1809-1815.
N7. WO00/66791.
N8. WO99/24578.
N9. WO99/36544.
N10. WO99/57280.
N11. WO00/22430.
N12. WO00/66741.
N13. Pizza et al. (2000) Science 287:1816-1820.
N14. WO01/64920.
N15. WO01/64922.
N16. WO03/020756.
N17. Comanducci et al. (2002) J. Exp. Med. 195:1445-1454.
N18. WO03/010194.
N19. UK patent application 0227346.4.
N20. WO03/063766.
N21. Masignani et al. (2003) J Exp Med 197:789-799.
N22. Madico et al. (2006) J. Immunol., 177, 501-10
N23. Schneider et al. (2006) J. Immunol., 176, 7566-75.

### Por1A

Porin (Por) is the major outer membrane protein in Neisseria gonorrhoeae and occurs in two primary immunochemical classes, PorlA and PorlB (J. Infect. Dis. 1984, 150: 44-48). PorlA is the acceptor molecule for factor H, and strains expressing hybrid Por1A/B molecules have been used to localize the factor H binding site to loop 5 of Por1A (J Exp Med. 1998 Aug. 17; 188(4):671-80. A representative Por1A is provided in SEQ ID NO: 99 which can also be used to identify homologs in all related species.

### Omp100 (Actinobacillus spp.)

Omp100, a major outer membrane protein of Actinobacillus actinomycetemcomitans Y4, has homology to a number of virulence factors, including YadA of Yershinia enterocolitica. Omp100 is randomly localized on the cell surface of A. actinomycetemcomitans and binds to factor H (Mol Microbiology 2003, 50(4): 1125-1139). A representative Omp100 is provided in SEQ ID NO: 93 which can also be used to identify homologs in all related species.

### Complement Regulator-Acquiring Surface Proteins (CRASPS) (Borrelia spp.)

Complement regulator-acquiring surface proteins (CRASPS) promote serum resistance of Borrelia species through binding to factor H (J Biol Chem 2004, 279: 2421-2429). CRASP-1, -2, -3, -4, and -5 bind to short consensus repeat (SCR) domains of factor H with high affinity. The C-terminus of several CRASPs has been shown to be required for this binding (Mol J Immunol 2006, 43: 31-44). In particular, the factor H-binding site of BbCRASP-3 has been localized to the nine amino acid sequence, LEVLKKNLK, of the C-terminus of this protein (Eur J Immunol 2203, 33:697-707). Representative CRASPS are provided in SEQ ID NO: 63 and 65 which can also be used to identify homologs in all related species.

### OspE/F-Related Protein (ERP) Family (Borrelia spp.)

Genes encoding Erp proteins are present in all Lyme disease Borrelia species. Erp proteins localize to the bacterial outer surface and are expressed upon mammalian infection (Microbiology 2001, 147: 821-830; J Mol Microbiol Biotechnol 2000, 2: 411-422). Most Erp proteins, including OspE, p21/orf28, ErpA (BBL39), ErpC, and ErpP (BBN38), bind to Factor H (Infection and Immunity 2002, 70(2): 491-497; Mol Immunol 2006, 43: 31-44). These proteins generally bind to SCRs 19-20 of factor H through their C-terminus. Representative erps are provided in SEQ ID NO: 97, 73, 75, and 77 which can also be used to identify homologs in all related species.

### FHBP19/FhbA and FHBP28

Two factor H binding proteins have been identified in Borrelia hermsii (J Clin Microbiol 2003, 41: 3905-3910; J Bacteriol 2004, 186: 2612-2618). FHBP19/FhbA is a 19 kDa protein and shows no homology to CRASPs or other spirochaetal factor H binding proteins. FHBP28 is a 28 kDa protein. A representative FhbA is provided in SEQ ID NO: 85 which can also be used to identify homologs in all related species.

LfhA (Leptospira interrogans)

Leptospira factor H-binding protein A (LfhA) was identified by screening a lambda expression library of L. interrogans for clones that bound factor H (Infect Immun 2006, 74: 2659-2666). Ligand affinity blot assays with recombinant LfhA confirmed its ability to bind factor H. LfhA is expressed during mammalian infection and localizes to outer and inner membranes. A representative LfhA is provided in SEQ ID NO: 91 which can also be used to identify homologs in all related species.

### Tuf (Pseudomonas spp.)

Elongation factor Tuf was isolated from Pseudomonas aeruginosa as a factor H binding protein with a factor H affinity matrix and mass spectrometry (J Immunol 2007, 179: 2979-2988). Tuf localizes to the surface of P. aeruginosa. Binding of Tuf to factor H is mediated through SCR domains 6-7 and 19-20 in factor H. A representative Tuf is provided in SEQ ID NO: 105 which can also be used to identify homologs in all related species.

### Bac (Streptococcus spp.)

Bac or β protein is a surface protein of group B streptococcus. Bac was shown to bind factor H through mutational analysis as well as binding experiments with recombinant proteins (J Biol Chem 2002, 277: 12642-12648). Bac and heparin compete for binding to factor H within SCR 13 or 20, and the C-terminus of Bac is also required for binding (Mol Immunol 2006, 43: 31-44). A representative Bac is provided in SEQ ID NO: 61 which can also be used to identify homologs in all related species.

### Fba (Streptococcus spp.)

Fba was the first non-M-like protein of group A streptococcus shown to bind to human regulators of complement activity, including factor H (Infect Immun 2002, 70: 6206-6214). Terao et al identified the same protein as a fibronectin binding protein involved in invasion of Hep-2 cells (Mol Microbiol 2001, 42: 191-199). An N-terminal region of Fba predicted to contain a coiled-coil is required for binding to factor H, and the Fba binding site of factor H was localized to SCR 7 (Infec Immun 2003, 71:7119-7128). A representative Fba is provided in SEQ ID NO: 79 which can also be used to identify homologs in all related species.

### Hic (Streptococcus spp.)

Factor H-binding inhibitor of complement (Hic) gene encodes a novel surface protein in the pspC locus of type 3 pnuemococci (J Biol Chem 2000, 275: 37257-37263). Hic has low overall sequence homology to other PspC proteins. The N-terminal helical region (amino acids 39-261) of Hic is required for its binding to factor H. SCRs 8-11 and 12-14 on factor H are also required for binding.

### M/emm Proteins (Streptococcus spp.)

Comparison of M+ and M− strains of Streptococcus pyogenes first demonstrated that factor H binds to the cell surface of M+ strains (PNAS 1988, 85: 1657-1661). Specific binding between emm5, emm6, and emm18 has been demonstrated. All three bind to SCR7 of factor H (Mol Immunol 2006, 43: 31-44). A representative M protein homologs are provided in SEQ ID NO: 67, 69, and 71 which can also be used to identify homologs in all related species.

### PspC (Streptococcus spp.)

Members of the PspC family attach to the cell surface through a C-terminal anchor. They contain a conserved 37 amino acid leader peptide and an N-terminal α-helical domain followed by a proline-rich region (Gene 2002, 284: 63-71). The factor H binding site on PspC was mapped to the N-terminal α-helical region (amino acids 1-225), and the PspC binding site on factor H was mapped to SCRs 13-15 (Indian J Med Res 2004, 119(Suppl,): 66-73; Infect Immun 2002, 70: 5604-5611). Representative PspCs are provided in SEQ ID NO: 89 and 101 which can also be used to identify homologs in all related species.

Se18.9 (Streptococcus equi)

Se18.9 is a novel surface bound protein secreted by S. equi but not S. zooepidemicus (Vet Microbiol 2007, 121: 105-115). Se18.9 binds to factor H and is immunoreactive with convalescent sera and mucosal IgA. A representative Se 18.9 is provided in SEQ ID NO: 103 which can also be used to identify homologs in all related species.

### YadA (Yersinia spp.)

YadA is a polymer of about 200 kDa formed of 47 kDa subunits that forms a fibrillar structure at the surface of Yersinia enterocolitica (EMBO J 1985, 4: 1013-1018). Western blot analysis demonstrated that YadA binds to factor H (Infect Immun 1993, 61: 3129-3136). A representative YadA is provided in SEQ ID NO: 107 which can also be used to identify homologs in all related species.

Gpm1p (Candida albicans)

Gpm1p was the first fungal protein identified to bind to host complement regulators. CaGPM1p is a surface protein that binds to two regions in factor H, SCRs 6 and 7 and SCRs 19 and 20 (J Biol Chem 2007, 282: 37537-37544). A representative CaGMP1p from S. cerevisiae is provided in SEQ ID NO: 87 which can be used to identify homologs in all fungal pathogens.

Polypeptides Used with the Invention

The invention provides combinations of two or more polypeptides comprising an amino acid sequence that (in each case selected from different non-homologous sequences and not NMB 1870 and NMB1030, NMB1870 and NMB2091, or NMB1030 and NMB2091 if only two polypeptides):
(a) is identical (i.e. 100% identical) to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(b) has at least a % sequence identity to one or more of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(c) is a fragment of at least b consecutive amino acids of one or more of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(d) has 1, 2, 3, 4, 5, 6, 7, 8, 9 or 10 (or more) single amino acid alterations (deletions, insertions, substitutions), which may be at separate locations or may be contiguous, as compared to the sequences, of (a) or (b); and/or
(e) when aligned with any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107 using a pairwise alignment algorithm, each moving window of x amino acids from N-terminus to C-terminus (such) that for an alignment that extends to p amino acids, where p>x, there are p−x+1 such windows) has at least x·y identical aligned amino acids, where: x is selected from 20, 25, 30, 35, 40, 45, 50, 60, 70, 80, 90, 100, 150, 200; y is selected from 0.50, 0.60, 0.70, 0.75, 0.80, 0.85, 0.90, 0.91, 0.92, 0.93, 0.94, 0.95, 0.96, 0.97, 0.98, 0.99; and if xy is not an integer then it is rounded up to the nearest integer. The preferred pairwise alignment algorithm is the Needleman-Wunsch global alignment algorithm (1), using default parameters (e.g. with Gap opening penalty=10.0, and with Gap extension penalty=0.5, using the EBLOSUM62 scoring matrix). This algorithm is conveniently implemented in the needle tool in the EMBOSS package (2).

These polypeptides include variants of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107, including allelic variants, polymorphic forms, homologs, orthologs, paralogs, mutants, etc.

The value of a may be selected from 50%, 60%, 65%, 70%, 75%, 80%, 85%, 87.5%, 90%, 91%, 92%, 93%, 94%, 95%, 96%, 97%, 98%, 99%, 99.5% or more.

The value of b may be selected from 7, 8, 9, 10, 12, 14, 16, 18, 20, 25, 30, 35, 40, 50, 60, 70, 80, 90, 100, 150, 200, 250 or more. Preferred fragments of comprise an epitope from SEQ ID NOs SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107. Other preferred fragments lack one or more amino acids (e.g. 1, 2, 3, 4, 5, 6, 7, 8, 9, 10, 15, 20, 25 or more) from the C-terminus and/or one or more amino acids (e.g. 1, 2, 3, 4, 5, 6, 7, 8, 9, 10, 15, 20, 25 or more) from the N-terminus of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107, preferably while retaining at least one epitope of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107. Other fragments omit One or more protein domains e.g. omission of a signal peptide, of a cytoplasmic domain, of a transmembrane domain, of an extracellular domain, etc.

An epitope within a fragment may be a B-cell epitope and/or a T-cell epitope. Such epitopes can be identified empirically (e.g. using PEPSCAN (3,4) or similar methods), or they can be predicted (e.g. using the Jameson-Wolf antigenic index (5), matrix-based approaches (6), MAPITOPE (7), TEPITOPE (8,9), neural networks (10), OptiMer & EpiMer (11, 12), ADEPT (13), Tsites (14), hydrophilicity (15), antigenic index (16) or the methods disclosed in references 17-21, etc.). Epitopes are the parts of an antigen that are recognised by and bind to the antigen binding sites of antibodies or T-cell receptors, and they may also be referred to as “antigenic determinants”.

A polypeptide of the invention for use in these combinations may, compared to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107, include one or more (e.g. 1, 2, 3, 4, 5, 6, 7, 8, 9, etc.) amino acid substitutions, such as conservative substitutions (i.e. substitutions of one amino acid with another which has a related side chain). Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity.

A polypeptide may include one or more (e.g. 1, 2, 3, 4, 5, 6, 7, 8, 9, etc.) single amino acid deletions relative to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107. Similarly, a polypeptides may include one or more (e.g. 1, 2, 3, 4, 5, 6, 7, 8, 9, etc.) insertions (e.g. each of 1, 2, 3, 4 or 5 amino acids) relative to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107.

Within group (c), deletions or substitutions may be at the N-terminus and/or C-terminus, or may be between the two termini. Thus a truncation is an example of a deletion. Truncations may involve deletion of up to 40 (or more) amino acids at the N-terminus and/or C-terminus.

In general, when a polypeptide of the invention comprises a sequence that is not identical to a complete one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107 (e.g. when it comprises a sequence listing with <100% sequence identity thereto, or when it comprises a fragment thereof) it is preferred that the polypeptide can elicit an antibody that recognises a polypeptide consisting of the complete SEQ ID sequence i.e. the antibody binds to an epitope in one or more of said SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107.

In one embodiment, the invention provides a polypeptide comprising an amino acid sequence: (a) having at least a % identity to any one of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107; and (b) comprising a fragment of at least b consecutive amino acids of said SEQ ID.

A polypeptide of the invention may include a metal ion e.g. a metal ion that is coordinated by one or more amino acids in the polypeptide chain. For instance, the polypeptide may include a monovalent, divalent or trivalent metal cation. Divalent cations are typical, such as Mn²⁺, Fe²⁺, Co²⁺, Ni²⁺, Cu²⁺, etc. The divalent cation is preferably Zn²⁺. The ion may be coordinated by a HEAGH or HEVGH amino acid sequence.

Polypeptides used with the invention can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.).

Polypeptides used with the invention can be prepared by various means (e.g. recombinant expression, purification from cell culture, chemical synthesis, etc.). Recombinantly-expressed proteins are preferred.

Polypeptides used with the invention are preferably provided in purified or substantially purified form i.e. substantially free from other polypeptides (e.g. free from naturally-occurring polypeptides), particularly from other polypeptides from the pathogen of interest or host cell polypeptides, and are generally at least about 50% pure (by weight), and usually at least about 90% pure i.e. less than about 50%, and more preferably less than about 10% (e.g. 5%) of a composition is made up of other expressed polypeptides. Thus the antigens in the compositions are separated from the whole organism with which the molecule is expressed.

Polypeptides used with the invention are preferably factor H binding polypeptides.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included are, for example, polypeptides containing one or more analogs of an amino acid (including, for example, unnatural amino acids, etc.), as well as other modifications known in the art. Polypeptides can occur as single chains or associated chains.

The invention provides polypeptides comprising a sequence —P-Q- or -Q-P—, wherein: —P— is an amino acid sequence as defined above and -Q- is not a sequence as defined above i.e. the invention provides fusion proteins. Where the N-terminus codon of —P— is not ATG, but this codon is not present at the N-terminus of a polypeptide, it will be translated as the standard amino acid for that codon rather than as a Met. Where this codon is at the N-terminus of a polypeptide, however, it will be translated as Met. Examples of -Q- moieties include, but are not limited to, histidine tags (i.e. Hisₙ where n=3, 4, 5, 6, 7, 8, 9, 10 or more), a maltose-binding protein, or glutathione-S-transferase (GST).

The invention also provides an oligomeric protein comprising a polypeptide of the invention. The oligomer may be a dimer, a trimer, a tetramer, etc. The oligomer may be a homo-oligomer or a hetero-oligomer. Polypeptides in the oligomer may be covalently or non-covalently associated.

The invention also provides a process for producing a polypeptide of the invention, comprising the step of culturing a host cell transformed with nucleic acid of the invention under conditions which induce polypeptide expression. The polypeptide may then be purified e.g. from culture supernatants.

The invention provides a host cell, containing a plasmid that encodes a polypeptide of the invention. The chromosome of the host cell may include a homolog of the factor H binding polypeptide, or such a homolog may be absent, but in both cases the polypeptide of the invention can be expressed from the plasmid. The plasmid may include a gene encoding a marker, etc. These and other details of suitable plasmids are given below.

Although expression of the polypeptides of the invention may take place in the strain from which the polypeptide was derived, the invention will usually use a heterologous host for expression. The heterologous host may be prokaryotic (e.g. a bacterium) or eukaryotic. Suitable hosts include, but are not limited to, Bacillus subtilis, Vibrio cholerae, Salmonella typhi, Salmonella typhimurium, Neisseria lactamica, Neisseria cinerea, Mycobacteria (e.g. M. tuberculosis), yeasts, etc.

The invention provides a process for producing a polypeptide of the invention, comprising the step of synthesising at least part of the polypeptide by chemical means.

### Nucleic Acids

The invention also provides nucleic acid encoding polypeptides and hybrid polypeptides of the invention. It also provides nucleic acid comprising a nucleotide sequence that encodes one or more polypeptides or hybrid polypeptides of the invention.

The invention also provides nucleic acid comprising nucleotide sequences having sequence identity to such nucleotide sequences. Identity between sequences is preferably determined by the Smith-Waterman homology search algorithm as described above. Such nucleic acids include those using alternative codons to encode the same amino acid.

The invention also provides nucleic acid which can hybridize to these nucleic acids. Hybridization reactions can be performed under conditions of different “stringency”. Conditions that increase stringency of a hybridization reaction of widely known and published in the art (e.g. page 7.52 of reference 214). Examples of relevant conditions include (in order of increasing stringency): incubation temperatures of 25° C., 37° C., 50° C., 55° C. and 68° C.; buffer concentrations of 10×SSC, 6×SSC, 1×SSC, 0.1×SSC (where SSC is 0.15 M NaCl and 15 mM citrate buffer) and their equivalents using other buffer systems; formamide concentrations of 0%, 25%, 50%, and 75%; incubation times from 5 minutes to 24 hours; 1, 2, or more washing steps; wash incubation times of 1, 2, or 15 minutes; and wash solutions of 6×SSC, 1×SSC, 0.1×SSC, or de-ionized water. Hybridization techniques and their optimization are well known in the art (e.g. see refs 22, 23, 214, 216, etc.).

In some embodiments, nucleic acid of the invention hybridizes to a target under low stringency conditions; in other embodiments it hybridizes under intermediate stringency conditions; in preferred embodiments, it hybridizes under high stringency conditions. An exemplary set of low stringency hybridization conditions is 50° C. and 10×SSC. An exemplary set of intermediate stringency hybridization conditions is 55° C. and 1×SSC. An exemplary set of high stringency hybridization conditions is 68° C. and 0.1×SSC.

The invention includes nucleic acid comprising sequences complementary to these sequences (e.g. for antisense or probing, or for use as primers).

Nucleic acids of the invention can be used in hybridisation reactions (e.g. Northern or Southern blots, or in nucleic acid microarrays or ‘gene chips’) and amplification reactions (e.g. PCR, SDA, SSSR, LCR, TMA, NASBA, etc.) and other nucleic acid techniques.

Nucleic acid according to the invention can take various forms (e.g. single-stranded, double-stranded, vectors, primers, probes, labelled etc.). Nucleic acids of the invention may be circular or branched, but will generally be linear. Unless otherwise specified or required, any embodiment of the invention that utilizes a nucleic acid may utilize both the double-stranded form and each of two complementary single-stranded forms which make up the double-stranded form. Primers and probes are generally single-stranded, as are antisense nucleic acids.

Nucleic acids of the invention are preferably provided in purified or substantially purified form i.e. substantially free from other nucleic acids (e.g. free from naturally-occurring nucleic acids), particularly from other pathogen of interest or host cell nucleic acids, generally being at least about 50% pure (by weight), and usually at least about 90% pure.

Nucleic acids of the invention may be prepared in many ways e.g. by chemical synthesis (e.g. phosphoramidite synthesis of DNA) in whole or in part, by digesting longer nucleic acids using nucleases (e.g. restriction enzymes), by joining shorter nucleic acids or nucleotides (e.g. using ligases or polymerases), from genomic or cDNA libraries, etc.

Nucleic acid of the invention may be attached to a solid support (e.g. a bead, plate, filter, film, slide, microarray support, resin, etc.). Nucleic acid of the invention may be labelled e.g. with a radioactive or fluorescent label, or a biotin label. This is particularly useful where the nucleic acid is to be used in detection techniques e.g. where the nucleic acid is a primer or as a probe.

The term “nucleic acid” includes in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus the invention includes mRNA, tRNA, rRNA, ribozymes, DNA, cDNA, recombinant nucleic acids, branched nucleic acids, plasmids, vectors, probes, primers, etc. Where nucleic acid of the invention takes the form of RNA, it may or may not have a 5′ cap.

Nucleic acids of the invention may be part of a vector i.e. part of a nucleic acid construct designed for transduction/transfection of one or more cell types. Vectors may be, for example, “cloning vectors” which are designed for isolation, propagation and replication of inserted nucleotides, “expression vectors” which are designed for expression of a nucleotide sequence in a host cell, “viral vectors” which is designed to result in the production of a recombinant virus or virus-like particle, or “shuttle vectors”, which comprise the attributes of more than one type of vector. Preferred vectors are plasmids, as mentioned above. A “host cell” includes an individual cell or cell culture which can be or has been a recipient of exogenous nucleic acid. Host cells include progeny of a single host cell, and the progeny may not necessarily be completely identical (in morphology or in total DNA complement) to the original parent cell due to natural, accidental, or deliberate mutation and/or change. Host cells include cells transfected or infected in vivo or in vitro with nucleic acid of the invention.

Where a nucleic acid is DNA, it will be appreciated that “U” in a RNA sequence will be replaced by “T” in the DNA. Similarly, where a nucleic acid is RNA, it will be appreciated that “T” in a DNA sequence will be replaced by “U” in the RNA.

The term “complement” or “complementary” when used in relation to nucleic acids refers to Watson-Crick base pairing. Thus the complement of C is G, the complement of G is C, the complement of A is T (or U), and the complement of T (or U) is A. It is also possible to use bases such as I (the purine inosine) e.g. to complement pyrimidines (C or T).

Nucleic acids of the invention can be used, for example: to produce polypeptides; as hybridization probes for the detection of nucleic acid in biological samples; to generate additional copies of the nucleic acids; to generate ribozymes or antisense oligonucleotides; as single-stranded DNA primers or probes; or as triple-strand forming oligonucleotides.

The invention provides a process for producing nucleic acid of the invention, wherein the nucleic acid is synthesised in part or in whole using chemical means.

The invention provides vectors comprising nucleotide sequences of the invention (e.g. cloning or expression vectors) and host cells transformed with such vectors.

Nucleic acid amplification according to the invention may be quantitative and/or real-time.

For certain embodiments of the invention, nucleic acids are preferably at least 7 nucleotides in length (e.g. 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 90, 100, 110, 120, 130, 140, 150, 160, 170, 180, 190, 200, 225, 250, 275, 300 nucleotides or longer).

For certain embodiments of the invention, nucleic acids are preferably at most 500 nucleotides in length (e.g. 450, 400, 350, 300, 250, 200, 150, 140, 130, 120, 110, 100, 90, 80, 75, 70, 65, 60, 55, 50, 45, 40, 39, 38, 37, 36, 35, 34, 33, 32, 31, 30, 29, 28, 27, 26, 25, 24, 23, 22, 21, 20, 19, 18, 17, 16, 15 nucleotides or shorter).

Primers and probes of the invention, and other nucleic acids used for hybridization, are preferably between 10 and 30 nucleotides in length (e.g. 10, 11, 12, 13, 14, 15, 16, 17, 18, 19, 20, 21, 22, 23, 24, 25, 26, 27, 28, 29, or 30 nucleotides).

### Immunogenic Compositions and Medicaments

Polypeptides of the invention are useful as active ingredients (immunogens) in immunogenic compositions, and such compositions may be useful as vaccines. Vaccines according to the invention may either be prophylactic (i.e. to prevent infection) or therapeutic (i.e. to treat infection), but will typically be prophylactic.

Immunogenic compositions will be pharmaceutically acceptable. They will usually include components in addition to the antigens e.g. they typically include one or more pharmaceutical carrier(s), excipient(s) and/or adjuvant(s). A thorough discussion of carriers and excipients is available in ref.211. Thorough discussions of vaccine adjuvants are available in refs. 24 and 25.

Compositions will generally be administered to a mammal in aqueous form. Prior to administration, however, the composition may have been in a non-aqueous form. For instance, although some vaccines are manufactured in aqueous form, then filled and distributed and administered also in aqueous form, other vaccines are lyophilised during manufacture and are reconstituted into an aqueous form at the time of use. Thus a composition of the invention may be dried, such as a lyophilised formulation.

The composition may include preservatives such as thiomersal or 2-phenoxyethanol. It is preferred, however, that the vaccine should be substantially free from (i.e. less than 5 μg/ml) mercurial material e.g. thiomersal-free. Vaccines containing no mercury are more preferred. Preservative-free vaccines are particularly preferred.

To improve thermal stability, a composition may include a temperature protective agent.

To control tonicity, it is preferred to include a physiological salt, such as a sodium salt. Sodium chloride (NaCl) is preferred, which may be present at between 1 and 20 mg/ml e.g. about 10±2 mg/ml NaCl. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, >0 preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer (particularly with an aluminum hydroxide adjuvant); or a citrate buffer. Buffers will typically be included in the 5-20 mM range.

The pH of a composition will generally be between 5.0 and 8.1, and more typically between 6.0 and 8.0 e.g. 6.5 and 7.5, or between 7.0 and 7.8.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Human vaccines are typically administered in a dosage volume of about 0.5 ml, although a half dose (i.e. about 0.25 ml) may be administered to children.

Immunogenic compositions of the invention may also comprise one or more immunoregulatory agents. Preferably, one or more of the immunoregulatory agents include one or more adjuvants. The adjuvants may include a TH1 adjuvant and/or a TH2 adjuvant, further discussed below.

Adjuvants which may be used in compositions of the invention include, but are not limited to:

### A. Mineral-Containing Compositions

Mineral containing compositions suitable for use as adjuvants in the invention include mineral salts, such as aluminium salts and calcium salts (or mixtures thereof). Calcium salts include calcium phosphate (e.g. the “CAP” particles disclosed in ref. 26). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt (27).

The adjuvants known as aluminum hydroxide and aluminum phosphate may be used. These names are conventional, but are used for convenience only, as neither is a precise description of the actual chemical compound which is present (e.g. see chapter 9 of reference 24). The invention can use any of the “hydroxide” or “phosphate” adjuvants that are in general use as adjuvants. The adjuvants known as “aluminium hydroxide” are typically aluminium oxyhydroxide salts, which are usually at least partially crystalline. The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt.

A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

Aluminium phosphate adjuvants generally have a PO₄/Al molar ratio between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml. A maximum of 0.85 mg/dose is preferred.

### B. Oil Emulsions

Oil emulsion compositions suitable for use as adjuvants in the invention include squalene-water emulsions, such as MF59 (Chapter 10 of ref. 24; see also ref. 28) (5% Squalene, 0.5% Tween 80, and 0.5% Span 85, formulated into submicron particles using a microfluidizer). Complete Freund's adjuvant (CFA) and incomplete Freund's adjuvant (IFA) may also be used.

Various oil-in-water emulsion adjuvants are known, and they typically include at least one oil and at least one surfactant, with the oil(s) and surfactant(s) being biodegradable (metabolisable) and biocompatible. The oil droplets in the emulsion are generally less than 5 μm in diameter, and ideally have a sub-micron diameter, with these small sizes being achieved with a microfluidiser to provide stable emulsions. Droplets with a size less than 220 nm are preferred as they can be subjected to filter sterilization.

The emulsion can comprise oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Preferred emulsion adjuvants have an average droplets size of <1 μm e.g. <750 nm, <500 nm, <400 nm, <300 nm, <250 nm, <220 nm, <200 nm, or smaller. These droplet sizes can conveniently be achieved by techniques such as microfluidisation.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ (29-31), as described in more detail in Chapter 10 of ref. 32 and chapter 12 of ref. 33. The MF59 emulsion advantageously includes citrate ions e.g. 10 mM sodium citrate buffer.
An emulsion of squalene, a tocopherol, and Tween 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. Squalene and Tween 80 may be present volume ratio of about 5:2. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 110 μg/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant (34) (0.05-1% Thr-MDP, 5% squalane, 2.5% Pluronic L121 and 0.2% polysorbate 80). It can also be used without the Thr-MDP, as in the “AF” adjuvant (35) (5% squalane, 1.25% Pluronic L121 and 0.2% polysorbate 80). Microfluidisation is preferred.
An emulsion comprising squalene, an aqueous solvent, a polyoxyethylene alkyl ether hydrophilic nonionic surfactant (e.g. polyoxyethylene (12) cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm (36). The emulsion may also include one or more of: alditol; a cryoprotective agent (e.g. a sugar, such as dodecylmaltoside and/or sucrose); and/or an alkylpolyglycoside. Such emulsions may be lyophilized.
An emulsion of squalene, poloxamer 105 and Abil-Care (37). The final concentration (weight) of these components in adjuvanted vaccines are 5% squalene, 4% poloxamer 105 (pluronic polyol) and 2% Abil-Care 85 (Bis-PEG/PPG-16/16 PEG/PPG-16/16 dimethicone; caprylic/capric triglyceride).
An emulsion having from 0.5-50% of an oil, 0.1-10% of a phospholipid, and 0.05-5% of a non-ionic surfactant. As described in reference 38, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 39, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles (40).
An emulsion comprising a mineral oil, a non-ionic lipophilic ethoxylated fatty alcohol, and a non-ionic hydrophilic surfactant (e.g. an ethoxylated fatty alcohol and/or polyoxyethylene-polyoxypropylene block copolymer) (41).
An emulsion comprising a mineral oil, a non-ionic hydrophilic ethoxylated fatty alcohol, and a non-ionic lipophilic surfactant (e.g. an ethoxylated fatty alcohol and/or polyoxyethylene-polyoxypropylene block copolymer) (41).

In some embodiments an emulsion may be mixed with antigen extemporaneously, at the time of delivery, and thus the adjuvant and antigen may be kept separately in a packaged or distributed vaccine, ready for final formulation at the time of use. In other embodiments an emulsion is mixed with antigen during manufacture, and thus the composition is packaged in a liquid adjuvanted form. The antigen will generally be in an aqueous form, such that the vaccine is finally prepared by mixing two liquids. The volume ratio of the two liquids for mixing can vary (e.g. between 5:1 and 1:5) but is generally about 1:1. Where concentrations of components are given in the above descriptions of specific emulsions, these concentrations are typically for an undiluted composition, and the concentration after mixing with an antigen solution will thus decrease.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group (42). They also have antioxidant properties that may help to stabilize the emulsions (43). A preferred α-tocopherol is DL-α-tocopherol, and the preferred salt of this tocopherol is the succinate. The succinate salt has been found to cooperate with TNF-related ligands in vivo.

### C. Saponin Formulations (Chapter 22 of ref 24)

Saponin formulations may also be used as adjuvants in the invention. Saponins are a heterogeneous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 44. Saponin formulations may also comprise a sterol, such as cholesterol (45).

Combinations of saponins and cholesterols can be used to form unique particles called immunostimulating complexs (ISCOMs) (chapter 23 of ref. 24). ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 45-47. Optionally, the ISCOMS may be devoid of additional detergent (48).

A review of the development of saponin based adjuvants can be found in refs. 49 & 50.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in refs. 51-56. Virosomes are discussed further in, for example, ref. 57

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred “small particle” form of 3 De-O-acylated monophosphoryl lipid A is disclosed in ref. 58. Such “small particles” of 3dMPL are small enough to be sterile filtered through a 0.22 μm membrane (58). Other non-toxic LPS derivatives include monophosphoryl lipid A mimics, such as aminoalkyl glucosaminide phosphate derivatives e.g. RC-529 (59,60).

Lipid A derivatives include derivatives of lipid A from Escherichia coli such as OM-174. OM-174 is described for example in refs. 61 & 62.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory.

The CpG's can include nucleotide modifications/analogs such as phosphorothioate modifications and can be double-stranded or single-stranded. References 63, 64 and 65 disclose possible analog substitutions e.g. replacement of guanosine with 2′-deoxy-7-deazaguanosine. The adjuvant effect of CpG oligonucleotides is further discussed in refs. 66-71.

The CpG sequence may be directed to TLR9, such as the motif GTCGTT or TTCGTT (72). The CpG sequence may be specific for inducing a Th1 immune response, such as a CpG-A ODN, or it may be more specific for inducing a B cell response, such a CpG-B ODN. CpG-A and CpG-B ODNs are discussed in refs. 73-75. Preferably, the CpG is a CpG-A ODN.

Preferably, the CpG oligonucleotide is constructed so that the 5′ end is accessible for receptor recognition. Optionally, two CpG oligonucleotide sequences may be attached at their 3′ ends to form “immunomers”. See, for example, refs. 72 & 76-78.

A useful CpG adjuvant is CpG7909, also known as ProMune™ (Coley Pharmaceutical Group, Inc.). Another is CpG1826. As an alternative, or in addition, to using CpG sequences, TpG sequences can be used (79), and these oligonucleotides may be free from unmethylated CpG motifs. The immunostimulatory oligonucleotide may be pyrimidine-rich. For example, it may comprise more than one consecutive thymidine nucleotide (e.g. TTTT, as disclosed in ref. 79), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref 79), and/or it may have a nucleotide composition with >25% cytosine (e.g. >35%, >40%, >50%, >60%, >80%, etc.). These oligonucleotides may be free from unmethylated CpG motifs. Immunostimulatory oligonucleotides will typically comprise at least 20 nucleotides. They may comprise fewer than 100 nucleotides.

A particularly useful adjuvant based around immunostimulatory oligonucleotides is known as IC-31™ (80). Thus an adjuvant used with the invention may comprise a mixture of (i) an oligonucleotide (e.g. between 15-40 nucleotides) including at least one (and preferably multiple) CpI motifs (i.e. a cytosine linked to an inosine to form a dinucleotide), and (ii) a polycationic polymer, such as an oligopeptide (e.g. between 5-20 amino acids) including at least one (and preferably multiple) Lys-Arg-Lys tripeptide sequence(s). The oligonucleotide may be a deoxynucleotide comprising 26-mer sequence 5′-(IC)₁₃-3′ (SEQ ID NO: 96). The polycationic polymer may be a peptide comprising 11-mer amino acid sequence KLKLLLLLKLK (SEQ ID NO: 97).

Bacterial ADP-ribosylating toxins and detoxified derivatives thereof may be used as adjuvants in the invention. Preferably, the protein is derived from E. coli (E. coli heat labile enterotoxin “LT”), cholera (“CT”), or pertussis (“PT”). The use of detoxified ADP-ribosylating toxins as mucosal adjuvants is described in ref. 81 and as parenteral adjuvants in ref. 82. The toxin or toxoid is preferably in the form of a holotoxin, comprising both A and B subunits. Preferably, the A subunit contains a detoxifying mutation; preferably the B subunit is not mutated. Preferably, the adjuvant is a detoxified LT mutant such as LT-K63, LT-R72, and LT-G192. The use of ADP-ribosylating toxins and detoxified derivatives thereof, particularly LT-K63 and LT-R72, as adjuvants can be found in refs. 83-90. A useful CT mutant is or CT-E29H (91). Numerical reference for amino acid substitutions is preferably based on the alignments of the A and B subunits of ADP-ribosylating toxins set forth in ref. 92, specifically incorporated herein by reference in its entirety.

### F. Human Immunomodulators

Human immunomodulators suitable for use as adjuvants in the invention include cytokines, such as interleukins (e.g. IL-1, IL-2, IL-4, IL-5, IL-6, IL-7, IL-12 (93), etc.) (94), interferons (e.g. interferon-γ), macrophage colony stimulating factor, and tumor necrosis factor. A preferred immunomodulator is IL-12.

### G. Bioadhesives and Mucoadhesives

Bioadhesives and mucoadhesives may also be used as adjuvants in the invention. Suitable bioadhesives include esterified hyaluronic acid microspheres (95) or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention (96).

### H. Microparticles

Microparticles may also be used as adjuvants in the invention. Microparticles (i.e. a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes (Chapters 13 & 14 of ref 24)

Examples of liposome formulations suitable for use as adjuvants are described in refs. 97-99.

### J. Polyoxyethylene Ether and Polyoxyethylene Ester Formulations

Adjuvants suitable for use in the invention include polyoxyethylene ethers and polyoxyethylene esters (100). Such formulations further include polyoxyethylene sorbitan ester surfactants in combination with an octoxynol (101) as well as polyoxyethylene alkyl ethers or ester surfactants in combination with at least one additional non-ionic surfactant such as an octoxynol (102). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

### K. Phosphazenes

A phosphazene, such as poly(di(carboxylatophenoxy)phosphazene) (“PCPP”) as described, for example, in references 103 and 104, may be used.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine MTP-PE).

### M. Imidazoquinolone Compounds.

Examples of imidazoquinolone compounds suitable for use adjuvants in the invention include Imiquimod (“R-837”) (105,106), Resiquimod (“R-848”) (107), and their analogs; and salts thereof (e.g. the hydrochloride salts). Further details about immunostimulatory imidazoquinolines can be found in references 108 to 112.

### N. Substituted Ureas

Substituted ureas useful as adjuvants include compounds of formula I, II or III, or salts thereof:

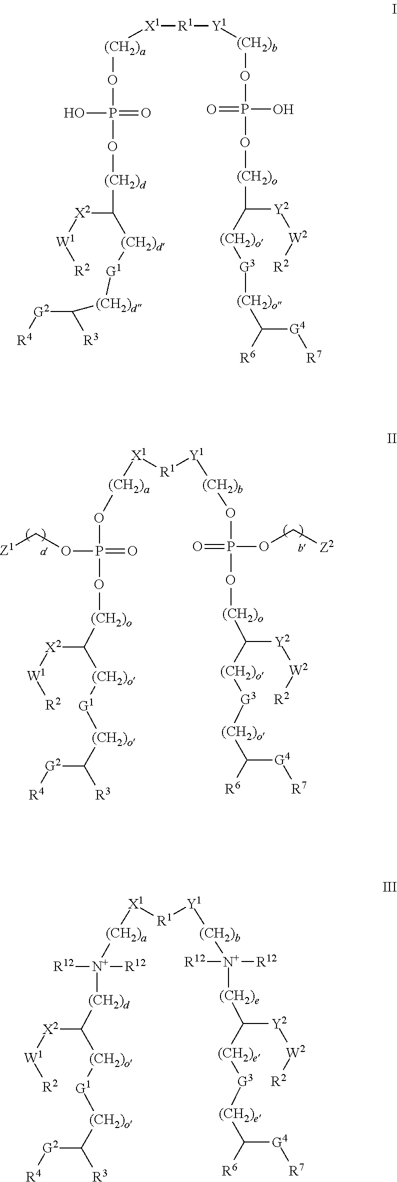

as defined in reference 113, such as ‘ER 803058’, ‘ER 803732’, ‘ER 804053’, ER 804058′, ‘ER 804059’, ‘ER 804442’, ‘ER 804680’, ‘ER 804764’, ER 803022 or ‘ER 804057’ e.g.:

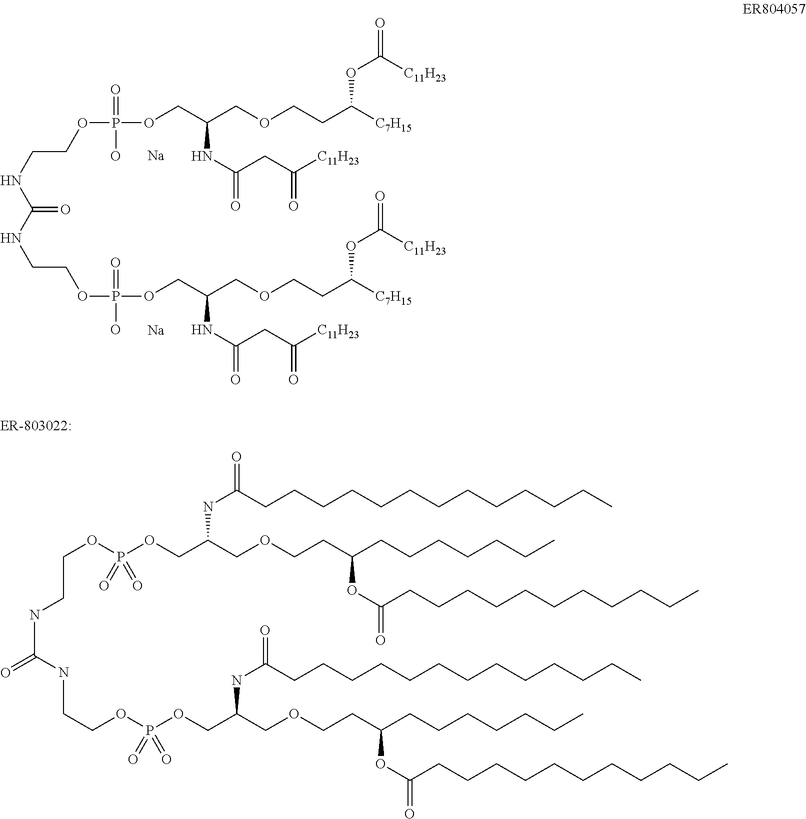

### O. Further Adjuvants

Further adjuvants that may be used with the invention include:
An aminoalkyl glucosaminide phosphate derivative, such as RC-529 (114,115).
A thiosemicarbazone compound, such as those disclosed in reference 116. Methods of formulating, manufacturing, and screening for active compounds are also described in reference 116. The thiosemicarbazones are particularly effective in the stimulation of human peripheral blood mononuclear cells for the production of cytokines, such as TNF-α.
tryptanthrin compound, such as those disclosed in reference 117. Methods of formulating, manufacturing, and screening for active compounds are also described in reference 117. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

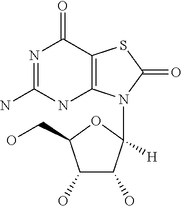

and prodrugs thereof; (b) ANA975; (c) ANA-025-1; (d) ANA380; (e) the compounds disclosed in references 118 to 120
Loxoribine (7-allyl-8-oxoguanosine) (121).
Compounds disclosed in reference 122, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds (123,124), Hydrapthalamide compounds, Benzophenone compounds, Isoxazole compounds, Sterol compounds, Quinazilinone compounds, Pyrrole compounds (125), Anthraquinone compounds, Quinoxaline compounds, Triazine compounds, Pyrazalopyrimidine compounds, and Benzazole compounds (126).
Compounds containing lipids linked to a phosphate-containing acyclic backbone, such as the TLR4 antagonist E5564 (127,128):
A polyoxidonium polymer (129,130) or other N-oxidized polyethylene-piperazine derivative.
Methyl inosine 5′-monophosphate (“MIMP”) (131).
A polyhydroxlated pyrrolizidine compound (132), such as one having formula:

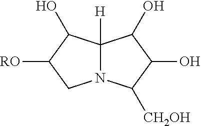

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A CD1d ligand, such as an α-glycosylceramide (133-140) (e.g. α-galactosylceramide), phytosphingosine-containing α-glycosylceramides, OCH, KRN7000 ((2S,3S,4R)-1-O-(α-D-galactopyranosyl)-2-(N-hexacosanoylamino)-1,3,4-octadecanetriol), CRONY-101, 3″-O-sulfo-galactosylceramide, etc.
A gamma inulin (141) or derivative thereof, such as algammulin.

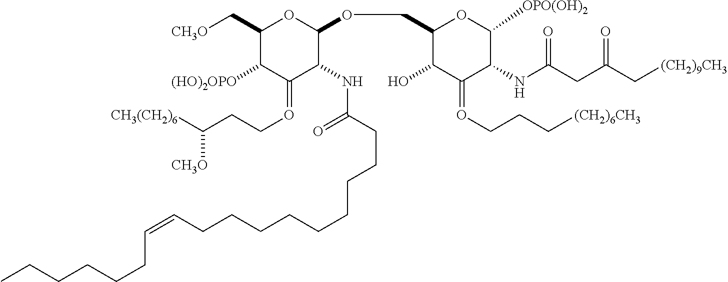

### Adjuvant Combinations

The invention may also comprise combinations of aspects of one or more of the adjuvants identified above. For example, the following adjuvant compositions may be used in the invention: (1) a saponin and an oil-in-water emulsion (142); (2) a saponin (e.g. QS21)+a non-toxic LPS derivative (e.g. 3dMPL) (143); (3) a saponin (e.g. QS21)+a non-toxic LPS derivative (e.g. 3dMPL)+a cholesterol; (4) a saponin (e.g. QS21)+3dMPL+IL-12 (optionally+a sterol) (144); (5) combinations of 3dMPL with, for example, QS21 and/or oil-in-water emulsions (145); (6) SAF, containing 10% squalane, 0.4% Tween 80™, 5% pluronic-block polymer L121, and thr-MDP, either microfluidized into a submicron emulsion or vortexed to generate a larger particle size emulsion. (7) Ribi™ adjuvant system (RAS), (Ribi Immunochem) containing 2% squalene, 0.2% Tween 80, and one or more bacterial cell wall components from the group consisting of monophosphorylipid A (MPL), trehalose dimycolate (TDM), and cell wall skeleton (CWS), preferably MPL+CWS (Detox™); and (8) one or more mineral salts (such as an aluminum salt)+a non-toxic derivative of LPS (such as 3dMPL).

Other substances that act as immunostimulating agents are disclosed in chapter 7 of ref. 24.

The use of an aluminium hydroxide and/or aluminium phosphate adjuvant is particularly preferred, and antigens are generally adsorbed to these salts. Calcium phosphate is another preferred adjuvant. Other preferred adjuvant combinations include combinations of Th1 and Th2 adjuvants such as CpG & alum or resiquimod & alum. A combination of aluminium phosphate and 3dMPL may be used.

The compositions of the invention may elicit both a cell mediated immune response as well as a humoral immune response. This immune response will preferably induce long lasting (e.g. neutralising) antibodies and a cell mediated immunity that can quickly respond upon exposure to pnuemococcus.

Two types of T cells, CD4 and CD8 cells, are generally thought necessary to initiate and/or enhance cell mediated immunity and humoral immunity. CD8 T cells can express a CD8 co-receptor and are commonly referred to as Cytotoxic T lymphocytes (CTLs). CD8 T cells are able to recognized or interact with antigens displayed on MHC Class I molecules.

CD4 T cells can express a CD4 co-receptor and are commonly referred to as T helper cells. CD4 T cells are able to recognize antigenic peptides bound to MHC class II molecules. Upon interaction with a MHC class II molecule, the CD4 cells can secrete factors such as cytokines. These secreted cytokines can activate B cells, cytotoxic T cells, macrophages, and other cells that participate in an immune response. Helper T cells or CD4+ cells can be further divided into two functionally distinct subsets: TH1 phenotype and TH2 phenotypes which differ in their cytokine and effector function.

Activated TH1 cells enhance cellular immunity (including an increase in antigen-specific CTL production) and are therefore of particular value in responding to intracellular infections. Activated TH1 cells may secrete one or more of IL-2, IFN-γ, and TNF-β. A TH1 immune response may result in local inflammatory reactions by activating macrophages, NK (natural killer) cells, and CD8 cytotoxic T cells (CTLs). A TH1 immune response may also act to expand the immune response by stimulating growth of B and T cells with IL-12. TH1 stimulated B cells may secrete IgG2a.

Activated TH2 cells enhance antibody production and are therefore of value in responding to extracellular infections. Activated TH2 cells may secrete one or more of IL-4, IL-5, IL-6, and IL-10. A TH2 immune response may result in the production of IgG1, IgE, IgA and memory B cells for future protection.

An enhanced immune response may include one or more of an enhanced TH1 immune response and a TH2 immune response.

A TH1 immune response may include one or more of an increase in CTLs, an increase in one or more of the cytokines associated with a TH1 immune response (such as IL-2, IFN-γ, and TNF-β), an increase in activated macrophages, an increase in NK activity, or an increase in the production of IgG2a. Preferably, the enhanced TH1 immune response will include an increase in IgG2a production.

A TH1 immune response may be elicited using a TH1 adjuvant. A TH1 adjuvant will generally elicit increased levels of IgG2a production relative to immunization of the antigen without adjuvant. TH1 adjuvants suitable for use in the invention may include for example saponin formulations, virosomes and virus like particles, non-toxic derivatives of enterobacterial lipopolysaccharide (LPS), immunostimulatory oligonucleotides. Immunostimulatory oligonucleotides, such as oligonucleotides containing a CpG motif, are preferred TH1 adjuvants for use in the invention.

A TH2 immune response may include one or more of an increase in one or more of the cytokines associated with a TH2 immune response (such as IL-4, IL-5, IL-6 and IL-10), or an increase in the production of IgG1, IgE, IgA and memory B cells. Preferably, the enhanced TH2 immune response will include an increase in IgG1 production.

A TH2 immune response may be elicited using a TH2 adjuvant. A TH2 adjuvant will generally elicit increased levels of IgG1 production relative to immunization of the antigen without adjuvant. TH2 adjuvants suitable for use in the invention include, for example, mineral containing compositions, oil-emulsions, and ADP-ribosylating toxins and detoxified derivatives thereof. Mineral containing compositions, such as aluminium salts are preferred TH2 adjuvants for use in the invention.

Preferably, the invention includes a composition comprising a combination of a TH1 adjuvant and a TH2 adjuvant. Preferably, such a composition elicits an enhanced TH1 and an enhanced TH2 response, i.e., an increase in the production of both IgG1 and IgG2a production relative to immunization without an adjuvant. Still more preferably, the composition comprising a combination of a TH1 and a TH2 adjuvant elicits an increased TH1 and/or an increased TH2 immune response relative to immunization with a single adjuvant (i.e., relative to immunization with a TH1 adjuvant alone or immunization with a TH2 adjuvant alone).

The immune response may be one or both of a TH1 immune response and a TH2 response. Preferably, immune response provides for one or both of an enhanced TH1 response and an enhanced TH2 response.

The enhanced immune response may be one or both of a systemic and a mucosal immune response. Preferably, the immune response provides for one or both of an enhanced systemic and an enhanced mucosal immune response. Preferably the mucosal immune response is a TH2 immune response. Preferably, the mucosal immune response includes an increase in the production of IgA.

Pathogens expressing factor H binding proteins can cause disease at a number of anatomical locations and so the compositions of the invention may be prepared in various forms. For example, the compositions may be prepared as injectables, either as liquid solutions or suspensions. Solid forms suitable for solution in, or suspension in, liquid vehicles prior to injection can also be prepared (e.g. a lyophilised composition or a spray-freeze dried composition). The composition may be prepared for topical administration e.g. as an ointment, cream or powder. The composition may be prepared for oral administration e.g. as a tablet or capsule, as a spray, or as a syrup (optionally flavoured). The composition may be prepared for pulmonary administration e.g. as an inhaler, using a fine powder or a spray. The composition may be prepared as a suppository or pessary. The composition may be prepared for nasal, aural or ocular administration e.g. as drops. The composition may be in kit form, designed such that a combined composition is reconstituted just prior to administration to a patient. Such kits may comprise one or more antigens in liquid form and one or more lyophilised antigens.

Where a composition is to be prepared extemporaneously prior to use (e.g. where a component is presented in lyophilised form) and is presented as a kit, the kit may comprise two vials, or it may comprise one ready-filled syringe and one vial, with the contents of the syringe being used to reactivate the contents of the vial prior to injection.

Immunogenic compositions used as vaccines comprise an immunologically effective amount of antigen(s), as well as any other components, as needed. By ‘immunologically effective amount’, it is meant that the administration of that amount to an individual, either in a single dose or as part of a series, is effective for treatment or prevention. This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials.

### Methods of Treatment, and Administration of the Vaccine

The invention also provides a method for raising an immune response in a mammal comprising the step of administering an effective amount of a composition of the invention. The immune response is preferably protective and preferably involves antibodies and/or cell-mediated immunity. The method may raise a booster response.

The invention also provides a polypeptide of the invention for use as a medicament e.g. for use in raising an immune response in a mammal.

The invention also provides the use of a polypeptide of the invention in the manufacture of a medicament for raising an immune response in a mammal.

The invention also provides a delivery device pre-filled with an immunogenic composition of the invention.

By raising an immune response in the mammal by these uses and methods, the mammal can be protected against infection by pathogens expressing factor H binding proteins, including N. meningitidis strains of all serogroups and of serogroups A, B, C, W-135 and Y in particular. The mammal is preferably a human, but may be e.g. a cow, a pig, a chicken, a cat or a dog, as the pathogens covered herein may be problematic across a wide range of species. Where the vaccine is for prophylactic use, the human is preferably a child (e.g. a toddler or infant) or a teenager; where the vaccine is for therapeutic use, the human is preferably a teenager or an adult. A vaccine intended for children may also be administered to adults e.g. to assess safety, dosage, immunogenicity, etc.

One way of checking efficacy of therapeutic treatment involves monitoring E. coli infection after administration of the compositions of the invention. One way of checking efficacy of prophylactic treatment involves monitoring immune responses, systemically (such as monitoring the level of IgG1 and IgG2a production) and/or mucosally (such as monitoring the level of IgA production), against the antigens in the compositions of the invention after administration of the composition. Typically, antigen-specific serum antibody responses are determined post-immunisation but pre-challenge whereas antigen-specific mucosal antibody responses are determined post-immunisation and post-challenge.

Another way of assessing the immunogenicity of the compositions of the present invention is to express the proteins recombinantly for screening patient sera or mucosal secretions by immunoblot and/or microarrays. A positive reaction between the protein and the patient sample indicates that the patient has mounted an immune response to the protein in question. This method may also be used to identify immunodominant antigens and/or epitopes within antigens.

The efficacy of vaccine compositions can also be determined in vivo by challenging appropriate animal models of the pathogen of interest infection.

Compositions of the invention will generally be administered directly to a patient. Direct delivery may be accomplished by parenteral injection (e.g. subcutaneously, intraperitoneally, intravenously, intramuscularly, or to the interstitial space of a tissue), or mucosally, such as by rectal, oral (e.g. tablet, spray), vaginal, topical, transdermal or transcutaneous, intranasal, ocular, aural, pulmonary or other mucosal administration.

The invention may be used to elicit systemic and/or mucosal immunity, preferably to elicit an enhanced systemic and/or mucosal immunity.

Preferably the enhanced systemic and/or mucosal immunity is reflected in an enhanced TH1 and/or TH2 immune response. Preferably, the enhanced immune response includes an increase in the production of IgG1 and/or IgG2a and/or IgA.

Dosage can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines of the invention may be used to treat both children and adults. Thus a human patient may be less than 1 year old, 1-5 years old, 5-15 years old, 15-55 years old, or at least 55 years old. Preferred patients for receiving the vaccines are the elderly (e.g. ≧50 years old, ≧60 years old, and preferably ≧65 years), the young (e.g. ≦5 years old), hospitalised patients, healthcare workers, armed service and military personnel, pregnant women, the chronically ill, or n immunodeficient patients. The vaccines are not suitable solely for these groups, however, and may be used more generally in a population.

Vaccines of the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, etc.

### Nucleic Acid Immunisation

The immunogenic compositions described above include polypeptide antigens. In all cases, however, the polypeptide antigens can be replaced by nucleic acids (typically DNA) encoding those polypeptides, to give compositions, methods and uses based on nucleic acid immunisation. Nucleic acid immunisation is now a developed field (e.g. see references 146 to 153 etc.).

The nucleic acid encoding the immunogen is expressed in vivo after delivery to a patient and the expressed immunogen then stimulates the immune system. The active ingredient will typically take the form of a nucleic acid vector comprising: (i) a promoter; (ii) a sequence encoding the immunogen, operably linked to the promoter; and optionally (iii) a selectable marker. Preferred vectors may further comprise (iv) an origin of replication; and (v) a transcription terminator downstream of and operably linked to (ii). In general, (i) & (v) will be eukaryotic and (iii) & (iv) will be prokaryotic.

Preferred promoters are viral promoters e.g. from cytomegalovirus (CMV). The vector may also include transcriptional regulatory sequences (e.g. enhancers) in addition to the promoter and which interact functionally with the promoter. Preferred vectors include the immediate-early CMV enhancer/promoter, and more preferred vectors also include CMV intron A. The promoter is operably linked to a downstream sequence encoding an immunogen, such that expression of the immunogen-encoding sequence is under the promoter's control.

Where a marker is used, it preferably functions in a microbial host (e.g. in a prokaryote, in a bacteria, in a yeast). The marker is preferably a prokaryotic selectable marker (e.g. transcribed under the control of a prokaryotic promoter). For convenience, typical markers are antibiotic resistance genes.

The vector of the invention is preferably an autonomously replicating episomal or extrachromosomal vector, such as a plasmid.

The vector of the invention preferably comprises an origin of replication. It is preferred that the origin of replication is active in prokaryotes but not in eukaryotes.

Preferred vectors thus include a prokaryotic marker for selection of the vector, a prokaryotic origin of replication, but a eukaryotic promoter for driving transcription of the immunogen-encoding sequence. The vectors will therefore (a) be amplified and selected in prokaryotic hosts without polypeptide expression, but (b) be expressed in eukaryotic hosts without being amplified. This arrangement is ideal for nucleic acid immunization vectors.

The vector of the invention may comprise a eukaryotic transcriptional terminator sequence downstream of the coding sequence. This can enhance transcription levels. Where the coding sequence does not have its own, the vector of the invention preferably comprises a polyadenylation sequence. A preferred polyadenylation sequence is from bovine growth hormone.

The vector of the invention may comprise a multiple cloning site.

In addition to sequences encoding the immunogen and a marker, the vector may comprise a second eukaryotic coding sequence. The vector may also comprise an IRES upstream of said second sequence in order to permit translation of a second eukaryotic polypeptide from the same transcript as the immunogen. Alternatively, the immunogen-coding sequence may be downstream of an IRES.

The vector of the invention may comprise unmethylated CpG motifs e.g. unmethylated DNA sequences which have in common a cytosine preceding a guanosine, flanked by two 5′ purines and two 3′ pyrimidines. In their unmethylated form these DNA motifs have been demonstrated to be potent stimulators of several types of immune cell.

Vectors may be delivered in a targeted way. Receptor-mediated DNA delivery techniques are described in, for example, references 154 to 159. Therapeutic compositions containing a nucleic acid are administered in a range of about 100 ng to about 200 mg of DNA for local administration in a gene therapy protocol. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action (e.g. for enhancing or inhibiting levels of the encoded gene product) and efficacy of transformation and expression are considerations which will affect the dosage required for ultimate efficacy. Where greater expression is desired over a larger area of tissue, larger amounts of vector or the same amounts re-administered in a successive protocol of administrations, or several administrations to different adjacent or close tissue portions may be required to effect a positive therapeutic outcome. In all cases, routine experimentation in clinical trials will determine specific ranges for optimal therapeutic effect.

Vectors can be delivered using gene delivery vehicles. The gene delivery vehicle can be of viral or non-viral origin (see generally references 160 to 163).

Viral-based vectors for delivery of a desired nucleic acid and expression in a desired cell are well known in the art. Exemplary viral-based vehicles include, but are not limited to, recombinant retroviruses (e.g. references 164 to 174), alphavirus-based vectors (e.g. Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532); hybrids or chimeras of these viruses may also be used), poxvirus vectors (e.g. vaccinia, fowlpox, canarypox, modified vaccinia Ankara, etc.), adenovirus vectors, and adeno-associated virus (AAV) vectors (e.g. see refs. 175 to 180). Administration of DNA linked to killed adenovirus (181) can also be employed.

Non-viral delivery vehicles and methods can also be employed, including, but not limited to, polycationic condensed DNA linked or unlinked to killed adenovirus alone (e.g. 181), ligand-linked DNA (182), eukaryotic cell delivery vehicles cells (e.g. refs. 183 to 187) and nucleic charge neutralization or fusion with cell membranes. Naked DNA can also be employed.

Exemplary naked DNA introduction methods are described in refs. 188 and 189. Liposomes (e.g. immunoliposomes) that can act as gene delivery vehicles are described in refs. 190 to 194. Additional approaches are described in references 195 & 196.

Further non-viral delivery suitable for use includes mechanical delivery systems such as the approach described in ref. 196. Moreover, the coding sequence and the product of expression of such can be delivered through deposition of photopolymerized hydrogel materials or use of ionizing radiation (e.g. refs. 197 & 198). Other conventional methods for gene delivery that can be used for delivery of the coding sequence include, for example, use of hand-held gene transfer particle gun (199) or use of ionizing radiation for activating transferred genes (197 & 198).

Delivery DNA using PLG {poly(lactide-co-glycolide)} microparticles is a particularly preferred method e.g. by adsorption to the microparticles, which are optionally treated to have a negatively-charged surface (e.g. treated with SDS) or a positively-charged surface (e.g. treated with a cationic detergent, such as CTAB).

### Disclaimers

In some embodiments, the invention may not encompass the use of multiple factor H binding polypeptides which are NMB1870, NMB2091, and NMB1030 (or two of the foregoing). Such polypeptide combinations are disclosed in at least WO04/032958 for use in immunising against Neisserial infections.

In other embodiments, however, the polypeptide combinations of WO04/032958 are encompassed, but e.g. for new medical purposes or in further combinations. For example, as disclosed herein, NMB0667 has also been demonstrated to be a factor H binding protein and therefore may be used in further combination with the polypeptide combinations of WO04/032958.

In some embodiments, the invention may not encompass the use of multiple factor H binding polypeptides which are homologs within related strains. By way of example, use of multiple factor H binding polypeptides which are NMB 1870s from related Neisserial strains are disclosed in at least WO2004/048404 for use in immunising against Neisserial infections. By way of further example, use of multiple factor H binding polypeptides which are M proteins from related strains are disclosed in at least WO2003/065973 for use in immunising against Neisserial infections.

In other embodiments, however, the polypeptide combinations of WO2004/048404 and WO2003/065973 are encompassed, but e.g. for new medical purposes or in further combinations.

### Antibodies

Antibodies against factor H binding proteins can be used for passive immunisation (200). In certain embodiments, the compositions would include antibodies against at least two different factor H binding proteins from the pathogenic organism of interest or from a Neisserial strain (e.g., antibodies to NMB1870, NMB2091, NMB1030, NMB0667, or Por1A), an Actinobacillus strain (e.g., antibodies to Omp100), a Borrelia strain (e.g., antibodies to CRASPS, ERP, FHBP19/FhbA, and FHBP28), a Leptospira strain (e.g., antibodies to LfhA), a Pseudomonas strain (e.g., Tuf), a Streptococcus strain (e.g., antibodies to Bac, Fba, Hic, M protein, PspC, or Se18.9), a Yersinia strain (e.g., antibodies to YadA), or a Candida strain (e.g., antibodies to Gpm1p). Thus the invention provides an antibody that binds to a polypeptide selected from SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107.

The invention also provides the use of such antibodies or compositions in therapy. The invention also provides the use of such antibodies or compositions in the manufacture of a medicament. The invention also provides a method for treating a mammal comprising the step of administering an effective amount of an antibody or composition of the invention. As described above for immunogenic compositions, these methods and uses allow a mammal to be protected against infection by the pathogen of interest or against a Neisserial strain, an Actinobacillus strain, a Borrelia strain, a Leptospira strain, a Pseudomonas strain, a Streptococcus strain, a Yersinia strain, or a Candida strain.

The term “antibody” includes intact immunoglobulin molecules, as well as fragments thereof which are capable of binding an antigen. These include hybrid (chimeric) antibody molecules (201, 202); F(ab′)2 and F(ab) fragments and Fv molecules; non-covalent heterodimers (203, 204); single-chain Fv molecules (sFv) (205); dimeric and trimeric antibody fragment constructs; minibodies (206, 207); humanized antibody molecules (208-210); and any functional fragments obtained from such molecules, as well as antibodies obtained through non-conventional processes such as phage display. Preferably, the antibodies are monoclonal antibodies. Methods of obtaining monoclonal antibodies are well known in the art. Humanised or fully-human antibodies are preferred.

### General

The practice of the present invention will employ, unless otherwise indicated, conventional methods of chemistry, biochemistry, molecular biology, immunology and pharmacology, within the skill of the art. Such techniques are explained fully in the literature. See, e.g., references 211-218, etc.

The term “comprising” encompasses “including” as well as “consisting” e.g. a composition “comprising” X may consist exclusively of X or may include something additional e.g. X+Y.

The term “about” in relation to a numerical value x means, for example, x+10%.

“GI” numbering is used herein. A GI number, or “GenInfo Identifier”, is a series of digits assigned consecutively to each sequence record processed by NCBI when sequences are added to its databases. The GI number bears no resemblance to the accession number of the sequence record. When a sequence is updated (e.g. for correction, or to add more annotation or information) then it receives a new GI number. Thus the sequence associated with a given GI number is never changed.

References to a percentage sequence identity between two amino acid sequences means that, when aligned, that percentage of amino acids are the same in comparing the two sequences. This alignment and the percent homology or sequence identity can be determined using software programs known in the art, for example those described in section 7.7.18 of ref. 219. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref. 220.

### BRIEF DESCRIPTION OF DRAWINGS

FIG. 1 shows binding of factor H to various Neisseria antigens. Each well of the microtiter plate was coated with 1 ug of the applicable antigens. Binding was assayed in a total volume of 100 μl/well with either 1 μg/ml (white bars) or 10 μg/ml (grey bars) of factor H.

FIG. 2 shows the dose response of factor H binding to 1 ug/well of the different antigens. Factor H binding was tested at four concentrations of factor H: 0.01 μg/ml, 0.1 μg/ml, 1 μg/ml, and 10 μg/ml.

FIG. 3 shows the time-course of factor H binding to 1 ug/well of NMB1030. The time course of binding was assayed at two concentrations of factor H: 1 μg/ml, and 10 μg/ml. Factor H binding was assayed at each concentration at 30, 60, 90, and 120 minutes.

FIGS. 4 and 5 show the effect of competitive binding between PTX3 (the native binding partner for factor H) and different Neisserial antigens for factor H using two different concentrations of PTX3.

### BRIEF DESCRIPTION OF SEQUENCE LISTING

| SEQ ID | Description |
|---|---|
| 1 | NMB1870 |
| 2 | Nucleic acid sequence encoding SEQ ID NO: 1 |
| 3 | NMA0586-ortholog of NMB1870 and having identity = 0.957 to NMB1870 |
| 4 | Nucleic acid sequence encoding SEQ ID NO: 3 |
| 5 | NMCC_0351-ortholog of NMB1870 and having identity = 0.939 to NMB1870 |
| 6 | Nucleic acid sequence encoding SEQ ID NO: 5 |
| 7 | NMC0349-ortholog of NMB1870 and having identity = 0.714 to NMB1870 |
| 8 | Nucleic acid sequence encoding SEQ ID NO: 7 |
| 9 | NGO0033-ortholog of NMB1870 and having identity = 0.622 to NMB1870 |
| 10 | Nucleic acid sequence encoding SEQ ID NO: 9 |
| 11 | NMB1030 |
| 12 | Nucleic acid sequence encoding SEQ ID NO: 11 |
| 13 | NMC1183- Ortholog of NMB1030 and having identity = 0.973 to NMB1030 |
| 14 | Nucleic acid sequence encoding SEQ ID NO: 13 |
| 15 | NMA1457- Ortholog of NMB1030 and having identity = 0.973 to NMB1030 |
| 16 | Nucleic acid sequence encoding SEQ ID NO: 15 |
| 17 | NMCC_1165- Ortholog of NMB1030 and having identity = 0.963 to NMB1030 |
| 18 | Nucleic acid sequence encoding SEQ ID NO: 17 |
| 19 | NGO0558- Ortholog of NMB1030 and having identity = 0.930 to NMB1030 |
| 20 | Nucleic acid sequence encoding SEQ ID NO: 19 |
| 21 | Oant_3992- Ortholog of NMB1030 and having identity = 0.553 to NMB1030 |
| 22 | Nucleic acid sequence encoding SEQ ID NO: 21 |
| 23 | SPAB_01659- Ortholog of NMB1030 and having identity = 0.527 to NMB1030 |
| 24 | Nucleic acid sequence encoding SEQ ID NO: 23 |
| 25 | SPA1248- Ortholog of NMB1030 and having identity = 0.527 to NMB1030 |
| 26 | Nucleic acid sequence encoding SEQ ID NO: 25 |
| 27 | Aave_3505- Ortholog of NMB1030 and having identity = 0.534 to NMB1030 |
| 28 | Nucleic acid sequence encoding SEQ ID NO: 27 |
| 29 | STM1621- Ortholog of NMB1030 and having identity = 0.516 to NMB1030 |
| 30 | Nucleic acid sequence encoding SEQ ID NO: 29 |
| 31 | SC1617- Ortholog of NMB1030 and having identity = 0.516 to NMB1030 |
| 32 | Nucleic acid sequence encoding SEQ ID NO: 31 |
| 33 | Pnap_3578- Ortholog of NMB1030 and having identity = 0.518 to NMB1030 |
| 34 | Nucleic acid sequence encoding SEQ ID NO: 33 |
| 35 | t1530- Ortholog of NMB1030 and having identity = .516 to NMB1030 |
| 36 | Nucleic acid sequence encoding SEQ ID NO: 35 |
| 37 | STY1443- Ortholog of NMB1030 and having identity = .516 to NMB1030 |
| 38 | Nucleic acid sequence encoding SEQ ID NO: 37 |
| 39 | PsycPRwf_2217- Ortholog of NMB1030 and having identity = 0.542 to NMB1030 |
| 40 | Nucleic acid sequence encoding SEQ ID NO: 39 |
| 41 | NMB2091 |
| 42 | Nucleic acid sequence encoding SEQ ID NO: 41 |
| 43 | NMCC_2056- Ortholog of NMB2091 and having identity = 1.0 to NBM2091 |
| 44 | Nucleic acid sequence encoding SEQ ID NO: 43 |
| 45 | NMC2071- Ortholog of NMB2091 and having identity = 1.0 to NBM2091 |
| 46 | Nucleic acid sequence encoding SEQ ID NO: 45 |
| 47 | NMA03391- Ortholog of NMB2091 and having identity = 0.970 to NBM2091 |
| 48 | Nucleic acid sequence encoding SEQ ID NO: 47 |
| 49 | NGO1985- Ortholog of NMB2091 and having identity = 0.955 to NBM2091 |
| 50 | Nucleic acid sequence encoding SEQ ID NO: 49 |
| 51 | NMB0667 |
| 52 | Nucleic acid sequence encoding SEQ ID NO: 51 |
| 53 | NMC0615- Ortholog of NMB0667 and having identity = 1.0 to NMB0667 |
| 54 | Nucleic acid sequence encoding SEQ ID NO: 53 |
| 55 | NMCC_0620- Ortholog of NMB0667 and having identity = 0.993 to NMB0667 |
| 56 | Nucleic acid sequence encoding SEQ ID NO: 55 |
| 57 | NMA0866- Ortholog of NMB0667 and having identity = 0.986 to NMB0667 |
| 58 | Nucleic acid sequence encoding SEQ ID NO: 57 |
| 59 | NGO0236- Ortholog of NMB0667 and having identity = 0.984 to NMB0667 |
| 60 | Nucleic acid sequence encoding SEQ ID NO: 59 |
| 61 | Streptococcus agalactiae strain 98-D60C beta-antigen (bac) |
| 62 | Nucleic acid sequence encoding SEQ ID NO: 61 |
| 63 | Borrelia hermsii cspH CRASP-1 protein, isolate HS1 |
| 64 | Nucleic acid sequence encoding SEQ ID NO: 63 |
| 65 | Borrelia burgdorferi strain Sh-2-82 CRASP-2 (cspZ) protein, complete |
| 66 | Nucleic acid sequence encoding SEQ ID NO: 65 |
| 67 | Streptococcus spp. emm5 protein |
| 68 | Nucleic acid sequence encoding SEQ ID NO: 67 |
| 69 | Streptococcus pyogenes emm6 protein |
| 70 | Nucleic acid sequence encoding SEQ ID NO: 69 |
| 71 | Streptococcus pyogenes MGAS8232 emm18 protein |
| 72 | Nucleic acid sequence encoding SEQ ID NO: 71 |
| 73 | Borrelia burgdorferi 64b ErpA, protein_id = “ZP_03097639.1” |
| 74 | Nucleic acid sequence encoding SEQ ID NO: 73 |
| 75 | Borrelia burgdorferi strain BL206 plasmid cp32-2 ErpC (erpC) |
| 76 | Nucleic acid sequence encoding SEQ ID NO: 75 |
| 77 | Borrelia burgdorferi B31 erpP/BBN38 |
| 78 | Nucleic acid sequence encoding SEQ ID NO: 77 |
| 79 | Streptococcus pyogenes MGAS2096 fibronectin-binding protein |
| 80 | Nucleic acid sequence encoding SEQ ID NO: 79 |
| 81 | B. pertussis FhaD (CDS 758 . . . 1492) |
| 82 | B. pertussis FhaA (CDS 1555 . . . 4176) |
| 83 | B. pertussis FhaE (CDS 4157 . . . 5287) |
| 84 | Nucleic acid sequence encoding SEQ ID NOS: 81(CDS 758 . . . 1492), |
| | 82(CDS 1555 . . . 4176), 83 (CDS 4157 . . . 5287) |
| 85 | Borrelia hermsii isolate YOR factor H binding protein (fhbA) |
| 86 | Nucleic acid sequence encoding SEQ ID NO: 85 |
| 87 | Saccharomyces cerevisiae Gpm1p protein Tetrameric phosphoglycerate mutase, |
| | mediates the conversion of 3-phosphoglycerate to 2-phosphoglycerate during |
| | glycolysis and the reverse reaction during gluconeogenesis |
| 88 | Nucleic acid sequence encoding SEQ ID NO: 87 |
| 89 | Streptococcus pneumoniae factor H-binding inhibitor of complementsurface |
| | protein PspC (pspC11.4) |
| 90 | Nucleic acid sequence encoding SEQ ID NO: 89 |
| 91 | Leptospira interrogans serovar Pomona lenA, or LfhA, (CDS 2418 . . . 3140) |
| 92 | Nucleic acid sequence encoding SEQ ID NO: 91 (CDS 2418 . . . 3140) |
| 93 | Actinobacillus actinomycetemcomitans omp100 (CDS 602 . . . 1489) |
| 94 | Nucleic acid sequence encoding SEQ ID NO: 93 (CDS 602 . . . 1489) |
| 95 | Borrelia burgdorferi 297 plasmid cp18-2 orf28/p21 (CDS1 . . . 558) |
| 96 | Nucleic acid sequence encoding (CDS1 . . . 558) |
| 97 | Borrelia burgdorferi strain LW2 partial ospE gene for outer |
| | surface protein E strain LW2. |
| 98 | Nucleic acid sequence encoding SEQ ID NO: 97 (CDS 107 . . . >580) |
| 99 | N. meningitidis porA |
| 100 | Nucleic acid sequence encoding SEQ ID NO: 99 |
| 101 | Streptococcus pneumoniae G54 protein surface protein PspC |
| 102 | Nucleic acid sequence encoding SEQ ID NO: 101 |
| 103 | Streptococcus equiprotein Se18.9 |
| 104 | Nucleic acid sequence encoding SEQ ID NO 103 |
| 105 | Pseudomonas aeruginosa UCBPP-PA14 tufA |
| 106 | Nucleic acid sequence encoding SEQ ID NO: 105 |
| 107 | Yersinia enterocolitica YadA protein |
| 108 | Nucleic acid sequence encoding SEQ ID NO: 107 |

### MODES FOR CARRYING OUT THE INVENTION

As disclosed in WO04/032958, NMB1870, NMB1030, and NMB2091 were known to be effective antigens for vaccine compositions alone and particularly effective in combination to provide a broad range of protection. NMB 1870 was known to be a factor H binding protein, but the roles of NMB 1030 and NMB2091 in Neisseria was unknown. As set forth below, it has been determined that both NMB1030 and NMB2091 bind to factor H, just like NMB1870. Based upon this novel characterization of NMB1030 and NMB2091 as factor H binding proteins, it has been determined that factor H binding proteins work well as vaccine compositions alone, but these factor H binding proteins quite unexpectedly work well in combination to provide broad efficacy against related strains. This efficacy was demonstrated in WO04/032958, but it was not appreciated that the basis for the efficacy was the fact that these proteins were factor H binding proteins.

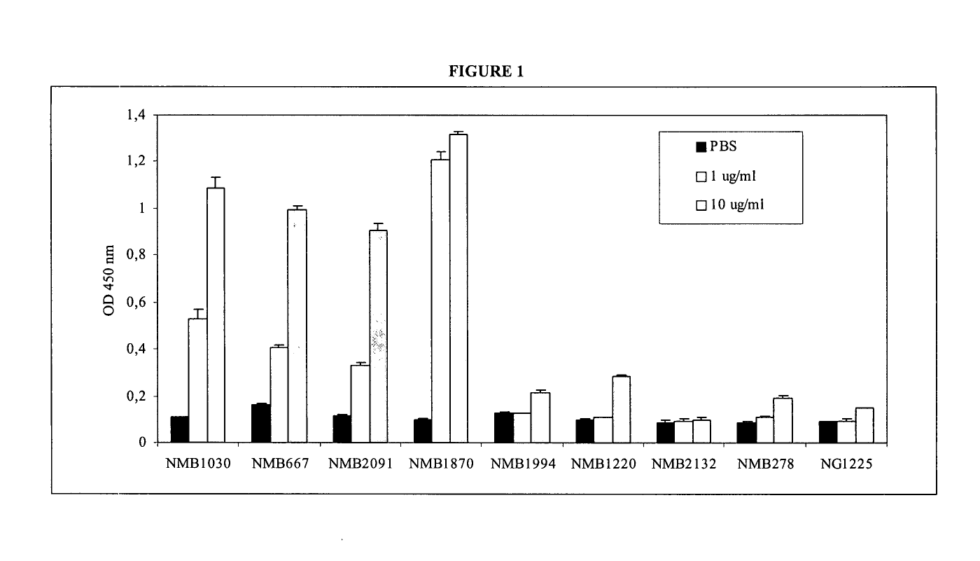

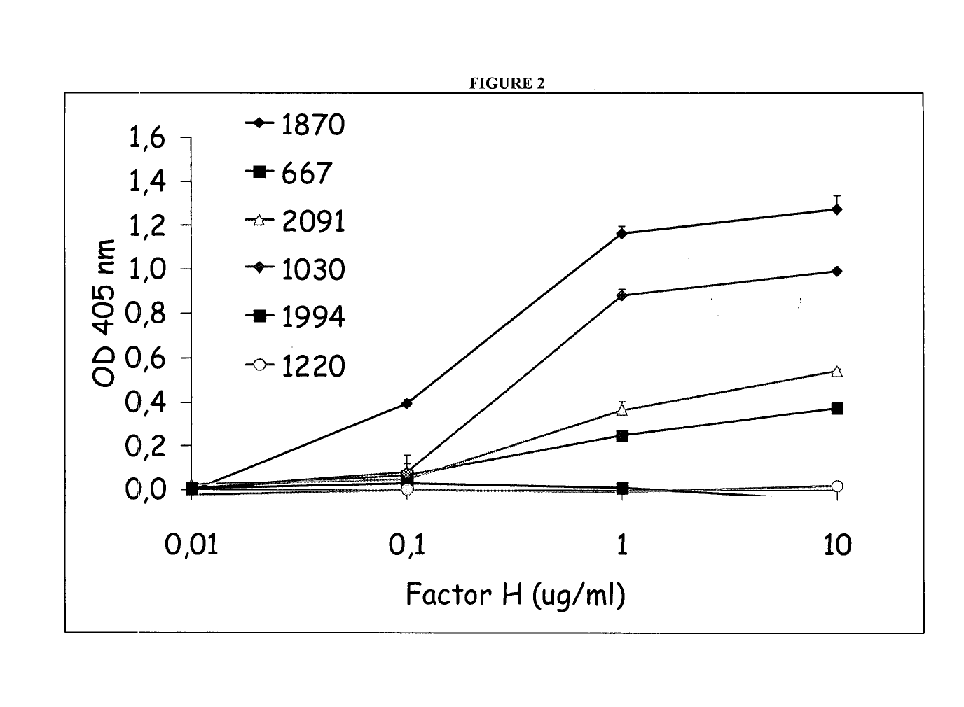

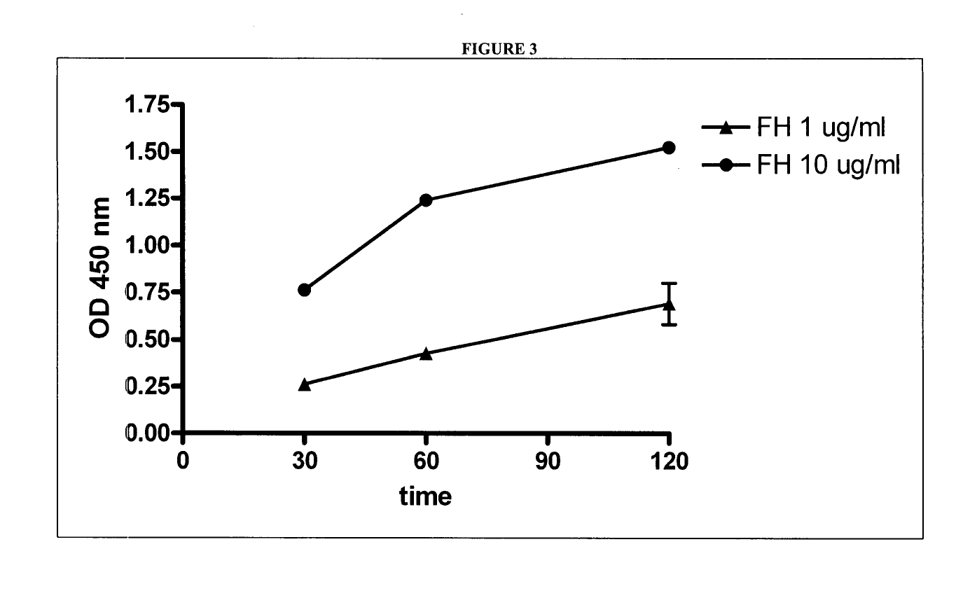

FIG. 1 shows binding assays with different N. meningitidis serogroup B antigens and one N. gonorrhoeae antigen. As expected, NMB 1870 shows a significant degree of binding to human factor H. Unexpectedly, three additional antigens also showed binding to human factor H-NMB1030, NMB0667 and NMB2091. The binding activity was confirmed and further defined through using additional concentrations of factor H to assay the dose response (FIG. 2) and through assaying the binding over time for one of the newly identified factor H binding proteins (NMB1030) (FIG. 3). FIGS. 2 and 3 confirm that NMB1030, NMB0667 and NMB2091 bind to factor H, albeit with slightly lower affinities than NMB 1870.

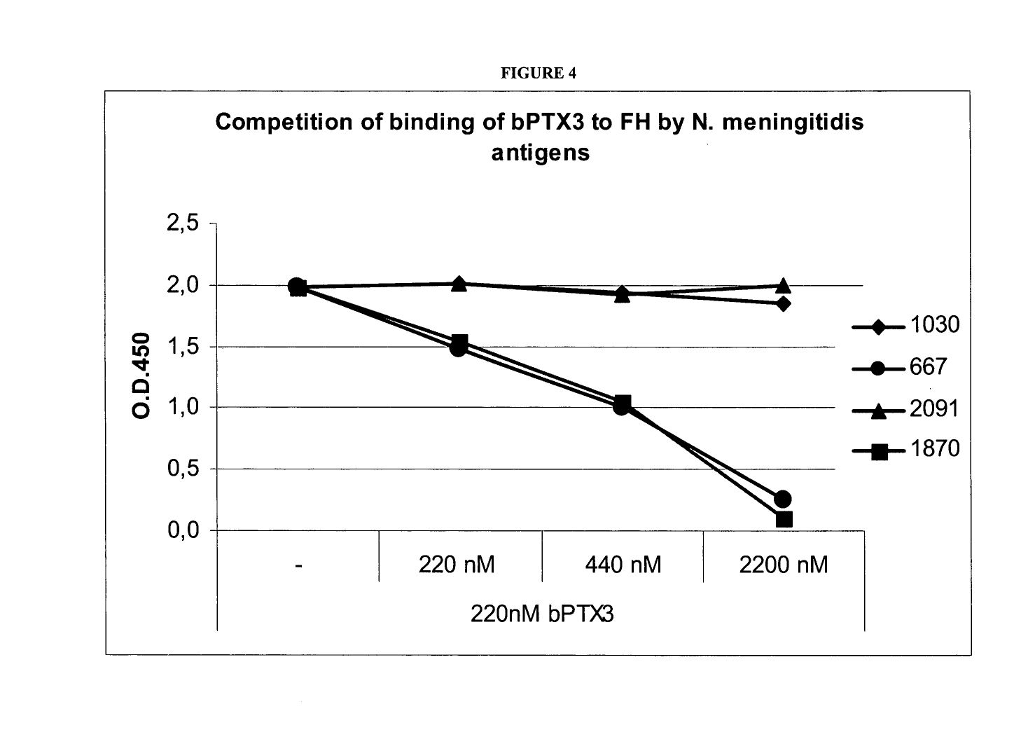

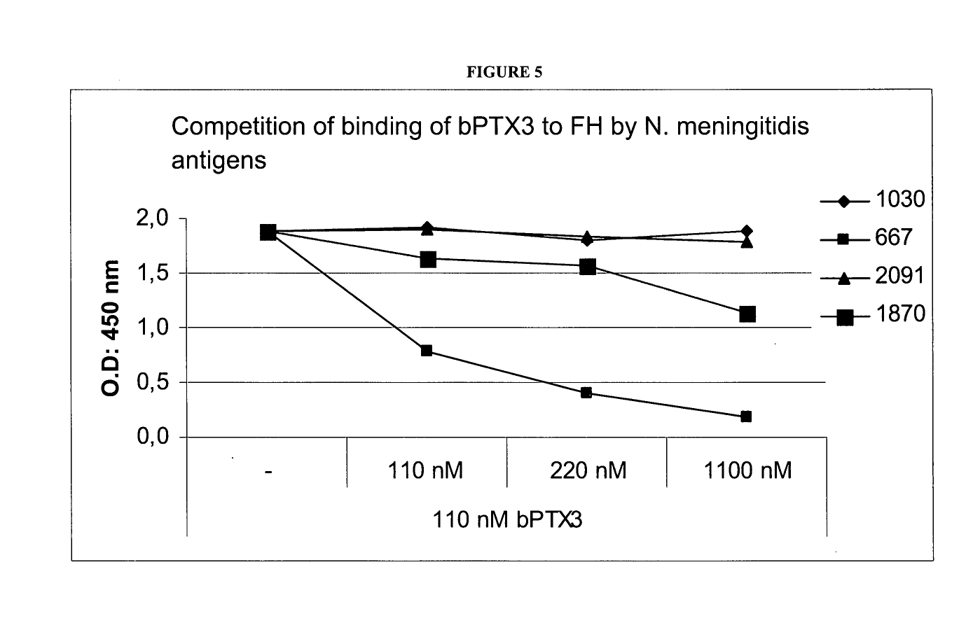

Competitive binding was assayed using the same assay to measure binding where increasing amounts of bPTX3 (one of the natural binding partners of factor H in vivo) were added. As can be seen from both FIGS. 4 and 5, increasing amounts of bPTX3 competed with the binding of both NMB 1870 and NMB0667 to factor H. This shows that NMB1870 and NMB0667 bind to the same or to overlapping sites on factor H, while NMB1030 and NMB2091 bind to different portions of factor H. This in turn shows that efficacy for use in the multiple factor H binding protein compositions of the present invention does not depend upon the factor H binding proteins binding similar sites on factor H or having the same effect upon binding of factor H.

It will be understood that the invention has been described by way of example only and modifications may be made whilst remaining within the scope and spirit of the invention.

### REFERENCES

### The Contents of which are Hereby Incorporated in Full

[1] Needleman & Wunsch (1970) J. Mol. Biol. 48, 443-453.
[2] Rice et al. (2000) Trends Genet. 16:276-277.
[3] Geysen et al. (1984) PNAS USA 81:3998-4002.
[4] Carter (1994) Methods Mol Biol 36:207-23.
[5] Jameson, B A et al. 1988, CABIOS 4(1):181-186.
[6] Raddrizzani & Hammer (2000) Brief Bioinform 1(2):179-89.
[7] Bublil et al. (2007) Proteins 68(1):294-304.
[8] De Lalla et al. (1999) J. Immunol. 163:1725-29.
[9] Kwok et al. (2001) Trends Immunol 22:583-88.
[10] Brusic et al. (1998) Bioinformatics 14(2):121-30
[11] Meister et al. (1995) Vaccine 13(6):581-91.
[12] Roberts et al. (1996) AIDS Res Hum Retroviruses 12(7):593-610.
[13] Maksyutov & Zagrebelnaya (1993) Comput Appl Biosci 9(3):291-7.
[14] Feller & de la Cruz (1991) Nature 349(6311):720-1.
[15] Hopp (1993) Peptide Research 6:183-190.
[16] Welling et al. (1985) FEBS Lett. 188:215-218.
[17] Davenport et al. (1995) Immunogenetics 42:392-297.
[18] Tsurui & Takahashi (2007) J Pharmacol Sci. 105(4):299-316.
[19] Tong et al. (2007) Brief Bioinform. 8(2):96-108.
[20] Schirle et al. (2001) J Immunol Methods. 257(1-2):1-16.
[21] Chen et al. (2007) Amino Acids 33(3):423-8.
[22] U.S. Pat. No. 5,707,829
[23] Current Protocols in Molecular Biology (F. M. Ausubel et al. eds., 1987) Supplement 30.
[24] Vaccine Design: The Subunit and Adjuvant Approach (eds. Powell & Newman) Plenum Press 1995 (ISBN 0-306-44867-X).
[25] Vaccine Adjuvants: Preparation Methods and Research Protocols (Volume 42 of Methods in Molecular Medicine series). ISBN: 1-59259-083-7. Ed. O'Hagan.
[26] U.S. Pat. No. 6,355,271.
[27] WO00/23105.
[28] WO90/14837.
[29] WO90/14837.
[30] Podda & Del Giudice (2003) Expert Rev Vaccines 2:197-203.
[31] Podda (2001) Vaccine 19: 2673-2680.
[32] Vaccine Design: The Subunit and Adjuvant Approach (eds. Powell & Newman) Plenum Press 1995 (ISBN 0-306-44867-X).
[33] Vaccine Adjuvants: Preparation Methods and Research Protocols (Volume 42 of Methods in Molecular Medicine series). ISBN: 1-59259-083-7. Ed. O'Hagan.
[34] Allison & Byars (1992) Res Immunol 143:519-25.
[35] Hariharan et al. (1995) Cancer Res 55:3486-9.
[36] US-2007/014805.
[37] Suli et al. (2004) Vaccine 22(25-26):3464-9.
[38] WO95/11700.
[39] U.S. Pat. No. 6,080,725.
[40] WO2005/097181.
[41] WO2006/113373.
[42] Han et al. (2005) Impact of Vitamin E on Immune Function and Infectious Diseases in the Aged at Nutrition, Immune functions and Health EuroConference, Paris, 9-10 Jun. 2005.
[43] U.S. Pat. No. 6,630,161.
[44] U.S. Pat. No. 5,057,540.
[45] WO96/33739.
[46] EP-A-0109942.
[47] WO96/11711.
[48] WO00/07621.
[49] Barr et al. (1998) Advanced Drug Delivery Reviews 32:247-271.
[50] Sjolanderet et al. (1998) Advanced Drug Delivery Reviews 32:321-338.
[51] Niikura et al. (2002) Virology 293:273-280.
[52] Lenz et al. (2001) J Immunol 166:5346-5355.
[53] Pinto et al. (2003) J Infect Dis 188:327-338.
[54] Gerber et al. (2001) J Virol 75:4752-4760.
[55] WO03/024480.
[56] WO03/024481.
[57] Gluck et al. (2002) Vaccine 20:B10-B16.
[58] EP-A-0689454.
[59] Johnson et al. (1999) Bioorg Med Chem Lett 9:2273-2278.
[60] Evans et al. (2003) Expert Rev Vaccines 2:219-229.
[61] Meraldi et al. (2003) Vaccine 21:2485-2491.
[62] Pajak et al. (2003) Vaccine 21:836-842.
[63] Kandimalla et al. (2003) Nucleic Acids Research 31:2393-2400.
[64] WO02/26757.
[65] WO99/62923.
[66] Krieg (2003) Nature Medicine 9:831-835.
[67] McCluskie et al. (2002) FEMS Immunology and Medical Microbiology 32:179-185.
[68] WO98/40100.
[69] U.S. Pat. No. 6,207,646.
[70] U.S. Pat. No. 6,239,116.
[71] U.S. Pat. No. 6,429,199.
[72] Kandimalla et al. (2003) Biochemical Society Transactions 31 (part 3):654-658.
[73] Blackwell et al. (2003) J Immunol 170:4061-4068.
[74] Krieg (2002) Trends Immunol 23:64-65.
[75] WO01/95935.
[76] Kandimalla et al. (2003) BBRC 306:948-953.
[77] Bhagat et al. (2003) BBRC 300:853-861.
[78] WO03/035836.
[79] WO01/22972.
[80] Schellack et al. (2006) Vaccine 24:5461-72.
[81] WO95/17211.
[82] WO98/42375.
[83] Beignon et al. (2002) Infect Immun 70:3012-3019.
[84] Pizza et al. (2001) Vaccine 19:2534-2541.
[85] Pizza et al. (2000) Int J Med Microbiol 290:455-461.
[86] Scharton-Kersten et al. (2000) Infect Immun 68:5306-5313.
[87] Ryan et al. (1999) Infect Immun 67:6270-6280.
[88] Partidos et al. (1999) Immunol Lett 67:209-216.
[89] Peppoloni et al. (2003) Expert Rev Vaccines 2:285-293.
[90] Pine et al. (2002) J Control Release 85:263-270.
[91] Tebbey et al. (2000) Vaccine 18:2723-34.
[92] Domenighini et al. (1995) Mol Microbiol 15:1165-1167.
[93] WO99/40936.
[94] WO99/44636.
[95] Singh et al] (2001) J Cont Release 70:267-276.
[96] WO99/27960.
[97] U.S. Pat. No. 6,090,406.
[98] U.S. Pat. No. 5,916,588.
[99] EP-A-0626169.
[100] WO99/52549.
[101] WO01/21207.
[102] WO01/21152.
[103] Andrianov et al. (1998) Biomaterials 19:109-115.
[104] Payne et al. (1998) Adv Drug Delivery Review 31:185-196.
[105] U.S. Pat. No. 4,680,338.
[106] U.S. Pat. No. 4,988,815.
[107] WO92/15582.
[108] Stanley (2002) Clin Exp Dermatol 27:571-577.
[109] Wu et al. (2004) Antiviral Res. 64(2):79-83.
[110] Vasilakos et al. (2000) Cell Immunol. 204(1):64-74.
[111] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[112] Jones (2003) Curr Opin Investig Drugs 4:214-218.
[113] WO03/011223.
[114] Johnson et al. (1999) Bioorg Med Chem Lett 9:2273-2278.
[115] Evans et al. (2003) Expert Rev Vaccines 2:219-229.
[116] WO2004/060308.
[117] WO2004/064759.
[118] U.S. Pat. No. 6,924,271.
[119] US2005/0070556.
[120] U.S. Pat. No. 5,658,731.
[121] U.S. Pat. No. 5,011,828.
[122] WO2004/87153.
[123] U.S. Pat. No. 6,605,617.
[124] WO02/18383.
[125] WO2004/018455.
[126] WO03/082272.
[127] Wong et al. (2003) J Clin Pharmacol 43(7):735-42.
[128] US2005/0215517.
[129] Dyakonova et al. (2004) Int Immunopharmacol 4(13):1615-23.
[130] FR-2859633.
[131] Signorelli & Hadden (2003) Int Immunopharmacol 3(8):1177-86.
[132] WO2004/064715.
[133] De Libero et al, Nature Reviews Immunology, 2005, 5: 485-496
[134] U.S. Pat. No. 5,936,076.
[135] Oki et al, J. Clin. Investig., 113: 1631-1640
[136] US2005/0192248
[137] Yang et al, Angew. Chem. Int. Ed, 2004, 43: 3818-3822
[138] WO2005/102049
[139] Goff et al, J. Am. Chem., Soc., 2004, 126: 13602-13603
[140] WO03/105769
[141] Cooper (1995) Pharm Biotechnol 6:559-80.
[142] WO99/11241.
[143] WO94/00153.
[144] WO98/57659.
[145] European patent applications 0835318, 0735898 and 0761231.
[146] Donnelly et al. (1997) Annu Rev Immunol 15:617-648.
[147] Strugnell et al. (1997) Immunol Cell Biol 75(4):364-369.
[148] Cui (2005) Adv Genet. 54:257-89.
[149] Robinson & Torres (1997) Seminars in Immunol 9:271-283.
[150] Brunham et al. (2000) J Infect Dis 181 Suppl 3:S538-43.
[151] Svanholm et al. (2000) Scand J Immunol 51(4):345-53.
[152] DNA Vaccination—Genetic Vaccination (1998) eds. Koprowski et al. (ISBN 3540633928).
[153] Gene Vaccination: Theory and Practice (1998) ed. Raz (ISBN 3540644288).
[154] Findeis et al., Trends Biotechnol. (1993) 11:202
[155] Chiou et al. (1994) Gene Therapeutics: Methods And Applications Of Direct Gene Transfer. ed. Wolff
[156] Wu et al., Biol. Chem. (1988).263:621
[157] Wu et al., J. Biol. Chem. (1994) 269:542
[158] Zenke et al., Proc. Natl. Acad. Sci. (USA) (1990) 87:3655
[159] Wu et al., J. Biol. Chem. (1991) 266:338
[160] Jolly, Cancer Gene Therapy (1994) 1:51
[161] Kimura, Human Gene Therapy (1994) 5:845
[162] Connelly, Human Gene Therapy (1995) 1:185
[163] Kaplitt, Nature Genetics (1994) 6:148
[164] WO 90/07936.
[165] WO 94/03622.
[166] WO 93/25698.
[167] WO 93/25234.
[168] U.S. Pat. No. 5,219,740.
[169] WO 93/11230.
[170] WO 93/10218.
[171] U.S. Pat. No. 4,777,127.
[172] GB Patent No. 2,200,651.
[173] EP-A-0345242.
[174] WO 91/02805.
[175] WO 94/12649.
[176] WO 93/03769.
[177] WO 93/19191.
[178] WO 94/28938.
[179] WO 95/11984.
[180] WO 95/00655.
[181] Curiel, Hum. Gene Ther. (1992) 3:147
[182] Wu, J. Biol. Chem. (1989) 264:16985
[183] U.S. Pat. No. 5,814,482.
[184] WO 95/07994.
[185] WO 96/17072.
[186] WO 95/30763.
[187] WO 97/42338.
[188] WO 90/11092.
[189] U.S. Pat. No. 5,580,859
[190] U.S. Pat. No. 5,422,120
[191] WO 95/13796.
[192] WO 94/23697.
[193] WO 91/14445.
[194] EP-0524968.
[195] Philip, Mol. Cell. Biol. (1994) 14:2411
[196] Woffendin, Proc. Natl. Acad. Sci. (1994) 91:11581
[197] U.S. Pat. No. 5,206,152.
[198] WO 92/11033.
[199] U.S. Pat. No. 5,149,655.
[200] Brandt et al. (2006) J Antimicrob Chemother. 58(6):1291-4. Epub 2006 Oct. 26
[201] Winter et al., (1991) Nature 349:293-99
[202] U.S. Pat. No. 4,816,567.
[203] Inbar et al., (1972) Proc. Natl. Acad. Sci. U.S.A. 69:2659-62.
[204] Ehrlich et al., (1980) Biochem 19:4091-96.
[205] Huston et al., (1988) Proc. Natl. Acad. Sci. U.S.A. 85:5897-83.
[206] Pack et al., (1992) Biochem 31, 1579-84.
[207] Cumber et al., (1992) J. Immunology 149B, 120-26.
[208] Riechmann et al., (1988) Nature 332, 323-27.
[209] Verhoeyan et al., (1988) Science 239, 1534-36.
[210] GB 2,276,169.
[211] Gennaro (2000) Remington: The Science and Practice of Pharmacy. 20th edition, ISBN: 0683306472.
[212] Methods In Enzymology (S. Colowick and N. Kaplan, eds., Academic Press, Inc.)
[213] Handbook of Experimental Immunology, Vols. I-IV (D. M. Weir and C. C. Blackwell, eds, 1986, Blackwell Scientific Publications)
[214] Sambrook et al. (2001) Molecular Cloning: A Laboratory Manual, 3rd edition (Cold Spring Harbor Laboratory Press).
[215] Handbook of Surface and Colloidal Chemistry (Birdi, K. S. ed., CRC Press, 1997)
[216] Ausubel et al. (eds) (2002) Short protocols in molecular biology, 5th edition (Current Protocols).
[217] Molecular Biology Techniques: An Intensive Laboratory Course, (Ream et al., eds., 1998, Academic Press)
[218] PCR (Introduction to Biotechniques Series), 2nd ed. (Newton & Graham eds., 1997, Springer Verlag)
[219] Current Protocols in Molecular Biology (F. M. Ausubel et al., eds., 1987) Supplement 30
[220] Smith & Waterman (1981) Adv. Appl. Math. 2: 482-489.

1. A composition comprising at least two factor H binding proteins with the proviso that the two factor H binding proteins are not NMB1030 and NMB2091, NMB2091 and NMB1870, or NMB 1030 and NMB 1870.

2. A composition comprising NMB0667 and a second factor H binding protein.

3. The composition of claim 1 or 2 further comprising an adjuvant.

4. The composition of claim 1 or 2 wherein the factor H binding proteins are Neisserial proteins.

5. The composition of claim 1 or 2 wherein the factor H binding proteins are Neisserial meningitidis proteins.

6. The composition of claim 1 or 2 wherein a least one factor H binding protein is selected from a polypeptide comprising an amino acid sequence that:
(a) is identical to any one of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(b) has from 1 to 10 single amino acid alterations compared to (a);
(c) has at least 85% sequence identity to any one of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(d) is a fragment of at least 10 consecutive amino acids of any of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
or
(e) when aligned with any of SEQ ID NOs: SEQ ID NOs: 1, 3, 5, 7, 9, 11, 13, 15, 17, 19; 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107 using a pairwise alignment algorithm, each moving window of x amino acids from N-terminus to C-terminus has at least x* y identical aligned amino acids, where x is 30 and y; is 0.75.

7. The composition of claim 3 wherein the factor H binding proteins are Neisserial proteins.

8. The composition of claim 3 wherein the factor H binding proteins are Neisserial meningitidis proteins.

9. The composition of claim 3 wherein a least one factor H binding protein is selected from a polypeptide comprising an amino acid sequence that:
(a) is identical to any one of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(b) has from 1 to 10 single amino acid alterations compared to (a);
(c) has at least 85% sequence identity to any one of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
(d) is a fragment of at least 10 consecutive amino acids of any of SEQ ID NOs: SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107;
or
(e) when aligned with any of SEQ ID NOs: SEQ ID NOs: 1, 3, 5, 7, 9, 11, 13, 15, 17, 19; 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81-83, 85, 87, 89, 91, 93, 95, 97, 99, 101, 103, 105, 107 using a pairwise alignment algorithm, each moving window of x amino acids from N-terminus to C-terminus has at least x* y identical aligned amino acids, where x is 30 and y; is 0.75.
